(19) United States
(12) Patent Application Publication
(10) Pub. No.: US 2025/0207110 A1
(43) Pub. Date: Jun. 26, 2025

(54) MICROBIOLOGICAL PRODUCTION OF SHORT FATTY ACIDS AND USES THEREOF

(71) Applicant: JOHANN WOLFGANG GOETHE-UNIVERSITÄT FRANKFURT AM MAIN, FrankFurt Am Main (DE)

(72) Inventors: Martin GRININGER, Frankfurt (DE); Jan GAJEWSKI, Frankfurt (DE); Eckhard BOLES, Darmstadt (DE); Renata PAVLOVIC, Zedge (SG)

(21) Appl. No.: 19/077,728

(22) Filed: Mar. 12, 2025

Related U.S. Application Data
(62) Division of application No. 17/358,163, filed on Jun. 25, 2021.
Application No. 17/358,163 is a continuation of application No. 15/561,201, filed on Sep. 25, 2017, now Pat. No. 11,078,468.

(30) Foreign Application Priority Data
Apr. 1, 2015 (EP) 15162192.7

(51) Int. Cl. C12N 9/10; C12N 15/82; C12P 7/04; C12P 7/62; C12P 7/6409; C12P 7/649

(52) U.S. Cl. CPC C12N 9/1029; C12N 15/82; C12P 7/04; C12P 7/62; C12P 7/6409; C12P 7/649; Y02E 50/10

(57) ABSTRACT

The present invention relates to proteins involved in fatty acid synthesis, such as fatty acid synthases (FAS) variants, comprising one or more polypeptide chains, wherein the polypeptide chain(s) comprise one or more subunits comprising a malonyl/palmitoyl transferase domain (MPT domain), acetyl transferase domain (AT domain), and ketoacyl synthase domain (KS domain), and at least one amino acid substitution in the MPT domain at a position corresponding to R130, in the AT domain at a position corresponding to I306, and/or in the KS domain, preferably in the acyl binding channel and/or at KS domain binding site to ACP, to modulate affinities of acyl intermediates, and optionally further amino acid substitution(s). The present invention relates to the respective polypeptide domains, nucleic acid molecules encoding the proteins (or the polypeptide domains) and host cells containing such nucleic acid molecules. The present invention relates to a method for the production of short fatty acids.

### CROSS REFERENCE TO A RELATED APPLICATION

This application is a Divisional application of co-pending U.S. patent application Ser. No. 17/358,163, filed Jun. 25, 2021, which is a Continuation application of U.S. patent application Ser. No. 15/561,201, filed Sep. 25, 2017 (now U.S. Pat. No. 11,078,468), which is a National Stage Application of International Application Number PCT/EP2016/057167, filed Mar. 31, 2016, which claims priority to European Patent Application No. 15162192.7, filed Apr. 1, 2015, all of which are incorporated herein by reference in their entirety.

### SEQUENCE LISTING

The Sequence Listing for this application is labeled “SeqList-10Mar25.xml”, which was created on Mar. 10, 2025, and is 43,292 bytes. The entire content is incorporated herein by reference in its entirety.

### FIELD OF THE INVENTION

The present invention relates to proteins involved in fatty acid synthesis, such as fatty acid synthases (FAS) variants, comprising one or more polypeptide chains, wherein said polypeptide chain(s) comprise one or more subunits comprising a malonyl/palmitoyl transferase domain (MPT domain), acetyl transferase domain (AT domain), and ketoacyl synthase domain (KS domain), and at least one amino acid substitution in the MPT domain at a position corresponding to R130, in the AT domain at a position corresponding to I306, and/or in the KS domain, preferably in the acyl binding channel and/or at KS domain binding site to ACP, to modulate affinities of acyl intermediates, and optionally further amino acid substitution(s). The present invention relates to the respective polypeptide domains.

### BACKGROUND OF THE INVENTION

The present invention further relates to nucleic acid molecules encoding the proteins (or the polypeptide domains) and to host cells containing said nucleic acid molecules. The present invention further relates to a method for the production of short fatty acids, CoA esters of short fatty acids, ethyl esters of short fatty acids, esters of short fatty acids with other metabolites, and/or enzyme bound short fatty acids (C₆ to C₁₂), comprising the expression of said nucleic acid molecules, preferably in said host cells. The present invention further relates to a method for the production of biofuels, flavoring compounds and/or fine chemicals, comprising the expression of said nucleic acid molecules, preferably in said host cells. The present invention also relates to the use of the proteins, nucleic acids molecule or host cells for the bulk production of short fatty acids (C₆ to C₁₂), the specific production of C₆ fatty acids and/or C₈ fatty acids, the bulk production of CoA esters of short fatty acids (C₆ to C₁₂), the specific production of C₆-CoA esters and/or C₈-CoA esters, the bulk production of ethyl esters of short fatty acids (C₆ to C₁₂), the specific production of C₆ fatty acid ethyl esters and/or C₈ fatty acid ethyl esters, the bulk production of esters of short fatty acids (C₆ to C₁₂) with other metabolites, the specific production of C₆ fatty acid esters with other metabolites and/or C₈ fatty acid esters with other metabolites, the bulk production of enzyme bound short fatty acids (C₆ to C₁₂), the specific production of enzyme bound C₆ fatty acids and/or enzyme bound C₈ fatty acids, the production of biofuels, fine chemicals and/or flavoring substances.

With the rising demand for industrial products from a growing world population, the call for chemicals from renewable sources has become louder. Especially the efforts have been intensified, where fatty acids (FA) can play a crucial role as a platform chemical in the production of fine chemicals or even to replace fossil derived fuel by biofuels (Runguphan & Keasling, 2014; Choi & Lee, 2013). Accordingly, the production of FA in microorganisms has been investigated extensively, constantly pushing the limits: Yields have been shown in S. cerevisiae of up to 400 mg/L free FA (Runguphan & Keasling, 2014) and more recently even 2.2 g/L (Leber et al., 2015) (in both cases products were mostly in the long chain range); and up to 4.8 g/L in E. coli (predominantly C₁₄ and C₁₆) (Liu et al., 2012).

In microorganisms themselves, FA serve several purposes, mainly as a part of membranes, in signaling but also in energy storage. Their de-novo production is tightly regulated and conducted by an enzyme group, the fatty acid synthases (FASs) (Tehlivets et al., 2007). Throughout all organisms, their reaction mechanisms and their chemistry are essentially the same: A ketoacyl synthase (KS) is responsible for the elongation of an acyl chain starter molecule, typically an acetyl-CoA, with malonyl. The resulting β-ketoacyl intermediate is then processed in a series of reaction steps, in the ketoacyl reductase (KR), the dehydratase (DH) and enoyl reductase (ER), to a fully reduced acyl chain. This acyl chain which is now extended by two carbons, serves as a starter for the next cycle. The process repeats itself until the final product is cleft off.

As far as the overall structural organization of fatty acid synthases (FASs) is concerned, two types are distinguished: In type I FASs, all necessary enzymatic functions of FA production are concentrated in one multienzymatic complex, whereas in type II FAS systems, each reaction is catalyzed by a separate enzyme. Type II FASs are found in bacteria, while type I FASs are typical for few actinobacteria and all eukaryotic organisms, among these also S. cerevisiae.

In detail, in the S. cerevisiae FAS, one set of domains is distributed on two genes, fas1 and fas2. Multiple copies of the corresponding two polypeptide chains form the heterododecameric α₆β₆ 2.6 MDa complex, which has been object of extensive x-ray structural analysis with resolutions up to 3.1 Å (see e.g., Jenni et al., 2007; Johansson et al., 2008). Its interpretation has led to substantially new insights in the reaction mechanisms of the whole FAS enzyme family (Grininger 2014; Beld et al., 2015). Above that, the kinetic parameters of S. cerevisiae FAS have been studied for decades.

The product distribution of the S. cerevisiae FAS is naturally in the long chain range of C₁₆ and C₁₈ (Tehlivets et al., 2007) and not directly suitable for applications where short FA are needed, as for instance biofuels in the petrol range. For this purpose, products typically have to have a length of C₄ to C₁₂ (Peralta-Yahya et al., 2012). Previous engineering efforts for the production of short FA have heavily relied on the utilization of thioesterases (TEs) with known specificities for short chain products (Beld et al., 2015), e.g. in a proof of principle study for the alkane production in E. coli (Choi & Lee, 2013) or the production of the precursors, short FA, in S. cerevisiae (with total yields up to 111 mg/L) (Leber & da Silva, 2014). In contrast, the rational engineering for the production of short FA was believed to be hard to achieve (Beld et al., 2015; Leber & da Silva, 2014).

US 2003/0145350 A1 discloses DNA sequences which code for a protein having the enzymatic activity of a beta-ketoacyl ACP synthase (KAS) of the enzyme complex of the fatty acid synthase (FAS). US 2003/0145350 A1 further discloses transgenic plants and micro-organisms which containing said nucleic acid sequences and a method for influencing the fatty acid pattern and/or for increasing the fatty acid content, especially the content of short and middle chain fatty acids, in plants, especially in seed tissues that synthesize and/or store triacylglycerols, as well as in micro-organisms, especially bacteria and algae. The method comprises the expression of proteins having the activity of a KAS of the enzyme complex or the fatty acid synthase in transgenic plants or micro-organisms.

There is a need in the art for further as well as improved means and methods for producing fatty acids in microorganisms, in particular fatty acids that are suitable for biofuel production, fine chemicals and other compounds.

### SUMMARY OF THE INVENTION

According to the present invention this object is solved by a polypeptide or protein involved in fatty acid synthesis, said polypeptide or protein comprising one or more polypeptide chains, wherein said polypeptide chain(s) comprise
(i) one or more subunits comprising the amino acid sequences of
   SEQ ID NO: 1 (malonyl/palmitoyl transferase domain, MPT domain);
   SEQ ID NO: 2 (acetyl transferase domain, AT domain), and/or
   SEQ ID NO: 3 (ketoacyl synthase domain, KS domain);
(ii) at least one amino acid substitution
   in the MPT domain at a position corresponding to R130 of the amino acid sequence of SEQ ID NO: 1;
   in the AT domain at a position corresponding to I306 of the amino acid sequence of SEQ ID NO: 2;
   and/or
   in the KS domain, preferably in the acyl binding channel, preferably selected from a position corresponding to G236, M237 and F265 of the amino acid sequence of SEQ ID NO: 3;
wherein the amino acid sequence comprising the at least one amino acid substitution has at least 60%, or preferably at least 70% or 80% or 90% or 95% sequence identity to the respective amino acid sequence of SEQ ID NO: 1, 2 and/or 3.

According to the present invention this object is solved by a polypeptide or protein involved in fatty acid synthesis, as defined above, comprising at least one further amino acid substitution in the KS domain, preferably selected from a position corresponding to Q193, N258 and D259 of the amino acid sequence of SEQ ID NO: 3.

According to the present invention this object is solved by a polypeptide domain comprising
(i) one or more subunits comprising the amino acid sequences of
   SEQ ID NO: 1 (malonyl/palmitoyl transferase domain, MPT domain);
   SEQ ID NO: 2 (acetyl transferase domain, AT domain), or
   SEQ ID NO: 3 (ketoacyl synthase domain, KS domain);
(ii) at least one amino acid substitution
   in the MPT domain at a position corresponding to R130 of the amino acid sequence of SEQ ID NO: 1;
   in the AT domain at a position corresponding to I306 of the amino acid sequence of SEQ ID NO: 2;
   and/or
   in the KS domain, preferably in the acyl binding channel, preferably selected from a position corresponding to G236, M237 and F265 of the amino acid sequence of SEQ ID NO: 3;
wherein the amino acid sequence comprising the at least one amino acid substitution has at least 60%, or preferably at least 70% or 80% or 90% or 95% sequence identity to the respective amino acid sequence of SEQ ID NO: 1, 2 and/or 3.

According to the present invention this object is solved by a polypeptide domain, as defined above, comprising at least one further amino acid substitution in the KS domain, preferably selected from a position corresponding to Q193, N258 and D259 of the amino acid sequence of SEQ ID NO: 3.

According to the present invention this object is solved by a nucleic acid molecule coding for a protein of the present invention or a polypeptide domain of the present invention.

According to the present invention this object is solved by a host cell, containing a nucleic acid molecule of the present invention and preferably expressing said nucleic acid molecule, wherein said host cell is preferably selected from a bacterial cell or a fungus cell, more preferably a yeast cell, or an algae cell.

According to the present invention this object is solved by a method for the production of short fatty acids, CoA esters of short fatty acids, ethyl esters of short fatty acids, esters of short fatty acids with other metabolites, and/or enzyme bound short fatty acids (C₆ to C₁₂), comprising the expression of a nucleic acid molecule according to the present invention, preferably in a host cell according to the present invention.

According to the present invention this object is solved by a method for the production of biofuels, flavoring compounds and/or fine chemicals comprising the expression of a nucleic acid molecule according to the present invention, preferably in a host cell according to the present invention.

According to the present invention this object is solved by using a protein or polypeptide according to the present invention, a nucleic acid molecule according to the present invention, or a host cell according to the present invention for the bulk production of short fatty acids (C₆ to C₁₂).

According to the present invention this object is solved by using a protein or polypeptide according to the present invention, a nucleic acid molecule according to the present invention, or a host cell according to the present invention for the specific production of C₆ fatty acids.

According to the present invention this object is solved by using a protein or polypeptide according to the present invention, a nucleic acid molecule according to the present invention, or a host cell according to the present invention for the specific production of C₈ fatty acids.

According to the present invention this object is solved by using a protein or polypeptide according to the present invention, a nucleic acid molecule according to the present invention, or a host cell according to the present invention for the bulk production of CoA esters of short fatty acids (C₆ to C₁₂).

According to the present invention this object is solved by using a protein or polypeptide according to the present invention, a nucleic acid molecule according to the present invention, or a host cell according to the present invention for the specific production of C₆-CoA esters.

According to the present invention this object is solved by using a protein or polypeptide according to the present invention, a nucleic acid molecule according to the present invention, or a host cell according to the present invention for the specific production of C₈-CoA esters.

According to the present invention this object is solved by using a protein or polypeptide according to the present invention, a nucleic acid molecule according to the present invention, or a host cell according to the present invention for the bulk production of ethyl esters of short fatty acids (C₆ to C₁₂) or esters of short fatty acids (C₆ to C₁₂) with other metabolites.

According to the present invention this object is solved by using a protein or polypeptide according to the present invention, a nucleic acid molecule according to the present invention, or a host cell according to the present invention for the specific production of C₆ fatty acid ethyl esters or C₆ fatty acid esters with other metabolites.

According to the present invention this object is solved by using a protein or polypeptide according to the present invention, a nucleic acid molecule according to the present invention, or a host cell according to the present invention for the specific production of C₈ fatty acid ethyl esters or C₈ fatty acid esters with other metabolites.

According to the present invention this object is solved by using a protein or polypeptide according to the present invention, a nucleic acid molecule according to the present invention, or a host cell according to the present invention for the bulk production of enzyme bound short fatty acids (C₆ to C₁₂).

According to the present invention this object is solved by using a protein or polypeptide according to the present invention, a nucleic acid molecule according to the present invention, or a host cell according to the present invention for the specific production of enzyme bound C₆ fatty acids.

According to the present invention this object is solved by using a protein or polypeptide according to the present invention, a nucleic acid molecule according to the present invention, or a host cell according to the present invention for the specific production of enzyme bound C₈ fatty acids.

According to the present invention this object is solved by using a protein or polypeptide according to the present invention, a nucleic acid molecule according to the present invention, or a host cell according to the present invention for the production of biofuels, such as short alkanes, short alkenes, short alkynes, short esters and/or alcohols.

According to the present invention this object is solved by using a protein or polypeptide according to the present invention, a nucleic acid molecule according to the present invention, or a host cell according to the present invention for the production of fine chemicals, such as natural compounds where preferably short fatty acids (C₆ to C₁₂) or their derivatives (such as CoA esters, methyl/ethyl esters, esters with other metabolites, enzyme bound fatty acids, alcohols) are used as building block(s).

According to the present invention this object is solved by using a protein or polypeptide according to the present invention, a nucleic acid molecule according to the present invention, or a host cell according to the present invention for the production of flavoring substances, such as esters from short fatty acids (C₆ to C₁₂).

### DESCRIPTION OF THE PREFERRED EMBODIMENTS OF THE INVENTION

Before the present invention is described in more detail below, it is to be understood that this invention is not limited to the particular methodology, protocols and reagents described herein as these may vary.

It is also to be understood that the terminology used herein is for the purpose of describing particular embodiments only and is not intended to limit the scope of the present invention which will be limited only by the appended claims. Unless defined otherwise, all technical and scientific terms used herein have the same meanings as commonly understood by one of ordinary skill in the art. For the purpose of the present invention, all references cited herein are incorporated by reference in their entireties.

### Fatty Acid Synthase Variants and Domains

As discussed above, the present invention provides fatty acid synthase (FAS) variants, in particular type I fatty acid synthase (type I FAS) variants.

The polypeptides or proteins of the present invention comprise one or more polypeptide chains. Said one or more polypeptide chains comprise one or more subunits comprising a malonyl/palmitoyl transferase domain (MPT domain), an acetyl transferase domain (AT domain), and/or a ketoacyl synthase domain (KS domain).

The invention provides a protein or polypeptide involved in fatty acid synthesis, preferably having fatty acid synthase activity.

The invention also provides polypeptide domain(s) which are involved in fatty acid synthesis activity, such as subunits comprising a malonyl/palmitoyl transferase domain (MPT domain), an acetyl transferase domain (AT domain), and/or a ketoacyl synthase domain (KS domain).

Fatty acid synthases or polypeptides/proteins involved in fatty acid synthesis comprise one or more polypeptide chains, such as two, three, four or more polypeptide chains.

Fatty acid synthases or polypeptides/proteins involved in fatty acid synthesis furthermore comprise several different catalytic domains or subunits. Said catalytic domains or subunits can be located on said different/one or more polypeptide chains. The catalytic domains or subunits can also be split in parts and the different parts can be located on said different/one or more polypeptide chains (such as in case for the MPT domain of the type I FAS from S. cerevisiae).

According to the invention, the polypeptides or proteins of the present invention further comprise at least one amino acid substitution in the MPT domain at a position corresponding to R130, in the AT domain at a position corresponding to I306, and/or in the KS domain, preferably in the acyl binding channel, to modulate affinities of acyl intermediates, and optionally further amino acid substitution(s).

In particular, the present invention provides a polypeptide or protein involved in fatty acid synthesis, said polypeptide comprising one or more polypeptide chains, said polypeptide chain(s) comprising
(i) at least one subunit comprising the amino acid sequences of
   SEQ ID NO: 1 (malonyl/palmitoyl transferase domain, MPT domain);
   SEQ ID NO: 2 (acetyl transferase domain, AT domain), and/or
   SEQ ID NO: 3 (ketoacyl synthase domain, KS domain);
(ii) at least one amino acid substitution
   in the MPT domain at a position corresponding to R130 of the amino acid sequence of SEQ ID NO: 1;
   in the AT domain at a position corresponding to I306 of the amino acid sequence of SEQ ID NO: 2;
   and/or
   in the KS domain, preferably in the acyl binding channel, preferably selected from a position corresponding to G236, M237 and F265 of the amino acid sequence of SEQ ID NO: 3.

In particular, the present invention provides a polypeptide or protein involved in fatty acid synthesis, as defined above, comprising at least one further amino acid substitution in the KS domain, preferably in the acyl binding channel and/or at KS domain binding site to ACP, preferably selected from a position corresponding to Q193, N258 and D259 of the amino acid sequence of SEQ ID NO: 3.

According to the present invention, the amino acid sequence comprising the at least one amino acid substitution has at least 60%, or preferably at least 70% or 80% or 90% or 95% sequence identity to the respective amino acid sequence of SEQ ID NO: 1, 2 and/or 3.

In a preferred embodiment, the amino acid sequence comprising the at least one amino acid substitution has at least 80%, or preferably at least 90% or 95% sequence identity to the respective amino acid sequence of SEQ ID NO: 3.

In particular, the present invention provides a polypeptide domain comprising
(i) one or more subunits comprising the amino acid sequences of
   SEQ ID NO: 1 (malonyl/palmitoyl transferase domain, MPT domain);
   SEQ ID NO: 2 (acetyl transferase domain, AT domain), or
   SEQ ID NO: 3 (ketoacyl synthase domain, KS domain);
(ii) at least one amino acid substitution
   in the MPT domain at a position corresponding to R130 of the amino acid sequence of SEQ ID NO: 1;
   in the AT domain at a position corresponding to I306 of the amino acid sequence of SEQ ID NO: 2;
   and/or
   in the KS domain, preferably in the acyl binding channel, preferably selected from a position corresponding to G236, M237 and F265 of the amino acid sequence of SEQ ID NO: 3;

In particular, the present invention provides a polypeptide domain, as defined above, comprising at least one further amino acid substitution in the KS domain, preferably at KS domain binding site to ACP, preferably selected from a position corresponding to Q193, N258 and D259 of the amino acid sequence of SEQ ID NO: 3.

According to the present invention, the amino acid sequence comprising the at least one amino acid substitution has at least 60%, or preferably at least 70% or 80% or 90% or 95% sequence identity to the respective amino acid sequence of SEQ ID NO: 1, 2 and/or 3.

In a preferred embodiment, the amino acid sequence comprising the at least one amino acid substitution has at least 80%, or preferably at least 90% or 95% sequence identity to the respective amino acid sequence of SEQ ID NO: 3.

In a preferred embodiment, the MPT and/or AT domain of the polypeptide(s) or protein(s) (or the polypeptide domain(s)) have an in vitro and/or in vivo transferase activity; and/or the KS domain of the polypeptide(s) or protein(s) has an in vitro and/or in vivo ketoacyl synthase activity.

This means that even though there is at least one amino acid substitution in the MPT domain, AT domain and/or KS domain, as disclosed herein, the MPT and/or AT domain of the polypeptide(s) or protein(s) have an in vitro and/or in vivo transferase activity; and/or the KS domain of the polypeptide(s) or protein(s) has an in vitro and/or in vivo ketoacyl synthase activity.

There are two principal classes of fatty acid synthases.

Type I systems utilize a single large, multifunctional enzyme and are common to both mammals and fungi (although the structural arrangement of fungal and mammalian synthases differ). A Type I fatty acid synthase system is also found in the CMN group of bacteria (corynebacteria, mycobacteria, and nocardia). In these bacteria, the FAS I system produces palmititic acid, and cooperates with the FAS II system to produce a greater diversity of lipid products. Type II is found in archaea and bacteria, and is characterized by the use of discrete monofunctional enzymes for fatty acid synthesis.

For example, mammalian FAS usually consists of a homodimer of two identical protein subunits, in which three catalytic domains in the N-terminal section (-ketoacyl synthase (KS), malonyl/acetyltransferase (MAT), and dehydratase (DH)), are separated by a core region of 600 residues from four C-terminal domains (enoyl reductase (ER), -ketoacyl reductase (KR), acyl carrier protein (ACP) and thioesterase (TE)).

In one embodiment, the protein or polypeptide of the present invention is a type I FAS, such as type I FAS of Saccharomyces cerevisiae. In the case of Saccharomyces cerevisiae, the FAS is split into two polypeptide chains, but still is considered a type I FAS since the catalytic centers are organized in one enzyme complex.

In one embodiment, the protein or polypeptide of the present invention is the type I FAS of Saccharomyces cerevisiae.

The particular type I FAS from S. cerevisiae comprises two polypeptide chains which among other domains, contain
on a first polypeptide chain FAS 1:
the malonyl/palmitoyl transferase domain, MPT domain
and
the acetyl transferase domain, AT domain
on a second polypeptide chain, FAS 2 ketoacyl synthase domain, KS domain.

In S. cerevisiae, the chains are encoded by fas1 (to be found in Genbank: M30162.1) and fas2 (to be found in Genbank: J03936.1).

SEQ ID NO: 1 shows the amino acid sequence of the MPT domain (part of sequence of Uniprot Identifier: P07149 and of P19097)

Malonyl/palmitoyl transferase (MPT domain), (sequence is split in S. cerevisiae with the splitting site indicated by *: from residue 1-347 shown here, it is part of S. cerevisiae FAS 1 (beta chain), Uniprot Identifier: P07149; from residue 348-429 shown here, it is part of S. cerevisiae FAS 2 (beta alpha), Uniprot Identifier: P19097)

(SEQ ID NO: 1)
SILDIVINNPVNLTIHFGGEKGKRIRENYSAMIFETIVDGKLKTEKIFK
EINEHSTSYTFRSEKGLLSATQFTQPALTLMEKAAFEDLKSKGLIPADA
TFAGHSLGEYAALASLADVMSIESLVEVVFYRGMTMQVAVPRDELGRSN
YGMIAINPGRVAASFSQEALQYVVERVGKRTGWLVEIVNYNVENQQYVA
AGDLRALDTVTNVLNFIKLQKIDIIELQKSLSLEEVEGHLFEIIDEASK
KSAVKPRPLKLERGFACIPLVGISVPFHSTYLMNGVKPFKSFLKKNIIK
ENVKVARLAGKYIPNLTAKPFQVTKEYFQDVYDLTGSEPIKEIIDNWEK
YEQS*MKPEVEQELAHILLTELLAYQFASPVRWIETQDVFLKDFNTERV
VEIGPSPTLAGMAQRTLKNKYESYDAALSLHREILCYS

SEQ ID NO: 2 shows the amino acid sequence of the AT domain (part of sequence of Uniprot Identifier: P07149)

Acetyl transferase (AT domain), (part of S. cerevisiae FAS 1 (beta chain), Uniprot Identifier: P07149

MDAYSTRPLTLSHGSLEHVLLVPTASFFIASQLQEQFNKILPEPTEGFA
ADDEPTTPAELVGKFLGYVSSLVEPSKVGQFDQVLNLCLTEFENCYLEG
NDIHALAAKLLQENDTTLVKTKELIKNYITARIMAKRPFDKKSNSALFR
AVGEGNAQLVAIFGGQGNTDDYFEELRDLYQTYHVLVGDLIKFSAETLS
ELIRTTLDAEKVFTQGLNILEWLENPSNTPDKDYLLSIPISCPLIGVIQ
LAHYVVTAKLLGFTPGELRSYLKGATGHSQGLVTAVAIAETDSWESFFV
SVRKAITVLFFIGVRCYEAYPNTSLPPSILEDSLENNEGVPSPMLSISN
LTQEQVQDYVNKTNSHLPAGKQVEISLVNGAKNLVVSGPPQSLYGLNLT
LRKAKAPSGLDQSRIPFSERKLKFSNRFLPVASPFHSHLLVPASDLINK
DLVKNNVSFNAKDIQIPVYDTFDGSDLRVLSGSISERIVDCIIRLPVKW
ETTTQFKATHILDFGPGGASGLGVLTHRNKDGTGVRVIVAGTLDINPDD
DYGFKQEIFDVT

SEQ ID NO: 3 shows the amino acid sequence of the KS domain (part of sequence of Uniprot Identifier: P19097)

Ketoacyl synthase (KS domain), (part of S. cerevisiae FAS 2 (alpha chain), Uniprot Identifier: P19097)

LERVIVVTGFAEVGPWGSARTRWEMEAFGEFSLEGCVEMAWIMGFISYH
NGNLKGRPYTGWVDSKTKEPVDDKDVKAKYETSILEHSGIRLIEPELFN
GYNPEKKEMIQEVIVEEDLEPFEASKETAEQFKHQHGDKVDIFEIPETG
EYSVKLLKGATLYIPKALRFDRLVAGQIPTGWNAKTYGISDDIISQVDP
ITLFVLVSVVEAFIASGITDPYEMYKYVHVSEVGNCSGSGMGGVSALRG
MFKDRFKDEPVQNDILQESFINTMSAWVNMLLISSSGPIKTPVGACATS
VESVDIGVETILSGKARICIVGGYDDFQEEGSFEFGNMKATSNTLEEFE
HGRTPAEMSRPATTTRNGFMEAQGAGIQIIMQADLALKMGVPIYGIVAM
AATATDKIGRSVPAPGKGILTTAREHHSSVKYASPNLNMKYRKRQLVTR
EAQIKDWVENELEALKLEAEEIPSEDQNEFLLERTREIHNEAESQLRAA
QQQWGNDFYKRDPRIAPLRGALATYGLTIDDLGVASFHGTSTKANDKNE
SATINEMMKHLGRSEGNPVIGVFQKFLTGHPKGAAGAWMMNGALQILNS
GIIPGNRNADNVDKILEQFEYVLYPSKTLKTDGVRAVSITSFGFGQKGG
QAIVVHPDYLYGA

As far as the overall structural organization of fatty acid synthases (FASs) is concerned, two types are distinguished: In type I FASs, all necessary enzymatic functions of fatty acid (FA) production are concentrated in one multienzymatic complex, whereas in type II FAS systems, each reaction is catalyzed by a separate enzyme. For type I FAS systems, the intermediates are always covalently bound to the multienzymatic complex leading to extremely high efficiency. Type II FASs are found in bacteria, while type I FASs are typical for few actinobacteria and all eukaryotic organisms, among these also S. cerevisiae.

In detail, in the S. cerevisiae FAS, one set of domains is distributed on two genes, fas1 (encoding the p chain or FAS1) and fas2 (encoding the a chain or FAS2). On the p chain, the AT domain, the ER domain, the DH domain and part of the MPT domain are located in the order as written here. The rest of the MPT domain, the ACP domain, the KR domain, the KS domain and the PPT domain can be found on the a chain correspondingly. Multiple copies of the corresponding two polypeptide chains form the heterododecameric α₆β₆ 2.6 MDa complex, which has been object of extensive x-ray structural analysis with resolutions up to 3.1 Å (see e.g. Jenni et al., 2007; Johansson et al., 2008). Its interpretation has led to substantially new insights in the reaction mechanisms of the whole FAS enzyme family (Grininger 2014; Beld et al., 2015). Above that, the kinetic parameters of S. cerevisiae FAS have been studied for decades.

As used herein, the term “at a position corresponding to” means the respective position in SEQ ID No: 1, 2 or 3 which, however, in related polypeptide chains can have another relative position number. The equivalent substitution can be determined by comparing a position in both sequences, which may be aligned for the purpose of comparison. The relative position of the amino acid can vary due to different length of the related polypeptide, or deletions or additions of amino acids in the related polypeptide.

The polypeptides of the present invention, in particular the type I FAS variants, have an in vitro and/or in vivo fatty acid synthase I (FAS I) enzymatic activity.

As used herein, the term “percent (%) identical” refers to sequence identity between two amino acid sequences. Identity can be determined by comparing a position in both sequences, which may be aligned for the purpose of comparison. When an equivalent position in the compared sequences is occupied by the same amino acid, the molecules are considered to be identical at that position.

As used herein, the term “functional equivalent” refers to amino acid sequences that are not 100% identical to the amino acid sequence of SEQ ID NO. 1, 2 or 3 and comprise amino acid additions and/or insertions and/or deletions and/or substitutions and/or exchanges, which do not alter or change the activity or function of the protein as compared to the protein having the amino acid sequence of SEQ ID NO: 1, 2 or 3, i.e. an “functional equivalent”, for example, encompasses an amino acid sequence with conservative amino acid substitutions or smaller deletions and/or insertions as long as these modifications do not substantially affect the in vitro and/or in vivo fatty acid synthase (FAS) enzymatic activity.

Generally, a person skilled in the art is aware of the fact that some amino acid exchanges in the amino acid sequence of a protein do not have an influence on the (secondary or tertiary) structure, function and/or activity of that protein. Amino acid sequences with such “neutral” amino acid exchanges as compared to the amino acid sequences disclosed herein fall within the scope of the present invention.

In some embodiments, the polypeptide(s) or protein(s) have one, two, three, four, five, six or more amino acid substitutions in the MPT domain, AT domain and/or KS domain.

For example, a polypeptide of the present inventions has one amino acid substitution in each of the MPT domain, AT domain and/or KS domain.

For example, a polypeptide of the present inventions has two amino acid substitutions, such as:
one amino acid substitution in each of the MPT domain and the AT domain,
one amino acid substitution in each of the MPT domain and the KS domain,
one amino acid substitution in each of the AT domain and the KS domain,
two amino acid substitutions in the MPT domain,
two amino acid substitutions in the AT domain,
two amino acid substitutions in the KS domain.

For example, a polypeptide of the present inventions has three amino acid substitutions, such as:
three amino acid substitutions in the MPT domain,
three amino acid substitutions in the AT domain,
three amino acid substitutions in the KS domain.
two amino acid substitutions in the MPT domain and one amino acid substitution in either one of the AT domain and the KS domain,
two amino acid substitutions in the AT domain and one amino acid substitution in either one of the MPT domain and the KS domain,
two amino acid substitutions in the KS domain and one amino acid substitution in either one of MPT domain and the AT domain,
one amino acid substitution in each of the MPT domain, AT domain and KS domain.

Preferably, the polypeptides or proteins according to the present invention comprise
the amino acid substitution R130K in the MPT domain (SEQ ID NO. 1), and/or
the amino acid substitution I306A in the AT domain (SEQ ID NO: 2).

Preferably, the polypeptides or proteins according to the present invention comprise amino acid substitution(s) in the acyl binding channel of the KS domain, more preferably amino acid substitution(s) G236S, M237W and/or F265Y in the KS domain (SEQ ID NO: 3).

Preferably, the polypeptides or proteins according to the present invention comprise
the amino acid substitution R130K in the MPT domain (SEQ ID NO. 1), and/or
the amino acid substitution I306A in the AT domain (SEQ ID NO: 2), and/or
the amino acid substitution(s) G236S, M237W and/or F265Y in the KS domain (SEQ ID NO: 3) and/or
the amino acid substitution(s) Q193A, Q193E, N258A, N258D and/or D259A in the KS domain (SEQ ID NO: 3).

### Disclaimer

The present invention does not encompass polypeptide domain(s) that only comprise the amino acid substitution G236S in the KS domain.

In an embodiment, where the polypeptide domain of the present invention comprises the KS domain and an amino acid substitution G236S in said KS domain (SEQ ID NO. 3), it comprises at least one additional amino acid substitution in said KS domain.

The present invention does not encompass polypeptides or proteins that only comprise the amino acid substitution G236S in the KS domain.

In an embodiment, where the polypeptide or protein of the present invention comprises the amino acid substitution G236S in the KS domain (SEQ ID NO. 3), it comprises at least one additional amino acid substitution.

Preferably, the proteins or polypeptides according to the present invention are selected from the group of
a protein comprising the amino acid substitutions I306A and G236S;
a protein comprising the amino acid substitutions I306A, R130K and F265Y;
a protein comprising the amino acid substitutions I306A, R130K and G236S;
a protein comprising the amino acid substitution R130K;
a protein comprising the amino acid substitutions I306A, R130K, G236S and M237W;
a protein comprising the amino acid substitutions I306A, G236S and M237W;
a protein comprising the amino acid substitutions G236S and M237W;
a protein comprising the amino acid substitutions I306A and R130K;
a protein comprising the amino acid substitutions R130K and G236S;
a protein comprising the amino acid substitutions I306A and F265Y;
a protein comprising the amino acid substitutions I306A, G236S and F265Y;
a protein comprising the amino acid substitutions R130K, G236S and M237W;
a protein comprising the amino acid substitutions G236S and F265Y;
a protein comprising the amino acid substitutions R130K, G236S and F265Y;
a protein comprising the amino acid substitutions I306A, R130K, G236S and F265Y;
a protein comprising the amino acid substitutions I306A, G236S, M237W and F265Y;
a protein comprising the amino acid substitution I306A;
a protein comprising the amino acid substitution M237W;
a protein comprising the amino acid substitution F265Y;
a protein comprising the amino acid substitutions I306A and M237W;
a protein comprising the amino acid substitutions R130K and M237W;
a protein comprising the amino acid substitutions R130K and F265Y;
a protein comprising the amino acid substitutions I306A, R130K and M237W;
a protein comprising the amino acid substitutions I306A, M237W and F265Y;
a protein comprising the amino acid substitutions R130K, M237W and F265Y;
a protein comprising the amino acid substitutions G236S, M237W and F265Y;
a protein comprising the amino acid substitutions M237W and F265Y;
a protein comprising the amino acid substitutions I306A, R130K, M237W and F265Y;
a protein comprising the amino acid substitutions R130K, G236S, M237W and F265Y;
a protein comprising the amino acid substitutions I306A, R130K, G236S, M237W and F265Y;
a protein comprising the amino acid substitutions I306A, R130K, G236S and D259A;
a protein comprising the amino acid substitutions I306A, R130K, G236S, M237W and D259A;
a protein comprising the amino acid substitutions I306A, R130K, G236S and N258A;
a protein comprising the amino acid substitutions I306A, R130K, G236S, M237W and N258A;
a protein comprising the amino acid substitutions I306A, R130K, G236S and N258D;
a protein comprising the amino acid substitutions I306A, R130K, G236S, M237W and N258D;
a protein comprising the amino acid substitutions I306A, R130K, G236S and Q193A;
a protein comprising the amino acid substitutions I306A, R130K, G236S, M237W and Q193A;
a protein comprising the amino acid substitutions I306A, R130K, G236S and Q193E;
a protein comprising the amino acid substitutions I306A, R130K, G236S, M237W and Q193E.

The present invention preferably provides the following proteins or polypeptides/type I FAS variants:
variant I306A/G236S;
variant I306A/R130K/F265Y;
variant I306A/R130K/G236S;
variant R130K;
variant I306A/R130K/G236S/M237W;
variant I306A/G236S/M237W;
variant G236S/M237W;
variant I306A/R130K;
variant R130K/G236S;
variant I306A/F265Y;
variant I306A/G236S/F265Y;
variant R130K/G236S/M237W;
variant G236S/F265Y;
variant R130K/G236S/F265Y;
variant I306A/R130K/G236S/F265Y;
variant I306A/G236S/M237W/F265Y;
variant I306A;
variant M237W;
variant F265Y;
variant I306A/M237W;
variant R130K/M237W;
variant R130K/F265Y;
variant I306A/R130K/M237W;
variant I306A/M237W/F265Y;
variant R130K/M237W/F265Y;
variant G236S/M237W/F265Y;
variant M237W/F265Y;
variant I306A/R130K/M237W/F265Y;
variant R130K/G236S/M237W/F265Y;
variant I306A/R130K/G236S/M237W/F265Y;
variant I306A/R130K/G236S/D259A,
variant I306A/R130K/G236S/M237W/D259A;
variant I306A/R130K/G236S/N258A;
variant I306A/R130K/G236S/M237W/N258A;
variant I306A/R130K/G236S/N258D;
variant I306A/R130K/G236S/M237W/N258D;
variant I306A/R130K/G236S/Q193A;
variant I306A/R130K/G236S/M237W/Q193A;
variant I306A/R130K/G236S/Q193E; and
variant I306A/R130K/G236S/M237W/Q193E.

Preferably, the protein (s) or polypeptide(s) of the present invention result in elevated overall production of short fatty acids, CoA esters of short fatty acids, ethyl esters of short fatty acids, esters of short fatty acids with other metabolites, and/or enzyme bound short fatty acids (C₆ to C₁₂) compared to the wild type polypeptide(s) or the polypeptide(s) without such amino acid substitution(s).

The elevated overall production of short fatty acids, CoA esters of short fatty acids, short fatty acid ethyl esters, short fatty acid esters with other metabolites, and/or enzyme bound short fatty acids (C₆ to C₁₂) is increased at least 2-fold, or preferably at least 5-fold or 10-fold or 20-fold or 27-fold.

The term “short fatty acid” refers to a fatty acid of short to medium length with C₆ to C₁₂.

Variants with Increased Selectivity for C₆

In a preferred embodiment, the polypeptide(s) of the present invention show(s) an increased selectivity for the production of C₆ fatty acids, C₆ CoA esters, C₆ fatty acid ethyl esters, C₆ fatty acid esters with other metabolites, and/or enzyme bound C₆ fatty acids compared to wild type polypeptide(s) or the polypeptide without such amino acid substitution(s).

The selectivity for the production of C₆ fatty acids, C₆ CoA esters, C₆ fatty acid ethyl esters, C₆ fatty acid esters with other metabolites, and/or enzyme bound C₆ fatty acids compared to wild type is increased where the share of C₆ makes up at least 30%, or preferably at least 40% or 50% or 70% or 80% or 90% of the detected chain length between C₆ and C₁₂.

In particular, proteins or polypeptides of the present invention, wherein the amino acid substitution(s) is/are selected from
I306A (in the AT domain on SEQ ID NO: 2) and G236S (in the KS domain on SEQ ID NO: 3);
I306A (in the AT domain on SEQ ID NO: 2) and F265Y (in the KS domain on SEQ ID NO: 3);
I306A (in the AT domain on SEQ ID NO: 2), G236S and M237W (in the KS domain on SEQ ID NO: 3);
I306A (in the AT domain on SEQ ID NO: 2), R130K (in the MPT domain on SEQ ID NO: 1) and G236S (in the KS domain on SEQ ID NO: 3);
G236S and M237W (in the KS domain on SEQ ID NO: 3);
I306A (in the AT domain on SEQ ID NO: 2), R130K (in the MPT domain on SEQ ID NO: 1), G236S and M237W (in the KS domain on SEQ ID NO: 3); R130K (in the MPT domain on SEQ ID NO: 1);
I306A (in the AT domain on SEQ ID NO: 2), G236S and F265Y (in the KS domain on SEQ ID NO: 3);
I306A (in the AT domain on SEQ ID NO: 2), R130K (in the MPT domain on SEQ ID NO: 1) and F265Y (in the KS domain on SEQ ID NO: 3);
I306A (in the AT domain on SEQ ID NO: 2) and R130K (in the MPT domain on SEQ ID NO: 1);
R130K (in the MPT domain on SEQ ID NO: 1) and G236S (in the KS domain on SEQ ID NO: 3);
R130K (in the MPT domain on SEQ ID NO: 1), G236S and M237W (in the KS domain on SEQ ID NO: 3);
G236S and F265Y (in the KS domain on SEQ ID NO: 3);
R130K (in the MPT domain on SEQ ID NO: 1), G236S and F265Y (in the KS domain on SEQ ID NO: 3);
I306A (in the AT domain on SEQ ID NO: 2), R130K (in the MPT domain on SEQ ID NO: 1), G236S and F265Y (in the KS domain on SEQ ID NO: 3);
I306A (in the AT domain on SEQ ID NO: 2), G236S, M237W and F265Y (in the KS domain on SEQ ID NO: 3);
I306A (in the AT domain on SEQ ID NO: 2);
M237W (in the KS domain on SEQ ID NO: 3);
F265Y (in the KS domain on SEQ ID NO: 3);
I306A (in the AT domain on SEQ ID NO: 2) and M237W (in the KS domain on SEQ ID NO: 3);
R130K (in the MPT domain on SEQ ID NO: 1) and M237W (in the KS domain on SEQ ID NO: 3);
R130K (in the MPT domain on SEQ ID NO: 1) and F265Y (in the KS domain on SEQ ID NO: 3);
I306A (in the AT domain on SEQ ID NO: 2), R130K (in the MPT domain on SEQ ID NO: 1) and M237W (in the KS domain on SEQ ID NO: 3);
I306A (in the AT domain on SEQ ID NO: 2), M237W and F265Y (in the KS domain on SEQ ID NO: 3);
R130K (in the MPT domain on SEQ ID NO: 1), M237W and F265Y (in the KS domain on SEQ ID NO: 3);
G236S, M237W and F265Y (in the KS domain on SEQ ID NO: 3);
M237W and F265Y (in the KS domain on SEQ ID NO: 3);
I306A (in the AT domain on SEQ ID NO: 2), R130K (in the MPT domain on SEQ ID NO: 1), M237W and F265Y (in the KS domain on SEQ ID NO: 3);
R130K (in the MPT domain on SEQ ID NO: 1), G236S, M237W and F265Y (in the KS domain on SEQ ID NO: 3);
I306A (in the AT domain on SEQ ID NO: 2), R130K (in the MPT domain on SEQ ID NO: 1), G236S, M237W and F265Y (in the KS domain on SEQ ID NO: 3);
I306A (in the AT domain on SEQ ID NO: 2), R130K (in the MPT domain on SEQ ID NO: 1), G236S and N258A (in the KS domain on SEQ ID NO: 3);
I306A (in the AT domain on SEQ ID NO: 2), R130K (in the MPT domain on SEQ ID NO: 1), G236S and N258D (in the KS domain on SEQ ID NO: 3);
I306A (in the AT domain on SEQ ID NO: 2), R130K (in the MPT domain on SEQ ID NO: 1), G236S, M237W and N258A (in the KS domain on SEQ ID NO: 3);
I306A (in the AT domain on SEQ ID NO: 2), R130K (in the MPT domain on SEQ ID NO: 1), G236S, M237W and N258D (in the KS domain on SEQ ID NO: 3);
   preferably selected from
I306A (in the AT domain on SEQ ID NO: 2) and G236S (in the KS domain on SEQ ID NO: 3);
I306A (in the AT domain on SEQ ID NO: 2) and F265Y (in the KS domain on SEQ ID NO: 3);
I306A (in the AT domain on SEQ ID NO: 2), G236S and M237W (in the KS domain on SEQ ID NO: 3);
I306A (in the AT domain on SEQ ID NO: 2), R130K (in the MPT domain on SEQ ID NO: 1) and G236S (in the KS domain on SEQ ID NO: 3)
G236S and M237W (in the KS domain on SEQ ID NO: 3).
   increase(s) the selectivity for the production of C₆ fatty acids, C₆ CoA, C₆ fatty acid ethyl esters, C₆ fatty acid esters with other metabolites, esters and/or enzyme bound C₆ fatty acids compared to wild type polypeptide(s) or the polypeptide without such amino acid substitution(s).

Namely, proteins or polypeptides of the present invention, wherein the amino acid substitution(s) is/are selected from
I306A and G236S;
I306A and F265Y;
I306A, G236S and M237W;
I306A, R130K and G236S;
G236S and M237W;
I306A, R130K, G236S and M237W;
R130K;
I306A, G236S and F265Y;
I306A, R130K and F265Y;
I306A and R130K;
R130K and G236S;
R130K, G236S and M237W;
G236S and F265Y;
R130K, G236S and F265Y;
I306A, R130K, G236S and F265Y;
I306A, G236S, M237W and F265Y;
I306A;
M237W;
F265Y;
I306A and M237W;
R130K and M237W;
R130K and F265Y;
I306A, R130K and M237W;
I306A, M237W and F265Y;
R130K, M237W and F265Y;
G236S, M237W and F265Y;
M237W and F265Y;
I306A, R130K, M237W and F265Y;
R130K, G236S, M237W and F265Y;
I306A, R130K, G236S, M237W and F265Y;
I306A, R130K, G236S and N258A;
I306A, R130K, G236S and N258D;
I306A, R130K, G236S, M237W and N258A;
I306A, R130K, G236S, M237W and N258D;
   preferably selected from
I306A and G236S;
I306A and F265Y;
I306A, G236S and M237W;
I306A, R130K and G236S;
G236S and M237W;
   more preferably
I306A and G236S,
I306A and F265Y,
I306A, G236S and M237W,
I306A, R130K and G236S,
   increase(s) the selectivity for the production of C₆ fatty acids, C₆ fatty acid CoA esters, C₆ fatty acid ethyl esters, C₆ fatty acid esters with other metabolites, esters and/or enzyme bound C₆ fatty acids compared to wild type polypeptide(s) or the polypeptide without such amino acid substitution(s).
   Variants with Increased Selectivity for C₈

In a preferred embodiment, the protein (s) or polypeptide(s) of the present invention show(s) an increased selectivity for the production of C₈ fatty acids, C₈ fatty acid CoA esters, C₈ fatty acid ethyl esters, C₈ fatty acid esters with other metabolites, and/or enzyme bound C₈ fatty acids compared to wild type polypeptide(s) or the polypeptide without such amino acid substitution(s).

The selectivity for the production of C₈ fatty acids, C₈ fatty acid CoA esters and/or enzyme bound C₈ fatty acids compared to wild type is increased where the share of C₈ makes up at least 30%, or preferably at least 40% or 50% or 70% or 80% or 89% of the detected chain length between C₆ and C₁₂.

In particular, proteins or polypeptides of the present invention, wherein the amino acid substitution(s) is/are selected from
I306A (in the AT domain on SEQ ID NO: 2), R130K (in the MPT domain on SEQ ID NO: 1) and F265Y (in the KS domain on SEQ ID NO: 3)
R130K (in the MPT domain on SEQ ID NO: 1);
I306A (in the AT domain on SEQ ID NO: 2), R130K (in the MPT domain on SEQ ID NO: 1), G236S and M237W (in the KS domain on SEQ ID NO: 3);
R130K (in the MPT domain on SEQ ID NO: 1) and G236S (in the KS domain on SEQ ID NO: 3);
I306A (in the AT domain on SEQ ID NO: 2) and R130K (in the MPT domain on SEQ ID NO: 1);
G236S and M237W (in the KS domain on SEQ ID NO: 3);
I306A (in the AT domain on SEQ ID NO: 2), R130K (in the MPT domain on SEQ ID NO: 1) and G236S (in the KS domain on SEQ ID NO: 3);
I306A (in the AT domain on SEQ ID NO: 2), G236S and M237W (in the KS domain on SEQ ID NO: 3);
R130K (in the MPT domain on SEQ ID NO: 1), G236S and M237W (in the KS domain on SEQ ID NO: 3);
I306A (in the AT domain on SEQ ID NO: 2) and F265Y (in the KS domain on SEQ ID NO: 3);
M237W (in the KS domain on SEQ ID NO: 3);
I306A (in the AT domain on SEQ ID NO: 2), G236S and F265Y (in the KS domain on SEQ ID NO: 3);
G236S and F265Y (in the KS domain on SEQ ID NO: 3);
I306A (in the AT domain on SEQ ID NO: 2) and G236S (in the KS domain on SEQ ID NO: 3);
R130K (in the MPT domain on SEQ ID NO: 1), G236S and F265Y (in the KS domain on SEQ ID NO: 3)
I306A (in the AT domain on SEQ ID NO: 2), R130K (in the MPT domain on SEQ ID NO: 1), G236S and F265Y (in the KS domain on SEQ ID NO: 3);
I306A (in the AT domain on SEQ ID NO: 2), G236S, M237W and F265Y (in the KS domain on SEQ ID NO: 3);
I306A (in the AT domain on SEQ ID NO: 2);
F265Y (in the KS domain on SEQ ID NO: 3);
I306A (in the AT domain on SEQ ID NO: 2) and M237W (in the KS domain on SEQ ID NO: 3);
R130K (in the MPT domain on SEQ ID NO: 1) and M237W (in the KS domain on SEQ ID NO: 3);
R130K (in the MPT domain on SEQ ID NO: 1) and F265Y (in the KS domain on SEQ ID NO: 3);
I306A (in the AT domain on SEQ ID NO: 2), R130K (in the MPT domain on SEQ ID NO: 1) and M237W (in the KS domain on SEQ ID NO: 3);
I306A (in the AT domain on SEQ ID NO: 2), M237W and F265Y (in the KS domain on SEQ ID NO: 3);
R130K (in the MPT domain on SEQ ID NO: 1), M237W and F265Y (in the KS domain on SEQ ID NO: 3);
G236S, M237W and F265Y (in the KS domain on SEQ ID NO: 3);
M237W and F265Y (in the KS domain on SEQ ID NO: 3);
I306A (in the AT domain on SEQ ID NO: 2), R130K (in the MPT domain on SEQ ID NO: 1), M237W and F265Y (in the KS domain on SEQ ID NO: 3);
R130K (in the MPT domain on SEQ ID NO: 1), G236S, M237W and F265Y (in the KS domain on SEQ ID NO: 3);
I306A (in the AT domain on SEQ ID NO: 2), R130K (in the MPT domain on SEQ ID NO: 1), G236S, M237W and F265Y (in the KS domain on SEQ ID NO: 3);
I306A (in the AT domain on SEQ ID NO: 2), R130K (in the MPT domain on SEQ ID NO: 1), G236S and D259A (in the KS domain on SEQ ID NO: 3);
I306A (in the AT domain on SEQ ID NO: 2), R130K (in the MPT domain on SEQ ID NO: 1), G236S, M237W and D259A (in the KS domain on SEQ ID NO: 3);
I306A (in the AT domain on SEQ ID NO: 2), R130K (in the MPT domain on SEQ ID NO: 1), G236S and N258A (in the KS domain on SEQ ID NO: 3);
I306A (in the AT domain on SEQ ID NO: 2), R130K (in the MPT domain on SEQ ID NO: 1), G236S, M237W and N258A (in the KS domain on SEQ ID NO: 3);
I306A (in the AT domain on SEQ ID NO: 2), R130K (in the MPT domain on SEQ ID NO: 1), G236S and N258D (in the KS domain on SEQ ID NO: 3);
I306A (in the AT domain on SEQ ID NO: 2), R130K (in the MPT domain on SEQ ID NO: 1), G236S, M237W and N258D (in the KS domain on SEQ ID NO: 3);
I306A (in the AT domain on SEQ ID NO: 2), R130K (in the MPT domain on SEQ ID NO: 1), G236S and Q193A (in the KS domain on SEQ ID NO: 3);
I306A (in the AT domain on SEQ ID NO: 2), R130K (in the MPT domain on SEQ ID NO: 1), G236S, M237W and Q193A (in the KS domain on SEQ ID NO: 3);
I306A (in the AT domain on SEQ ID NO: 2), R130K (in the MPT domain on SEQ ID NO: 1), G236S and Q193E (in the KS domain on SEQ ID NO: 3);
I306A (in the AT domain on SEQ ID NO: 2), R130K (in the MPT domain on SEQ ID NO: 1), G236S, M237W and Q193E (in the KS domain on SEQ ID NO: 3);
   preferably selected from
I306A (in the AT domain on SEQ ID NO: 2), R130K (in the MPT domain on SEQ ID NO: 1) and F265Y (in the KS domain on SEQ ID NO: 3)
R130K (in the MPT domain on SEQ ID NO: 1);
I306A (in the AT domain on SEQ ID NO: 2), R130K (in the MPT domain on SEQ ID NO: 1), G236S and M237W (in the KS domain on SEQ ID NO: 3);
R130K (in the MPT domain on SEQ ID NO: 1) and G236S (in the KS domain on SEQ ID NO: 3);
I306A (in the AT domain on SEQ ID NO: 2) and R130K (in the MPT domain on SEQ ID NO: 1);
G236S and M237W (in the KS domain on SEQ ID NO: 3);
I306A (in the AT domain on SEQ ID NO: 2), R130K (in the MPT domain on SEQ ID NO: 1) and G236S (in the KS domain on SEQ ID NO: 3);
I306A (in the AT domain on SEQ ID NO: 2), G236S and M237W (in the KS domain on SEQ ID NO: 3);
R130K (in the MPT domain on SEQ ID NO: 1), G236S and M237W (in the KS domain on SEQ ID NO: 3);
   increase(s) the selectivity for the production of C₈ fatty acids, C₈ fatty acid CoA esters, C₈ fatty acid ethyl esters, C₈ fatty acid esters with other metabolites, and/or enzyme bound C₈ fatty acids compared to wild type polypeptide(s) or the polypeptide without such amino acid substitution(s).

Namely, proteins or polypeptides of the present invention, wherein the amino acid substitution(s) is/are selected from
I306A, R130K and F265Y;
R130K;
I306A, R130K, G236S and M237W;
R130K and G236S;
I306A and R130K;
G236S and M237W;
I306A, R130K and G236S;
I306A, G236S and M237W;
R130K, G236S and M237W;
I306A and F265Y;
M237W;
I306A, G236S and F265Y;
G236S and F265Y;
I306A and G236S;
R130K, G236S and F265Y;
I306A, R130K, G236S and F265Y;
I306A, G236S, M237W and F265Y;
I306A;
F265Y;
I306A and M237W;
R130K and M237W;
R130K and F265Y;
I306A, R130K and M237W;
I306A, M237W and F265Y;
R130K, M237W and F265Y;
G236S, M237W and F265Y;
M237W and F265Y;
I306A, R130K, M237W and F265Y;
R130K, G236S, M237W and F265Y;
I306A, R130K, G236S, M237W and F265Y;
I306A, R130K, G236S and D259A,
I306A, R130K, G236S and N258A;
I306A, R130K, G236S and N258D;
I306A, R130K, G236S and Q193A;
I306A, R130K, G236S and Q193E;
I306A, R130K, G236S, M237W and D259A;
I306A, R130K, G236S, M237W and N258A;
I306A, R130K, G236S, M237W and N258D;
I306A, R130K, G236S, M237W and Q193A; and
I306A, R130K, G236S, M237W and Q193E;
   preferably selected from
I306A, R130K and F265Y;
R130K;
I306A, R130K, G236S and M237W;
R130K and G236S;
I306A and R130K;
G236S and M237W
I306A, R130K and G236S;
I306A, G236S and M237W;
R130K, G236S and M237W;
   more preferably selected from
I306A, R130K and F265Y;
R130K;
I306A, R130K, G236S and M237W;
R130K and G236S;
I306A and R130K;
G236S and M237W;
I306A, R130K and G236S;
   increase(s) the selectivity for the production of C₈ fatty acids, C₈ fatty acid CoA esters, C₈ fatty acid ethyl esters, C₈ fatty acid esters with other metabolites, and/or enzyme bound C₈ fatty acids compared to wild type polypeptide(s) or the polypeptide without such amino acid substitution(s).
   Variants with Increased Selectivity for C10 to C12

In a preferred embodiment, the protein (s) or polypeptide(s) of the present invention show(s) an increased selectivity for the production of C₁₀ to C₁₂ fatty acids, C₁₀ to C₁₂ fatty acid CoA esters, C₁₀ to C₁₂ fatty acid ethyl esters, C₁₀ to C₁₂ fatty acid esters with other metabolites, and/or enzyme bound C₁₀ to C₁₂ fatty acids compared to wild type polypeptide(s) or the polypeptide without such amino acid substitution(s).

In particular, proteins or polypeptides of the present invention, wherein the amino acid substitution(s) is/are selected from
I306A (in the AT domain on SEQ ID NO: 2), R130K (in the MPT domain on SEQ ID NO: 1), G236S and M237W (in the KS domain on SEQ ID NO: 3);
G236S and M237W (in the KS domain on SEQ ID NO: 3);
R130K (in the MPT domain on SEQ ID NO: 1);
I306A (in the AT domain on SEQ ID NO: 2), G236S and F265Y (in the KS domain on SEQ ID NO: 3);
I306A (in the AT domain on SEQ ID NO: 2), R130K (in the MPT domain on SEQ ID NO: 1) and G236S (in the KS domain on SEQ ID NO: 3);
R130K (in the MPT domain on SEQ ID NO: 1) and G236S (in the KS domain on SEQ ID NO: 3);
I306A (in the AT domain on SEQ ID NO: 2) and R130K (in the MPT domain on SEQ ID NO: 1);
I306A (in the AT domain on SEQ ID NO: 2), G236S and M237W (in the KS domain on SEQ ID NO: 3);
I306A (in the AT domain on SEQ ID NO: 2) and F265Y (in the KS domain on SEQ ID NO: 3);
M237W (in the KS domain on SEQ ID NO: 3);
I306A (in the AT domain on SEQ ID NO: 2), R130K (in the MPT domain on SEQ ID NO: 1) and F265Y (in the KS domain on SEQ ID NO: 3);
R130K (in the MPT domain on SEQ ID NO: 1), G236S and M237W (in the KS domain on SEQ ID NO: 3);
G236S and F265Y (in the KS domain on SEQ ID NO: 3);
I306A (in the AT domain on SEQ ID NO: 2) and G236S (in the KS domain on SEQ ID NO: 3);
R130K (in the MPT domain on SEQ ID NO: 1), G236S and F265Y (in the KS domain on SEQ ID NO: 3);
I306A (in the AT domain on SEQ ID NO: 2), R130K (in the MPT domain on SEQ ID NO: 1), G236S and F265Y (in the KS domain on SEQ ID NO: 3);
I306A (in the AT domain on SEQ ID NO: 2), G236S, M237W and F265Y (in the KS domain on SEQ ID NO: 3);
I306A (in the AT domain on SEQ ID NO: 2);
F265Y (in the KS domain on SEQ ID NO: 3);
I306A (in the AT domain on SEQ ID NO: 2) and M237W (in the KS domain on SEQ ID NO: 3);
R130K (in the MPT domain on SEQ ID NO: 1) and M237W (in the KS domain on SEQ ID NO: 3);
R130K (in the MPT domain on SEQ ID NO: 1) and F265Y (in the KS domain on SEQ ID NO: 3);
I306A (in the AT domain on SEQ ID NO: 2), R130K (in the MPT domain on SEQ ID NO: 1) and M237W (in the KS domain on SEQ ID NO: 3);
I306A (in the AT domain on SEQ ID NO: 2), M237W and F265Y (in the KS domain on SEQ ID NO: 3);
R130K (in the MPT domain on SEQ ID NO: 1), M237W and F265Y (in the KS domain on SEQ ID NO: 3);
G236S, M237W and F265Y (in the KS domain on SEQ ID NO: 3);
M237W and F265Y (in the KS domain on SEQ ID NO: 3);
I306A (in the AT domain on SEQ ID NO: 2), R130K (in the MPT domain on SEQ ID NO: 1), M237W and F265Y (in the KS domain on SEQ ID NO: 3);
R130K (in the MPT domain on SEQ ID NO: 1), G236S, M237W and F265Y (in the KS domain on SEQ ID NO: 3);
I306A (in the AT domain on SEQ ID NO: 2), R130K (in the MPT domain on SEQ ID NO: 1), G236S, M237W and F265Y (in the KS domain on SEQ ID NO: 3);
I306A (in the AT domain on SEQ ID NO: 2), R130K (in the MPT domain on SEQ ID NO: 1), G236S and D259A (in the KS domain on SEQ ID NO: 3);
I306A (in the AT domain on SEQ ID NO: 2), R130K (in the MPT domain on SEQ ID NO: 1), G236S, M237W and D259A (in the KS domain on SEQ ID NO: 3);
I306A (in the AT domain on SEQ ID NO: 2), R130K (in the MPT domain on SEQ ID NO: 1), G236S and N258A (in the KS domain on SEQ ID NO: 3);
I306A (in the AT domain on SEQ ID NO: 2), R130K (in the MPT domain on SEQ ID NO: 1), G236S, M237W and N258A (in the KS domain on SEQ ID NO: 3);
I306A (in the AT domain on SEQ ID NO: 2), R130K (in the MPT domain on SEQ ID NO: 1), G236S and N258D (in the KS domain on SEQ ID NO: 3);
I306A (in the AT domain on SEQ ID NO: 2), R130K (in the MPT domain on SEQ ID NO: 1), G236S, M237W and N258D (in the KS domain on SEQ ID NO: 3);
I306A (in the AT domain on SEQ ID NO: 2), R130K (in the MPT domain on SEQ ID NO: 1), G236S and Q193A (in the KS domain on SEQ ID NO: 3);
I306A (in the AT domain on SEQ ID NO: 2), R130K (in the MPT domain on SEQ ID NO: 1), G236S, M237W and Q193A (in the KS domain on SEQ ID NO: 3);
I306A (in the AT domain on SEQ ID NO: 2), R130K (in the MPT domain on SEQ ID NO: 1), G236S and Q193E (in the KS domain on SEQ ID NO: 3);
I306A (in the AT domain on SEQ ID NO: 2), R130K (in the MPT domain on SEQ ID NO: 1), G236S, M237W and Q193E (in the KS domain on SEQ ID NO: 3);
   preferably selected from
I306A (in the AT domain on SEQ ID NO: 2), R130K (in the MPT domain on SEQ ID NO: 1), G236S and M237W (in the KS domain on SEQ ID NO: 3);
G236S and M237W (in the KS domain on SEQ ID NO: 3);
R130K (in the MPT domain on SEQ ID NO: 1);
   increase(s) the selectivity for the production of C₁₀ to C₁₂ fatty acids, C₁₀ to C₁₂ fatty acid CoA esters, C₁₀ to C₁₂ fatty acid ethyl esters, C₁₀ to C₁₂ fatty acid esters with other metabolites, and/or enzyme bound C₁₀ to C₁₂ fatty acids compared to wild type polypeptide(s) or the polypeptide without such amino acid substitution(s).

Namely, proteins or polypeptides of the present invention, wherein the amino acid substitution(s) is/are selected from
I306A, R130K, G236S and M237W;
G236S and M237W;
R130K;
I306A, G236S and F265Y;
I306A, R130K and G236S;
R130K and G236S;
I306A and R130K;
I306A, G236S and M237W;
I306A and F265Y;
I306A, R130K and G236S;
M237W;
I306A, R130K and F265Y;
R130K, G236S and M237W;
G236S and F265Y;
I306A and G236S;
R130K, G236S and F265Y;
I306A, R130K, G236S and F265Y;
I306A, G236S, M237W and F265Y;
I306A;
F265Y;
I306A and M237W;
R130K and M237W;
R130K and F265Y;
I306A, R130K and M237W;
I306A, M237W and F265Y;
R130K, M237W and F265Y;
G236S, M237W and F265Y;
M237W and F265Y;
I306A, R130K, M237W and F265Y;
R130K, G236S, M237W and F265Y;
I306A, R130K, G236S, M237W and F265Y;
I306A, R130K, G236S and D259A,
I306A, R130K, G236S and N258A;
I306A, R130K, G236S and N258D;
I306A, R130K, G236S and Q193A;
I306A, R130K, G236S and Q193E;
I306A, R130K, G236S, M237W and D259A;
I306A, R130K, G236S, M237W and N258A;
I306A, R130K, G236S, M237W and N258D;
I306A, R130K, G236S, M237W and Q193A; and
I306A, R130K, G236S, M237W and Q193E;
   preferably selected from
I306A, R130K, G236S and M237W
G236S and M237W
R130K
   increase(s) the selectivity for the production of C₁₀ to C₁₂ fatty acids, C₁₀ to C₁₂ fatty acid CoA esters, C₁₀ to C₁₂ fatty acid ethyl esters, C₁₀ to C₁₂ fatty acid esters with other metabolites, and/or enzyme bound C₁₀ to C₁₂ fatty acids compared to wild type polypeptide(s) or the polypeptide without such amino acid substitution(s).

### Nucleic Acid Molecules

As discussed above, the present invention provides a nucleic acid molecule, coding for a protein or polypeptide according to the present invention.

As discussed above, the present invention provides nucleic acid molecules, coding for the proteins or polypeptides according to the present invention.

As discussed above, the present invention provides a nucleic acid molecule, coding for a domain or subunit of a polypeptide/protein according to the present invention.

Preferably the nucleic acid molecules of the invention comprise or consist of the nucleic acid sequences of SEQ ID NO: 4 coding for the MPT domain (with polypeptide sequence SEQ ID NO: 1), SEQ ID NO: 5 coding for the AT domain (with polypeptide sequence SEQ ID NO: 2) and/or SEQ ID NO: 6 coding for the KS domain (with polypeptide sequence SEQ ID NO: 3), which comprise the respective nucleotide exchanges which lead to the amino acid substitution(s) of the present invention.

SEQ ID NO. 4 shows the nucleic acid sequence (part of GenBank: M30162.1 and J03936.1, split indicated by *) coding for the MPT domain (SEQ ID NO: 1):

(SEQ ID NO: 4)
TCTATCTTAGACATTGTCATTAACAACCCAGTTAACTTAACAATTCACT
TCGGTGGTGAAAAGGGTAAGAGGATCAGAGAAAACTATTCTGCTATGAT
CTTTGAGACTATCGTGGATGGAAAATTGAAGACTGAAAAAATTTTCAAG
GAAATTAATGAGCACAGTACTTCTTACACATTTAGATCTGAAAAAGGTT
TATTGTCTGCTACTCAATTTACACAACCAGCTTTAACTTTGATGGAAAA
AGCTGCTTTCGAAGACTTGAAATCTAAAGGTTTGATCCCAGCCGATGCT
ACTTTTGCTGGTCACTCTTTAGGTGAGTATGCTGCTTTGGCCTCTTTGG
CTGATGTTATGTCTATCGAATCTTTAGTTGAAGTTGTGTTCTACAGAGG
TATGACTATGCAAGTTGCTGTTCCAAGAGATGAGTTGGGCAGATCCAAC
TATGGTATGATTGCCATTAACCCAGGTAGAGTCGCTGCATCATTCTCTC
AAGAAGCTTTGCAATATGTTGTTGAGAGAGTTGGTAAGAGAACCGGCTG
GTTGGTTGAAATCGTCAACTACAACGTTGAAAACCAACAATATGTTGCA
GCTGGTGATCTAAGAGCTTTAGACACCGTTACCAATGTTCTAAACTTCA
TCAAATTACAAAAAATTGATATTATTGAACTACAAAAGTCCTTATCTTT
GGAAGAAGTTGAAGGTCATTTGTTTGAGATCATTGACGAAGCTTCCAAG
AAATCTGCTGTCAAGCCTCGCCCACTTAAATTGGAGAGAGGTTTTGCTT
GTATCCCATTAGTTGGTATTTCTGTTCCTTTCCATTCCACCTACTTGAT
GAATGGTGTTAAACCATTCAAGAGTTTCTTGAAGAAGAATATCATAAAA
GAAAATGTGAAGGTTGCTAGATTGGCCGGAAAGTACATTCCAAACTTGA
CTGCAAAACCATTCCAGGTTACTAAGGAATATTTCCAGGACGTTTATGA
TTTGACTGGCTCCGAACCTATCAAGGAAATCATCGACAACTGGGAAAAG
TATGAACAATCCATGAAGCCGGAAGTTGAGCAAGAATTAGCTCATATTT
TGCTAACTGAATTGTTAGCTTATCAATTTGCCTCTCCTGTGAGATGGAT
TGAAACTCAAGATGTTTTTITGAAGGATTTTAACACTGAAAGGGTTGTT
GAAATCGGTCCTTCTCCAACTTTGGCTGGGATGGCTCAAAGAACCTTGA
ATAAATACGAATCTTACAAGGATGCTGCTCTGTCTTTACATAGAGAAAT
CTTATGCTATTCG

SEQ ID NO: 5 shows the nucleic acid sequence (part of GenBank: M30162.1) coding for the AT domain (SEQ ID NO: 2)

ATGGACGCTTACTCCACAAGACCATTAACCCTATCTCACGGTTCTTTAG
AGCACGTGCTTCTGGTACCAACCGCTTCATTTTTCATTGCTTCGCAATT
ACAAGAACAATTTAATAAAATTTTGCCCGAACCCACTGAAGGGTTTGCT
GCAGATGACGAGCCTACCACACCTGCTGAACTAGTGGGGAAATTCCTTG
GCTACGTATCTTCTCTAGTCGAACCTTCCAAGGTCGGTCAATTCGATCA
GGTCTTGAACCTTTGCTTAACAGAATTTGAAAACTGTTATTTAGAAGGC
AATGACATTCACGCCTTGGCTGCTAAACTATTACAGGAAAACGACACAA
CTTTAGTGAAGACTAAAGAACTAATTAAAAATTATATTACCGCCAGAAT
AATGGCTAAGAGACCATTTGACAAAAAATCCAACTCTGCTCTTTTTAGG
GCCGTCGGCGAGGGTAACGCACAATTGGTAGCCATTTTCGGTGGTCAAG
GTAACACCGACGACTACTTTGAAGAATTGCGTGATCTATATCAAACTTA
TCATGTCTTAGTGGGAGATTTAATCAAGTTCTCCGCTGAAACTTTAAGT
GAACTGATTAGAACTACTTTAGATGCTGAAAAAGTCTTTACTCAAGGTT
TAAACATATTGGAATGGTTGGAGAACCCTTCAAATACCCCAGACAAGGA
CTATTTACTTTCCATTCCAATTTCATGCCCCTTAATTGGTGTCATTCAA
TTGGCTCACTACGTAGTTACTGCCAAGCTTTTGGGTTTCACTCCAGGTG
AGTTAAGATCTTACTTAAAAGGTGCTACAGGTCACTCTCAAGGTTTGGT
TACTGCTGTCGCCATAGCTGAGACGGATTCCTGGGAATCCTTCTTCGTC
TCCGTAAGAAAAGCAATTACTGTATTATTCTTCATCGGTGTTCGTTGTT
ACGAAGCATACCCAAACACTTCCCTACCACCATCCATCTTGGAAGATTC
CTTGGAAAACAATGAAGGTGTTCCATCTCCAATGTTGTCCATTTCCAAT
CTAACTCAAGAACAAGTTCAAGACTATGTAAATAAGACTAACTCTCATT
TGCCAGCTGGTAAACAAGTTGAAATTTCTCTAGTCAATGGTGCGAAGAA
TCTAGTCGTATCGGGCCCACCACAATCATTATATGGTTTAAACTTGACT
TTAAGAAAGGCCAAGGCCCCATCTGGACTGGATCAATCAAGAATCCCAT
TCAGCGAAAGAAAATTGAAGTTCTCCAATAGGTTCTTACCTGTTGCATC
ACCATTCCATTCCCATCTATTGGTTCCAGCTTCAGATTTGATTAACAAA
GACTTAGTCAAAAACAATGTCAGCTTTAACGCTAAAGATATTCAAATCC
CCGTTTACGACACTTTTGATGGTTCAGATCTAAGAGTCCTTTCAGGTTC
CATTTCCGAGAGAATCGTCGACTGCATCATTAGATTACCTGTCAAATGG
GAAACTACTACACAATTCAAAGCCACCCACATATTAGACTTTGGTCCAG
GTGGAGCTTCCGGTTTAGGTGTTTTAACCCATCGTAATAAAGATGGTAC
TGGTGTTCGTGTTATCGTTGCCGGTACTCTCGACATTAACCCAGATGAT
GATTACGGATTCAAGCAAGAAATCTTTGATGTTACT

SEQ ID NO: 6 shows the nucleic acid sequence (part of GenBank: J03936.1) coding for the KS domain (SEQ ID NO: 3)

TTGGAAAGAGTTATTGTAGTTACCGGTTTTGCTGAAGTCGGCCCATGGG
GTTCGGCCAGAACAAGATGGGAAATGGAAGCTTTTGGTGAATTTTCGTT
GGAAGGTTGCGTTGAAATGGCCTGGATTATGGGCTTCATTTCATACCAT
AACGGTAATTTGAAGGGTCGTCCATACACTGGTTGGGTTGATTCCAAAA
CAAAAGAACCAGTTGATGACAAGGACGTTAAGGCCAAGTATGAAACATC
AATCCTAGAACACAGTGGTATCAGATTGATCGAACCAGAGTTATTCAAT
GGTTACAACCCAGAAAAGAAGGAAATGATTCAAGAAGTCATTGTCGAAG
AAGACTTGGAACCATTTGAGGCTTCGAAGGAAACTGCCGAACAATTTAA
ACACCAACATGGTGACAAAGTGGATATCTTCGAAATCCCAGAAACAGGA
GAGTACTCTGTTAAGTTACTAAAGGGTGCCACTTTATACATTCCAAAGG
CTTTGAGATTTGACCGTTTGGTTGCAGGTCAAATTCCAACTGGTTGGAA
TGCTAAGACTTATGGTATCTCTGATGATATCATTTCTCAGGTTGACCCA
ATCACATTATTCGTTTTGGTCTCTGTTGTGGAAGCATTTATTGCATCTG
GTATCACCGACCCATACGAAATGTACAAATACGTACATGTTTCTGAGGT
TGGTAACTGTTCTGGTTCTGGTATGGGTGGTGTTTCTGCCTTACGTGGT
ATGTTTAAGGACCGTTTCAAGGATGAGCCTGTCCAAAATGATATTTTAC
AAGAATCATTTATCAACACCATGTCCGCTTGGGTTAATATGTTGTTGAT
TTCCTCATCTGGTCCAATCAAGACACCTGTTGGTGCCTGTGCCACATCC
GTGGAATCTGTTGACATTGGTGTAGAAACCATCTTGTCTGGTAAGGCTA
GAATCTGTATTGTCGGTGGTTACGATGATTTCCAAGAAGAAGGCTCCTT
TGAGTTCGGTAACATGAAGGCCACTTCCAACACTTTGGAAGAATTTGAA
CATGGTCGTACCCCAGCGGAAATGTCCAGACCTGCCACCACTACCCGTA
ACGGTTTTATGGAAGCTCAAGGTGCTGGTATTCAAATCATCATGCAAGC
TGATTTAGCTTTGAAGATGGGTGTGCCAATTTACGGTATTGTTGCCATG
GCTGCTACCGCCACCCATAAGATTGGTAGATCTGTGCCAGCTCCAGGTA
AGGGTATTTTAACCACTGCTCGTGAACACCACTCCAGTGTTAAGTATGC
TTCACCAAACTTGAACATGAAGTACAGAAAGCGCCAATTGGTTACTCGT
GAAGCTCAGATTAAAGATTGGGTAGAAAACGAATTGGAAGCTTTGAAGT
TGGAGGCCGAAGAAATTCCAAGCGAAGACCAAAACGAGTTCTTACTTGA
ACGTACCAGAGAAATCCACAACGAAGCTGAAAGTCAATTGAGAGCTGCA
CAACAACAATGGGGTAACGACTTCTACAAGAGGGACCCACGTATTGCTC
CATTGAGAGGAGCACTGGCTACTTACGGTTTAACTATTGATGACTTGGG
TGTCGCTTCATTCCACGGTACATCCACAAAGGCTAATGACAAGAACGAA
TCTGCCACAATTAATGAAATGATGAAGCATTTGGGTAGATCTGAAGGTA
ATCCCGTCATTGGTGTTTTCCAAAAGTTCTTGACTGGTCATCCAAAGGG
TGCTGCTGGTGCATGGATGATGAATGGTGCTTTGCAAATTCTAAACAGT
GGTATTATTCCAGGTAACCGTAACGCTGATAACGTGGATAAGATCTTGG
AGCAATTTGAATACGTCTTGTACCCATCCAAGACTTTAAAGACCGACGG
TGTCAGAGCCGTGTCCATCACTTCTTTCGGTTTTGGTCAAAAGGGTGGT
CAAGCTATTGTGGTTCATCCAGACTACTTATACGGTGCT

In one embodiment, the nucleic acid molecule of the present invention further comprises:
vector nucleic acid sequences, preferably expression vector sequences, and/or
promoter nucleic acid sequences and terminator nucleic acid sequences, and/or
comprises other regulatory nucleic acid sequence.

In one embodiment, the nucleic acid molecule of the present invention comprises dsDNA, ssDNA, cDNA, LNA, PNA, CNA, RNA or mRNA or combinations thereof.

The nucleic acid molecules according to the invention preferably comprise nucleic acid sequences, which are (except for the addition of the amino acid substitution(s) according to the invention) identical with the naturally occurring nucleic acid sequence or are codon-optimized for the use in a host cell.

The nucleic acid molecule used according to the present invention is preferably a nucleic acid expression construct.

Nucleic acid expression constructs according to the invention are expression cassettes comprising a nucleic acid molecule according to the invention, or expression vectors comprising a nucleic acid molecule according to the invention or an expression cassette, for example.

A nucleic acid expression construct preferably comprises regulatory sequences, such as promoter and terminator sequences, which are operatively linked with the nucleic acid sequence coding for the polypeptide(s) of the invention.

The nucleic acid expression construct may further comprise 5′ and/or 3′ recognition sequences and/or selection markers.

### Host Cells

As discussed above, the present invention provides host cells containing a nucleic acid molecule according to the present invention.

Preferably, the host cells of the present invention express said nucleic acid molecule.

Preferably, a host cell according to the present invention is a bacterial cell.

The bacterial cell is more preferably a member of a genus selected from the group Corynebacterium, Mycobacterium, Escherichia, Nocordia, Bacillus, Clostridium, Pseudomonas, Lactobacillus or Leuconostoc.

The bacterial cell is more preferably a member of a species selected from the group of Corynebacterium glutamicum, Escherichia coli, Bacillus subtilis; Clostridium ljungdahlii, Pseudomonas putida; Lactobacillus bifermentans or Leuconostoc mesenteroides.

Preferably, a host cell according to the present invention is a fungus cell and more preferably a yeast cell.

The yeast cell is preferably a member of a genus selected from the group of Saccharomyces species, Kluyveromyces sp., Hansenula sp., Arxula sp., Rhodosporidium sp., Pichia sp. or Yarrowia sp.

The yeast cell is more preferably a member of a species selected from the group of S. cerevisiae, S. bulderi, S. barnetti, S. exiguus, S. uvarum, S. diastaticus, K lactis, K. marxianus, K fragilis, H. polymorpha, P. pastoris and Y. lipolytica, such as S. cerevisiae, K. lactis, H. polymorpha, P. pastoris, K. marxianus, or Y. lipolytica.

In a preferred embodiment, the host cell belongs to the species Saccharomyces cerevisiae.

Preferably, a host cell according to the present invention is an algae cell.

The algae cell is more preferably a member of a genus selected from the group Chlamydomonas, Chlorella, Haematococcus, Dunaliella, Nannochloropsis, Thalassiosira, Phaeodactylum, Porphyridium or Scenedesmus The algae cell is more preferably a member of a species selected from the group of Chlamydomonas reinhardtii or Haematococcus pluvialis.

Preferably, the host cell (preferably yeast cell) has an elevated overall production of short fatty acids, CoA esters of short fatty acids, ethyl esters of short fatty acids, esters of short fatty acids with other metabolites, and/or enzyme bound short fatty acids (C₆ to C₁₂) compared to a cell not containing a nucleic acid molecule according to the present invention.

In a preferred embodiment, the host cell has an increased yield or increased selectivity in the production of C₆ fatty acids, C₆ fatty acid CoA esters, C₆ fatty acid ethyl esters, C₆ fatty acid esters with other metabolites, and/or enzyme bound C₆ fatty acids compared to a cell not containing a nucleic acid molecule according to the present invention.

The selectivity for the production of C₆ fatty acids, C₆ CoA esters and/or enzyme bound C₆ fatty acids compared to wild type is increased where the share of C₆ makes up at least 30%, or preferably at least 40% or 50% or 70% or 80% or 90% of the detected chain length between C₆ and C₁₂.

As discussed above, in particular proteins or polypeptides of the present invention, wherein the amino acid substitution(s) is/are selected from
I306A (in the AT domain on SEQ ID NO: 2) and G236S (in the KS domain on SEQ ID NO: 3);
I306A (in the AT domain on SEQ ID NO: 2) and F265Y (in the KS domain on SEQ ID NO: 3);
I306A (in the AT domain on SEQ ID NO: 2), G236S and M237W (in the KS domain on SEQ ID NO: 3);
I306A (in the AT domain on SEQ ID NO: 2), R130K (in the MPT domain on SEQ ID NO: 1) and G236S (in the KS domain on SEQ ID NO: 3);
G236S and M237W (in the KS domain on SEQ ID NO: 3);
I306A (in the AT domain on SEQ ID NO: 2), R130K (in the MPT domain on SEQ ID NO: 1), G236S and M237W (in the KS domain on SEQ ID NO: 3);
R130K (in the MPT domain on SEQ ID NO: 1);
I306A (in the AT domain on SEQ ID NO: 2), G236S and F265Y (in the KS domain on SEQ ID NO: 3);
I306A (in the AT domain on SEQ ID NO: 2), R130K (in the MPT domain on SEQ ID NO: 1) and F265Y (in the KS domain on SEQ ID NO: 3);
I306A (in the AT domain on SEQ ID NO: 2) and R130K (in the MPT domain on SEQ ID NO: 1);
R130K (in the MPT domain on SEQ ID NO: 1) and G236S (in the KS domain on SEQ ID NO: 3);
R130K (in the MPT domain on SEQ ID NO: 1), G236S and M237W (in the KS domain on SEQ ID NO: 3);
G236S and F265Y (in the KS domain on SEQ ID NO: 3);
R130K (in the MPT domain on SEQ ID NO: 1), G236S and F265Y (in the KS domain on SEQ ID NO: 3);
I306A (in the AT domain on SEQ ID NO: 2), R130K (in the MPT domain on SEQ ID NO: 1), G236S and F265Y (in the KS domain on SEQ ID NO: 3);
I306A (in the AT domain on SEQ ID NO: 2), G236S, M237W and F265Y (in the KS domain on SEQ ID NO: 3);
I306A (in the AT domain on SEQ ID NO: 2);
M237W (in the KS domain on SEQ ID NO: 3);
F265Y (in the KS domain on SEQ ID NO: 3);
I306A (in the AT domain on SEQ ID NO: 2) and M237W (in the KS domain on SEQ ID NO: 3);
R130K (in the MPT domain on SEQ ID NO: 1) and M237W (in the KS domain on SEQ ID NO: 3);
R130K (in the MPT domain on SEQ ID NO: 1) and F265Y (in the KS domain on SEQ ID NO: 3);
I306A (in the AT domain on SEQ ID NO: 2), R130K (in the MPT domain on SEQ ID NO: 1) and M237W (in the KS domain on SEQ ID NO: 3);
I306A (in the AT domain on SEQ ID NO: 2), M237W and F265Y (in the KS domain on SEQ ID NO: 3);
R130K (in the MPT domain on SEQ ID NO: 1), M237W and F265Y (in the KS domain on SEQ ID NO: 3);
G236S, M237W and F265Y (in the KS domain on SEQ ID NO: 3);
M237W and F265Y (in the KS domain on SEQ ID NO: 3);
I306A (in the AT domain on SEQ ID NO: 2), R130K (in the MPT domain on SEQ ID NO: 1), M237W and F265Y (in the KS domain on SEQ ID NO: 3);
R130K (in the MPT domain on SEQ ID NO: 1), G236S, M237W and F265Y (in the KS domain on SEQ ID NO: 3);
I306A (in the AT domain on SEQ ID NO: 2), R130K (in the MPT domain on SEQ ID NO: 1), G236S, M237W and F265Y (in the KS domain on SEQ ID NO: 3);
I306A (in the AT domain on SEQ ID NO: 2), R130K (in the MPT domain on SEQ ID NO: 1), G236S and N258A (in the KS domain on SEQ ID NO: 3);
I306A (in the AT domain on SEQ ID NO: 2), R130K (in the MPT domain on SEQ ID NO: 1), G236S and N258D (in the KS domain on SEQ ID NO: 3);
I306A (in the AT domain on SEQ ID NO: 2), R130K (in the MPT domain on SEQ ID NO: 1), G236S, M237W and N258A (in the KS domain on SEQ ID NO: 3);
I306A (in the AT domain on SEQ ID NO: 2), R130K (in the MPT domain on SEQ ID NO: 1), G236S, M237W and N258D (in the KS domain on SEQ ID NO: 3);
   preferably selected from
I306A (in the AT domain on SEQ ID NO: 2) and G236S (in the KS domain on SEQ ID NO: 3);
I306A (in the AT domain on SEQ ID NO: 2) and F265Y (in the KS domain on SEQ ID NO: 3);
I306A (in the AT domain on SEQ ID NO: 2), G236S and M237W (in the KS domain on SEQ ID NO: 3);
I306A (in the AT domain on SEQ ID NO: 2), R130K (in the MPT domain on SEQ ID NO: 1) and G236S (in the KS domain on SEQ ID NO: 3)
G236S and M237W (in the KS domain on SEQ ID NO: 3),
   more preferably
I306A and G236S,
I306A and F265Y,
I306A, G236S and M237W,
I306A, R130K and G236S,
   increase(s) the selectivity for the production of C₆ fatty acids, C₆ CoA, C₆ fatty acid ethyl esters, C₆ fatty acid esters with other metabolites, esters and/or enzyme bound C₆ fatty acids compared to wild type polypeptide(s) or the polypeptide without such amino acid substitution(s).

In a preferred embodiment, the host cell has an increased yield or increased selectivity in the production of C₈ fatty acids, C₈ fatty acid CoA esters, C₈ fatty acid ethyl esters, C₈ fatty acid esters with other metabolites, and/or enzyme bound C₈ fatty acids compared to a cell not containing a nucleic acid molecule according to the present invention.

The selectivity for the production of C₈ fatty acids, C₈ CoA esters and/or enzyme bound C₈ fatty acids compared to wild type is increased where the share of C₈ makes up at least 30%, or preferably at least 40% or 50% or 70% or 80% or 89% of the detected chain length between C₆ and C₁₂.

As discussed above, in particular proteins or polypeptides of the present invention, wherein the amino acid substitution(s) is/are selected from
I306A (in the AT domain on SEQ ID NO: 2), R130K (in the MPT domain on SEQ ID NO: 1) and F265Y (in the KS domain on SEQ ID NO: 3)
R130K (in the MPT domain on SEQ ID NO: 1);
I306A (in the AT domain on SEQ ID NO: 2), R130K (in the MPT domain on SEQ ID NO: 1), G236S and M237W (in the KS domain on SEQ ID NO: 3);
R130K (in the MPT domain on SEQ ID NO: 1) and G236S (in the KS domain on SEQ ID NO: 3);
I306A (in the AT domain on SEQ ID NO: 2) and R130K (in the MPT domain on SEQ ID NO: 1);
G236S and M237W (in the KS domain on SEQ ID NO: 3);
I306A (in the AT domain on SEQ ID NO: 2), R130K (in the MPT domain on SEQ ID NO: 1) and G236S (in the KS domain on SEQ ID NO: 3);
I306A (in the AT domain on SEQ ID NO: 2), G236S and M237W (in the KS domain on SEQ ID NO: 3);
R130K (in the MPT domain on SEQ ID NO: 1), G236S and M237W (in the KS domain on SEQ ID NO: 3);
I306A (in the AT domain on SEQ ID NO: 2) and F265Y (in the KS domain on SEQ ID NO: 3);
M237W (in the KS domain on SEQ ID NO: 3);
I306A (in the AT domain on SEQ ID NO: 2), G236S and F265Y (in the KS domain on SEQ ID NO: 3);
G236S and F265Y (in the KS domain on SEQ ID NO: 3);
I306A (in the AT domain on SEQ ID NO: 2) and G236S (in the KS domain on SEQ ID NO: 3);
R130K (in the MPT domain on SEQ ID NO: 1), G236S and F265Y (in the KS domain on SEQ ID NO: 3)
I306A (in the AT domain on SEQ ID NO: 2), R130K (in the MPT domain on SEQ ID NO: 1), G236S and F265Y (in the KS domain on SEQ ID NO: 3);
I306A (in the AT domain on SEQ ID NO: 2), G236S, M237W and F265Y (in the KS domain on SEQ ID NO: 3);
I306A (in the AT domain on SEQ ID NO: 2);
F265Y (in the KS domain on SEQ ID NO: 3);
I306A (in the AT domain on SEQ ID NO: 2) and M237W (in the KS domain on SEQ ID NO: 3);
R130K (in the MPT domain on SEQ ID NO: 1) and M237W (in the KS domain on SEQ ID NO: 3);
R130K (in the MPT domain on SEQ ID NO: 1) and F265Y (in the KS domain on SEQ ID NO: 3);
I306A (in the AT domain on SEQ ID NO: 2), R130K (in the MPT domain on SEQ ID NO: 1) and M237W (in the KS domain on SEQ ID NO: 3);
I306A (in the AT domain on SEQ ID NO: 2), M237W and F265Y (in the KS domain on SEQ ID NO: 3);
R130K (in the MPT domain on SEQ ID NO: 1), M237W and F265Y (in the KS domain on SEQ ID NO: 3);
G236S, M237W and F265Y (in the KS domain on SEQ ID NO: 3);
M237W and F265Y (in the KS domain on SEQ ID NO: 3);
I306A (in the AT domain on SEQ ID NO: 2), R130K (in the MPT domain on SEQ ID NO: 1), M237W and F265Y (in the KS domain on SEQ ID NO: 3);
R130K (in the MPT domain on SEQ ID NO: 1), G236S, M237W and F265Y (in the KS domain on SEQ ID NO: 3);
I306A (in the AT domain on SEQ ID NO: 2), R130K (in the MPT domain on SEQ ID NO: 1), G236S, M237W and F265Y (in the KS domain on SEQ ID NO: 3);
I306A (in the AT domain on SEQ ID NO: 2), R130K (in the MPT domain on SEQ ID NO: 1), G236S and D259A (in the KS domain on SEQ ID NO: 3);
I306A (in the AT domain on SEQ ID NO: 2), R130K (in the MPT domain on SEQ ID NO: 1), G236S, M237W and D259A (in the KS domain on SEQ ID NO: 3);
I306A (in the AT domain on SEQ ID NO: 2), R130K (in the MPT domain on SEQ ID NO: 1), G236S and N258A (in the KS domain on SEQ ID NO: 3);
I306A (in the AT domain on SEQ ID NO: 2), R130K (in the MPT domain on SEQ ID NO: 1), G236S, M237W and N258A (in the KS domain on SEQ ID NO: 3);
I306A (in the AT domain on SEQ ID NO: 2), R130K (in the MPT domain on SEQ ID NO: 1), G236S and N258D (in the KS domain on SEQ ID NO: 3);
I306A (in the AT domain on SEQ ID NO: 2), R130K (in the MPT domain on SEQ ID NO: 1), G236S, M237W and N258D (in the KS domain on SEQ ID NO: 3);
I306A (in the AT domain on SEQ ID NO: 2), R130K (in the MPT domain on SEQ ID NO: 1), G236S and Q193A (in the KS domain on SEQ ID NO: 3);
I306A (in the AT domain on SEQ ID NO: 2), R130K (in the MPT domain on SEQ ID NO: 1), G236S, M237W and Q193A (in the KS domain on SEQ ID NO: 3);
I306A (in the AT domain on SEQ ID NO: 2), R130K (in the MPT domain on SEQ ID NO: 1), G236S and Q193E (in the KS domain on SEQ ID NO: 3);
I306A (in the AT domain on SEQ ID NO: 2), R130K (in the MPT domain on SEQ ID NO: 1), G236S, M237W and Q193E (in the KS domain on SEQ ID NO: 3);
   preferably selected from
I306A (in the AT domain on SEQ ID NO: 2), R130K (in the MPT domain on SEQ ID NO: 1) and F265Y (in the KS domain on SEQ ID NO: 3)
R130K (in the MPT domain on SEQ ID NO: 1);
I306A (in the AT domain on SEQ ID NO: 2), R130K (in the MPT domain on SEQ ID NO: 1), G236S and M237W (in the KS domain on SEQ ID NO: 3);
R130K (in the MPT domain on SEQ ID NO: 1) and G236S (in the KS domain on SEQ ID NO: 3);
I306A (in the AT domain on SEQ ID NO: 2) and R130K (in the MPT domain on SEQ ID NO: 1);
G236S and M237W (in the KS domain on SEQ ID NO: 3);
I306A (in the AT domain on SEQ ID NO: 2), R130K (in the MPT domain on SEQ ID NO: 1) and G236S (in the KS domain on SEQ ID NO: 3);
I306A (in the AT domain on SEQ ID NO: 2), G236S and M237W (in the KS domain on SEQ ID NO: 3);
R130K (in the MPT domain on SEQ ID NO: 1), G236S and M237W (in the KS domain on SEQ ID NO: 3);
   more preferably,
I306A, R130K and F265Y;
R130K;
I306A, R130K, G236S and M237W;
R130K and G236S;
I306A and R130K;
G236S and M237W;
I306A, R130K and G236S;
   increase(s) the selectivity for the production of C₈ fatty acids, C₈ fatty acid CoA esters, C₈ fatty acid ethyl esters, C₈ fatty acid esters with other metabolites, and/or enzyme bound C₈ fatty acids compared to wild type polypeptide(s) or the polypeptide without such amino acid substitution(s).

In a preferred embodiment, the host cell has an increased yield or increased selectivity in the production of C₁₀ to C₁₂ fatty acids, C₁₀ to C₁₂ fatty acid CoA esters, C₁₀ to C₁₂ fatty acid ethyl esters, C₁₀ to C₁₂ fatty acid esters with other metabolites, and/or enzyme bound C₁₀ to C₁₂ fatty acids compared to a cell not containing a nucleic acid molecule according to the present invention.

As discussed above, in particular proteins or polypeptides of the present invention, wherein the amino acid substitution(s) is/are selected from
I306A (in the AT domain on SEQ ID NO: 2), R130K (in the MPT domain on SEQ ID NO: 1), G236S and M237W (in the KS domain on SEQ ID NO: 3);
G236S and M237W (in the KS domain on SEQ ID NO: 3);
R130K (in the MPT domain on SEQ ID NO: 1);
I306A (in the AT domain on SEQ ID NO: 2), G236S and F265Y (in the KS domain on SEQ ID NO: 3);
I306A (in the AT domain on SEQ ID NO: 2), R130K (in the MPT domain on SEQ ID NO: 1) and G236S (in the KS domain on SEQ ID NO: 3);
R130K (in the MPT domain on SEQ ID NO: 1) and G236S (in the KS domain on SEQ ID NO: 3);
I306A (in the AT domain on SEQ ID NO: 2) and R130K (in the MPT domain on SEQ ID NO: 1);
I306A (in the AT domain on SEQ ID NO: 2), G236S and M237W (in the KS domain on SEQ ID NO: 3);
I306A (in the AT domain on SEQ ID NO: 2) and F265Y (in the KS domain on SEQ ID NO: 3);
M237W (in the KS domain on SEQ ID NO: 3);
I306A (in the AT domain on SEQ ID NO: 2), R130K (in the MPT domain on SEQ ID NO: 1) and F265Y (in the KS domain on SEQ ID NO: 3);
R130K (in the MPT domain on SEQ ID NO: 1), G236S and M237W (in the KS domain on SEQ ID NO: 3);
G236S and F265Y (in the KS domain on SEQ ID NO: 3);
I306A (in the AT domain on SEQ ID NO: 2) and G236S (in the KS domain on SEQ ID NO: 3);
R130K (in the MPT domain on SEQ ID NO: 1), G236S and F265Y (in the KS domain on SEQ ID NO: 3);
I306A (in the AT domain on SEQ ID NO: 2), R130K (in the MPT domain on SEQ ID NO: 1), G236S and F265Y (in the KS domain on SEQ ID NO: 3);
I306A (in the AT domain on SEQ ID NO: 2), G236S, M237W and F265Y (in the KS domain on SEQ ID NO: 3);
I306A (in the AT domain on SEQ ID NO: 2);
F265Y (in the KS domain on SEQ ID NO: 3);
I306A (in the AT domain on SEQ ID NO: 2) and M237W (in the KS domain on SEQ ID NO: 3);
R130K (in the MPT domain on SEQ ID NO: 1) and M237W (in the KS domain on SEQ ID NO: 3);
R130K (in the MPT domain on SEQ ID NO: 1) and F265Y (in the KS domain on SEQ ID NO: 3);
I306A (in the AT domain on SEQ ID NO: 2), R130K (in the MPT domain on SEQ ID NO: 1) and M237W (in the KS domain on SEQ ID NO: 3);
I306A (in the AT domain on SEQ ID NO: 2), M237W and F265Y (in the KS domain on SEQ ID NO: 3);
R130K (in the MPT domain on SEQ ID NO: 1), M237W and F265Y (in the KS domain on SEQ ID NO: 3);
G236S, M237W and F265Y (in the KS domain on SEQ ID NO: 3);
M237W and F265Y (in the KS domain on SEQ ID NO: 3);
I306A (in the AT domain on SEQ ID NO: 2), R130K (in the MPT domain on SEQ ID NO: 1), M237W and F265Y (in the KS domain on SEQ ID NO: 3);
R130K (in the MPT domain on SEQ ID NO: 1), G236S, M237W and F265Y (in the KS domain on SEQ ID NO: 3);
I306A (in the AT domain on SEQ ID NO: 2), R130K (in the MPT domain on SEQ ID NO: 1), G236S, M237W and F265Y (in the KS domain on SEQ ID NO: 3);
I306A (in the AT domain on SEQ ID NO: 2), R130K (in the MPT domain on SEQ ID NO: 1), G236S and D259A (in the KS domain on SEQ ID NO: 3);
I306A (in the AT domain on SEQ ID NO: 2), R130K (in the MPT domain on SEQ ID NO: 1), G236S, M237W and D259A (in the KS domain on SEQ ID NO: 3);
I306A (in the AT domain on SEQ ID NO: 2), R130K (in the MPT domain on SEQ ID NO: 1), G236S and N258A (in the KS domain on SEQ ID NO: 3);
I306A (in the AT domain on SEQ ID NO: 2), R130K (in the MPT domain on SEQ ID NO: 1), G236S, M237W and N258A (in the KS domain on SEQ ID NO: 3);
I306A (in the AT domain on SEQ ID NO: 2), R130K (in the MPT domain on SEQ ID NO: 1), G236S and N258D (in the KS domain on SEQ ID NO: 3);
I306A (in the AT domain on SEQ ID NO: 2), R130K (in the MPT domain on SEQ ID NO: 1), G236S, M237W and N258D (in the KS domain on SEQ ID NO: 3);
I306A (in the AT domain on SEQ ID NO: 2), R130K (in the MPT domain on SEQ ID NO: 1), G236S and Q193A (in the KS domain on SEQ ID NO: 3);
I306A (in the AT domain on SEQ ID NO: 2), R130K (in the MPT domain on SEQ ID NO: 1), G236S, M237W and Q193A (in the KS domain on SEQ ID NO: 3);
I306A (in the AT domain on SEQ ID NO: 2), R130K (in the MPT domain on SEQ ID NO: 1), G236S and Q193E (in the KS domain on SEQ ID NO: 3);
I306A (in the AT domain on SEQ ID NO: 2), R130K (in the MPT domain on SEQ ID NO: 1), G236S, M237W and Q193E (in the KS domain on SEQ ID NO: 3);
   preferably selected from
I306A (in the AT domain on SEQ ID NO: 2), R130K (in the MPT domain on SEQ ID NO: 1), G236S and M237W (in the KS domain on SEQ ID NO: 3);
G236S and M237W (in the KS domain on SEQ ID NO: 3);
R130K (in the MPT domain on SEQ ID NO: 1);
   increase(s) the selectivity for the production of C₁₀ to C₁₂ fatty acids, C₁₀ to C₁₂ fatty acid CoA esters, C₁₀ to C₁₂ fatty acid ethyl esters, C₁₀ to C₁₂ fatty acid esters with other metabolites, and/or enzyme bound C₁₀ to C₁₂ fatty acids compared to wild type polypeptide(s) or the polypeptide without such amino acid substitution(s).
   Methods and Uses for Producing Fatty Acids and/or Biofuels and/or Flavoring Substances and/or Fine Chemicals

As discussed above, the present invention provides a method for the production of short fatty acids, CoA esters of short fatty acids, short fatty acid ethyl esters, short fatty acid esters with other metabolites, and/or enzyme bound short fatty acids (C₆ to C₁₂).

Said method comprises the expression of a nucleic acid molecule according to the present invention, preferably in a host cell according to the present invention.

As discussed above, the present invention provides a method for the production of
biofuels,
flavoring compounds or substances
and/or
fine chemicals.

Said method comprises the expression of a nucleic acid molecule according to the present invention, preferably in a host cell according to the present invention.

Biofuels which can be produced with the method are, for example, short alkanes, short alkenes, short alkynes, short esters or alcohols.

Flavoring compounds or substances which can be produced with the method are, for example, fatty acids esterified with short alcohols or esters from short fatty acids (C₆ to C₁₂).

Fine chemicals which can be produced with the method are, for example, natural compounds, where preferably short fatty acids (C₆ to C₁₂) or their derivatives (such as CoA esters, methyl/ethyl esters, esters with other metabolites, alcohols) are used as building block(s).

As discussed above, the present invention provides the use of
a protein or polypeptide according to the present invention,
a nucleic acid molecule according to the present invention, or
a host cell according to the present invention,
   for
the bulk production of short fatty acids (C₆ to C₁₂), CoA esters of short fatty acids, short fatty acid ethyl esters, short fatty acid esters with other metabolites, enzyme bound short fatty acids; or
the specific production of short fatty acids (C₆ or C₈), C₆-CoA esters or C₈-CoA esters, fatty acid (C₆ or C₈) ethyl esters, fatty acid (C₆ or C₈) esters with other metabolites, enzyme bound short fatty acids (C₆ or C₈).

In particular, the present invention provides the use of a protein or polypeptide according to the present invention, a nucleic acid molecule according to the present invention, or a host cell according to the present invention, for
the bulk production of short fatty acids (C₆ to C₁₂),
the specific production of C₆ fatty acids,
the specific production of C₈ fatty acids,
the bulk production of CoA esters of short fatty acids (C₆ to C₁₂),
the specific production of C₆ fatty acid CoA esters,
the specific production of C₈ fatty acid CoA esters,
the bulk production of ethyl esters of short fatty acids (C₆ to C₁₂),
the specific production of C₆ fatty acid ethyl esters,
the specific production of C₈ fatty acid ethyl esters,
the bulk production of short fatty acids (C₆ to C₁₂) esters with other metabolites,
the specific production of C₆ fatty acid esters with other metabolites,
the specific production of C₈ fatty acid esters with other metabolites,
the bulk production of enzyme bound short fatty acids (C₆ to C₁₂),
the specific production of enzyme bound C₆ fatty acids, or
the specific production of enzyme bound C₈ fatty acids.

As discussed above, the present invention further provides the use of
a protein or polypeptide according to the present invention,
a nucleic acid molecule according to the present invention, or
a host cell according to the present invention, for
the production of biofuels, such as short alkanes, short alkenes, short alkynes, short esters or alcohols,
the production of fine chemicals, such as natural compounds where preferably short fatty acids (C₆ to C₁₂) or their derivatives (such as CoA esters, methyl/ethyl esters, esters with other metabolites, alcohols) are used as building block(s), or
the production of flavoring compounds or substances, such as esters from short fatty acids (C₆ to C₁₂).

### FURTHER DESCRIPTION OF PREFERRED EMBODIMENTS

The study shown here, is the first one reported whereby rational design S. cerevisiae strains were created to produce short fatty acids. It was not only possible to show significant increase in their overall production with the highest yield of 118 mg/L (27-fold increase over the wild type) in regular YPD but also the production of specific chain lengths was demonstrated: One strain optimized for hexanoic acid (C₆) showed yields of 20 mg/L corresponding to 90% of the detected short FA (C₆ to C₁₂) in that strain. For specific octanoic acid (C₈) production, a share of 89% was shown at an output level of 48 mg/L.

### Abstract

In the present invention, the production of short fatty acids was achieved by a structural based rational design, where only key residues in FAS were changed to impose a system of chain length control.

Besides elevated overall levels of short fatty acids (118 mg/L, a 27-fold increase over the wild type) also the specific production of C₆ (20 mg/L corresponding to 90% of the detected short FA, C₆ to C₁₂) and C₈ (48 mg/L, 89% of detected short FA) was possible.

### Results

For the production of short FA in S. cerevisiae, a Δfas1 Δfas2 strain was created. Two heterozygotic strains with one deletion each, were mated and then sporulated to gain the double knockout strain. The two chains of FAS were transformed into cells on two separate low copy vectors (pRS315 and pRS313 respectively) under control of their natural promoters and terminators (Chirala 1992). The plasmid FAS system was then the only source of de-novo fatty acids.

The FAS itself, only produces FA as CoA esters and not as free acids. For S. cerevisiae several proteins were reported recently (see e.g. Knight et al., 2014) which putatively hydrolyze the short CoA esters to the free FA. These putative TEs can also contribute to the distinct product distribution due to their own substrate specificity. For the actual quantification, the media was analyzed because short chain free FA produced by S. cerevisiae are exported from the cell (Leber & da Silva, 2014).

For the production of short fatty acids, the fatty acids cycle was manipulated in all enzymatic centers that are potentially involved in chain length control (see FIG. 1), the condensation domain (KS) and the transferases (MPT and AT). While the modifying domains (KR, DH and ER) can have different affinities towards intermediates of different lengths, they do not play the key role in determining the product length.

### 1. Ketoacyl Synthase (KS) Domain:

The KS domain catalyzes the condensation step in the fatty acid synthesis, meaning the actual elongation of intermediates. Mechanistically, the catalyzed reaction works in a ping-pong mechanism: In a first step, the acyl-intermediate is loaded from the ACP into the KS where it binds covalently to C291 (ping step). When the ACP returns to the KS with a bound malonyl, the latter is decarboxylated. The produced carboxylanion reacts with the acyl chain in the KS and thereby cleaves it off from the active center of the KS (pong step).

The mutations introduced into the KS domain (see FIG. 4) for chain length control are influencing the ping step of the reaction by restricting the loading of acyl chains beyond a certain length. As an elongation is less likely (or inhibited), more short FA are produced.

In the first position, a G236S mutation was introduced. This mutation has been linked to enhanced resistance to Cerulenin, a common FAS inhibitor. In S. cerevisiae strains with this mutation used for Sake production, an increased level of C₆ and its ester derivatives was reported (Aritomi et al., 2004; see also JP 2002/027989 A).

In comparison with the wild type, the G236S strain shows a significant increase of C₆ with 15.3 mg/L on average, a 9-fold increase over the wild type (see FIG. 2).

As a second position in the KS, the neighboring M237 was mutated. Comparison of the structural data of the FAS with a bound Cerulenin (Johansson et al., 2008) and the FAS without the latter (Leibundgut et al., 2007), indicated two conformations for M237: a) It was found in a position pointing directly into the KS binding channel and b) pushed aside by Cerulenin which is mimicking a bound acyl substrate. A gatekeeper function of a methionine in this position in the KS binding channel has also previously postulated to explain the bimodal product spectrum found for the HsmtKAS (Christensen et al., 2007). Most likely the reported lower binding for C₈-ACP (Zhang et al., 2005) can be linked to the rearrangement of the methionine residue as well.

Our intention was to enhance the gatekeeper effect of this crucial position and hinder the elongation of acyl intermediates beyond certain length by replacing it with a bigger, bulkier residue. A M237W mutation was chosen.

The double mutant G236S-M237W was tested and increased levels of C₆ (with 19.9 mg/L, 12-fold increase over wild type) and C₈ (32.7 mg/L, 56-fold increase over wild type) were found (FIG. 2).

Also, a third promising position in the KS was mutated based on sequence alignments with organisms known to produce C₆ at least as an intermediate, such as Aspergillus parasiticus and Aspergillus flavus (see e.g. Hitchman et al., 2001). The F235Y mutation is located on the opposite side of the G236S-M237W mutation in the binding channel of the KS (see FIG. 4) but is believed to also impede binding of acyl chains beyond C₆. Effects of the measured G236S-F237Y double mutant strain could hardly be interpreted since its growth was highly inhibited resulting in very low short FA output. Its results in combination with mutations in the transferases are reported below.

The positions Q193, N258 and D259 are also located at the surface of the KS domain, where they interfere with interaction of the ACP and KS domain. The mutated positions are to lower probability of this interaction. Consequently, an elongation of produced fatty acids is less likely, because intermediates (which are bound to the ACP domain) are not elongated directly and the chances of an early export by the promiscuous MPT domain are increased, leading to shorter fatty acids (FA).

### 2. Malonyl-/Palmitoyl-Transferase (MPT) Domain:

In the yeast FAS systems, the MPT domain is responsible for malonyl-CoA loading and also for unloading of products (typically C₁₆ and C₁₈) as CoA esters. Similar to the KS domain, transferases in FAS also work in ping-pong mechanism. A substrate is first loaded from a CoA into the transferase where it binds covalently and is then transferred onto the ACP domain or vice versa. In the MPT specifically, both compound groups, malonyl substrates and acyl products, are transferred via this mechanism and essentially compete in the ping step for one residue, S104, in the active center. The MPT's responsibility in product cleave off made it a target for engineering.

Inherently, the MPT domain is a transferase with a generally broad substrate spectrum, which includes short acyl chains as well. The approach of destabilizing malonyl binding in the MPT and thus, shifting the equilibrium of substrate loading and product unloading in favor of the latter was implemented. It was previously shown in structural studies for other, distant related transferases, that a central arginine stabilizes the carboxyl group of malonyl (see e.g. Bunkoczi et al., 2009). In the present study, a R130K mutation was introduced in the MPT domain, which was believed to have two effects on chain length control: 1) Products could be released more easily since the active site is less often occupied with malonyl. 2) Malonyl as an elongation substrate is loaded less likely into the enzyme, which would have a comparable effect to an overall decrease in malonyl concentration; a condition which has been linked to the production of shorter fatty acids (see e.g. Kawaguchi et al., 1980).

The effects of the R130K mutation in MPT domain on the product spectrum are dramatic (FIG. 2): The overall production of short FA made up from mostly C₈ was 100 mg/L, a 23-fold increase over the wild type.

### 3. Acetyl Transferase (AT) Domain:

In the S. cerevisiae FAS, the loading of acetyl onto the enzyme is catalyzed by a second transferase, the acetyl transferase (AT). Despite the differences in substrate specificity, all acyl transferases show high homologies. Since the mentioned studies (see e.g. Bunkoczi et al., 2009) also lead to an increased acetyl binding, similar engineering on the AT domain was considered, potentially leading to an increased acetyl/malonyl ratio on the enzyme, which is beneficial for the production of short FA.

In the present study, an I306A mutation was introduced. As a second desirable effect, an acquired ability to transfer short acyl chains could open up a new way for product release.

### 4. Combinations of Mutations:

The mutations in different domains were also combined in several variations (FIG. 2), not only leading to increased overall amounts, but also several yeast strains with high chain length specificity. See Table 1.

Table 1 also gives an indication which chain length is favored with the introduction of a particular mutation. For some mutations, it can be clearly seen, that if an increased short FA output was found, this was mainly due to a higher yield in C₆ as found when the G236S or the I306A mutation is added to an existing combination. The introduction of the M237W or the R130K mutation, however, rather leads to an increase in C₈, if an increase in short FA was detected at all.

A few combinations and their effects are listed here:
The highest total yield was achieved with the combination of I306A-R130K-G236S (AT MPT KS mutant), producing a total amount of 118 mg/L.
For the specific production of C₆, the double mutant I306A-G236S (AT KS mutant) showed best results with 20 mg/L of C₆ accounting for 90% of the measured short FA (C₆ to C₁₂).
A similar specificity, but for the production for C₈, was possible with the triple mutant I306A-R130K-F265Y (AT MPT KS mutant) with a yield of 48 mg/L for C₈ specifically representing 89% of its short FA output.
With the I306A-R130K-G236S-N258A construct but a slightly different vector (promoter exchanged ADH2 promotor and buffered medium, 100 mM K2HPO4/KH2PO4), the total yield was also very high with 344 mg/L.

### Discussion

In our study, we focused on one aspect of reaction control of fatty acid production, the chain length regulation, and its rational manipulation in the enzyme responsible for its production, the fatty acid synthase. Only key residues were to be modified providing a minimally invasive system for the in vivo production of short fatty acids in S. cerevisiae. The FAS was brought into a fas1 fas2 knockout strain via a vector, but under its natural promoter and only on a low copy plasmid, mimicking natural conditions. Hence, the system was not optimized for best overall FA production but instead was rather to ensure highest possible control, also providing the ability to directly map the relations between mutations and yield in short FA.

In order to alter the product spectrum, the FAS was modified in all active centers potentially regulating chain length:
1) In the KS domain located on FAS 2, six residues were altered in order to limit elongation of intermediate products (G236S, M237W, F265Y, Q193A/E, N258A/D, D259A).
2) In the MPT domain located on FAS 1, loading of malonyl used for elongation was artificially limited and product release accordingly facilitated (R130K).
3) In the AT domain located on FAS 1, binding channels were broadened to increase acetyl influx or more likely, providing a new way for short chain product release (I306A).

The engineering presented here can be understood as metabolic engineering. Fluxes were changed rather than only the ability of one individually domain to process a certain reaction. On the FAS itself, twelve reaction steps can be catalyzed (when transferase steps are included) and this is not considering that eight of them are iteratively repeated several times until a certain chain length is reached. In this complex network of reactions which are essentially influencing each other, we were able to alter affinities and consequently change the kinetics and reaction rates with the introduction of concise mutations.

An expansive influence on the FA chain length could be shown. The effects were measured with the result of different overall amounts of short FA and a highest overall yield of 118 mg/L. The increase is 27-fold higher than in the wild type, that clearly only produces short FA as a minor byproduct in the range of 4.3 mg/L. Also distinct and controllable selectivity of chain lengths was demonstrated. For specific production of C₆, the I306A-G236S mutant was favorable (20 mg/L, 90% share C₆), for C₈, the I306A-R130K-F265Y mutant respectively (48 mg/L, 89% share of C₈).

The immediate products of the FAS, the acyl CoA esters, were not quantified but instead the extracellular free FA. This implies proteins with thioesterase activity hydrolyzing acyl CoA esters intracellularly (Knight et al., 2014). These proteins and fatty acid transporters potentially contribute to distinct product spectra.

The scope of the study was to modulate chain length in a non-invasive approach. This means that the fas1fas2 knockout strain was complemented with fas1 and fas2 variants set under their native promoters and terminators.

In our experiments, the viability of the cells was influenced by the specific mutations in the FAS. The data suggest that some of the mutations significantly influenced the fatty acid spectrum, i.e. decreasing intracellular C₁₆/C₁₈ fatty acid (CoA esters) levels, so that growth was affected. Such an impact on the viability of cells by some of our mutations is supported by supplementation studies with C_{18:1}, that generally restored normal growth behavior (Table 2). This implies that the system is highly complex and short chain fatty acid production has to be adapted to host cell architecture.

Table 1: Increase/Decrease of Yield after Introduction of Mutations.

To quantify how beneficial a mutation is, the strain without the mutation (first line) is compared to the same strain with the mutation (second line). By dividing the second value by the first value, the increase a mutation adds to the yield (for C₆, C₈ and total short FA) is given as a factor (“x-fold increase”). Accordingly, values above 1 (in bold) are equivalent to a positive, higher yield; for values below 1, the yield dropped with the introduction of the mutation.

TABLE 1
Increase/Decrease of yield after introduction of mutations.

| | | | | total |
|---|---|---|---|---|
| | | C₆ | C₈ | (C₆ to C₁₂) |
| Mutation | | (mg/L) | (mg/L) | (mg/L) |
| G236S | wild type | 1.7 | 0.6 | 4.3 |
| (KS domain) | G236S | 15.3 | 3.9 | 21.6 |
| | X-fold increase | 9.1 | 6.7 | 5.0 |
| | R130K | 15.7 | 80.2 | 100.1 |
| | R130K G236S | 9.4 | 26.7 | 39.5 |
| | X-fold increase | 0.6 | 0.3 | 0.4 |
| M237W | G236S | 15.3 | 3.9 | 21.6 |
| (KS domain) | G236S M237W | 19.9 | 32.7 | 57.2 |
| | X-fold increase | 1.3 | 8.3 | 2.6 |
| | I306A G236S | 20.4 | 1.0 | 22.8 |
| | G236S M237W I306A | 37.1 | 25.6 | 65.9 |
| | X-fold increase | 1.8 | 25.2 | 2.9 |
| | I306A R130K G236S | 52.1 | 63.1 | 118.2 |
| | G236S M237W R130K | 13.0 | 77.1 | 104.1 |
| | I306A | | | |
| | X-fold increase | 0.2 | 1.2 | 0.9 |
| F265Y | I306A R130K | 10.1 | 25.1 | 37.4 |
| (KS domain) | I306A R130K F265Y | 2.2 | 47.9 | 53.9 |
| | X-fold increase | 0.2 | 1.9 | 1.4 |
| I306A | G236S | 15.3 | 3.9 | 21.6 |
| (AT domain) | G236S I306A | 20.4 | 1.0 | 22.8 |
| | X-fold increase | 1.3 | 0.3 | 1.1 |
| | G236S M237W | 19.9 | 32.7 | 57.2 |
| | G236S M237W I306A | 37.1 | 25.6 | 65.9 |
| | X-fold increase | 1.9 | 0.8 | 1.2 |
| | R130K | 15.7 | 80.2 | 100.1 |
| | R130K I306A | 10.1 | 25.1 | 37.4 |
| | X-fold increase | 0.6 | 0.3 | 0.4 |
| R130K | wild type | 1.7 | 0.6 | 4.3 |
| (MPT | R130K | 15.7 | 80.2 | 100.1 |
| domain) | X-fold increase | 9.4 | 137.3 | 23.4 |
| | G236S | 15.3 | 3.9 | 21.6 |
| | G236S R130K | 9.4 | 26.7 | 39.5 |
| | X-fold increase | 0.6 | 6.8 | 1.8 |
| | I306A G236S | 20.4 | 1.0 | 22.8 |
| | I306A G236S R130K | 52.1 | 63.1 | 118.2 |
| | X-fold increase | 2.6 | 62.2 | 5.2 |
| | G236S M237W I306A | 37.1 | 25.6 | 65.9 |
| | G236S M237W I306A | 13.0 | 77.1 | 104.1 |
| | R130K | | | |
| | X-fold increase | 0.4 | 3.0 | 1.6 |

The following examples and drawings illustrate the present invention without, however, limiting the same thereto.

### BRIEF DESCRIPTION OF THE DRAWINGS

FIG. 1. Fatty acid cycle and its manipulation.

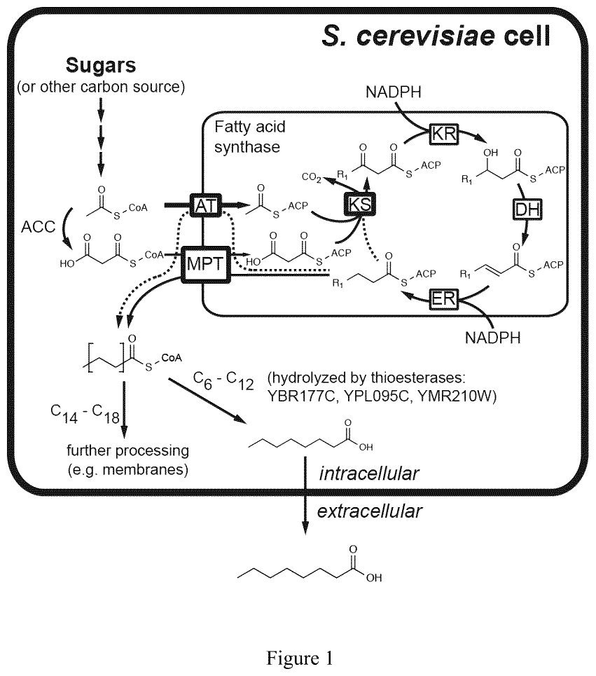

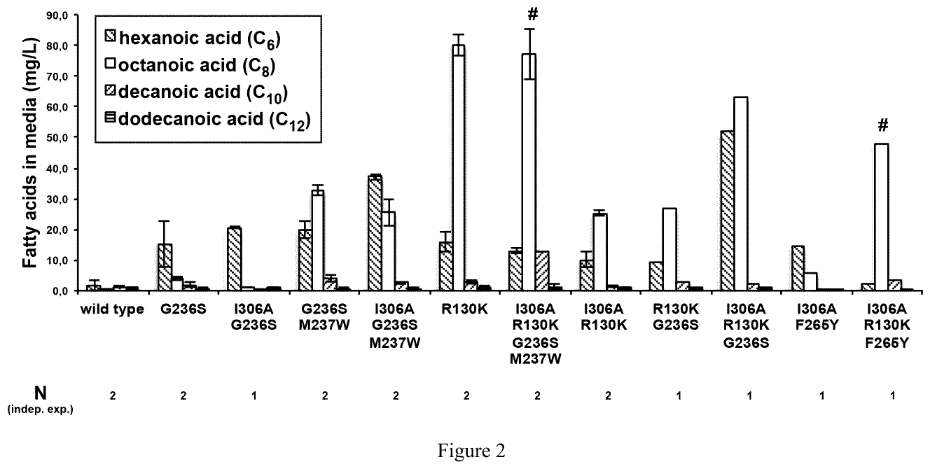

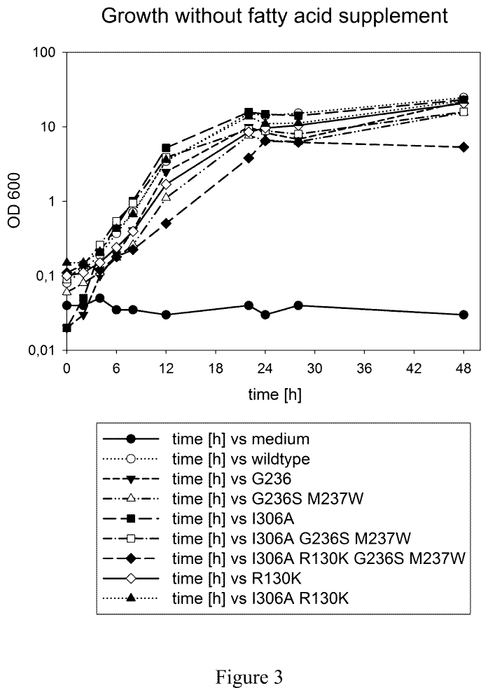

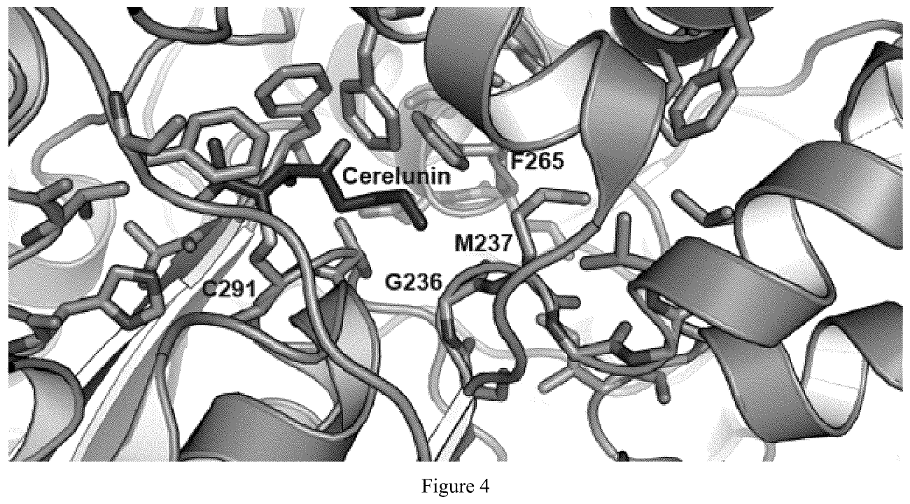

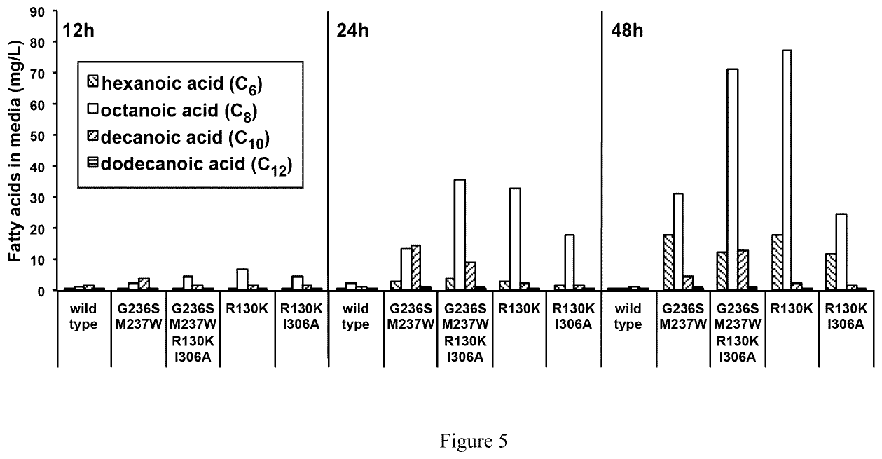

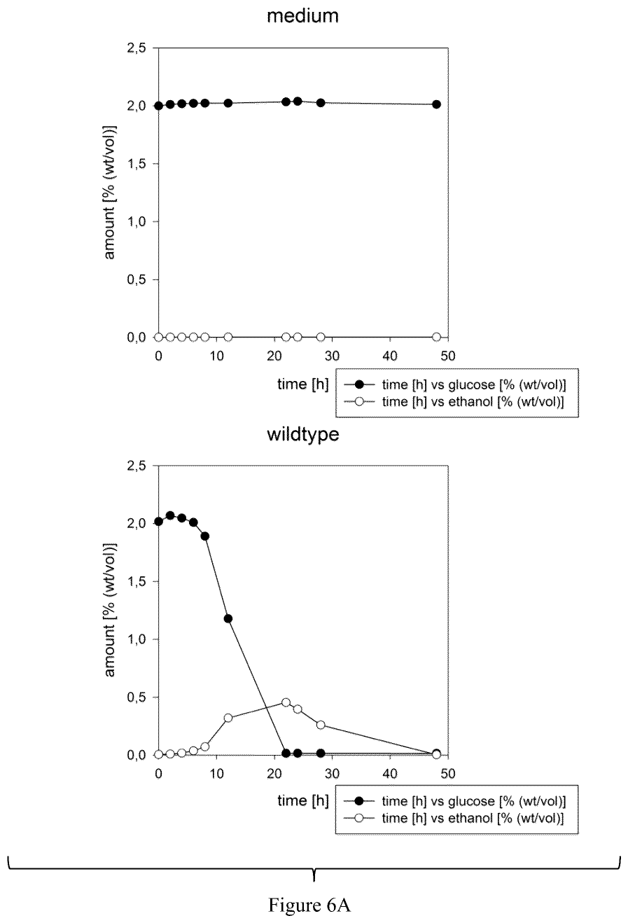

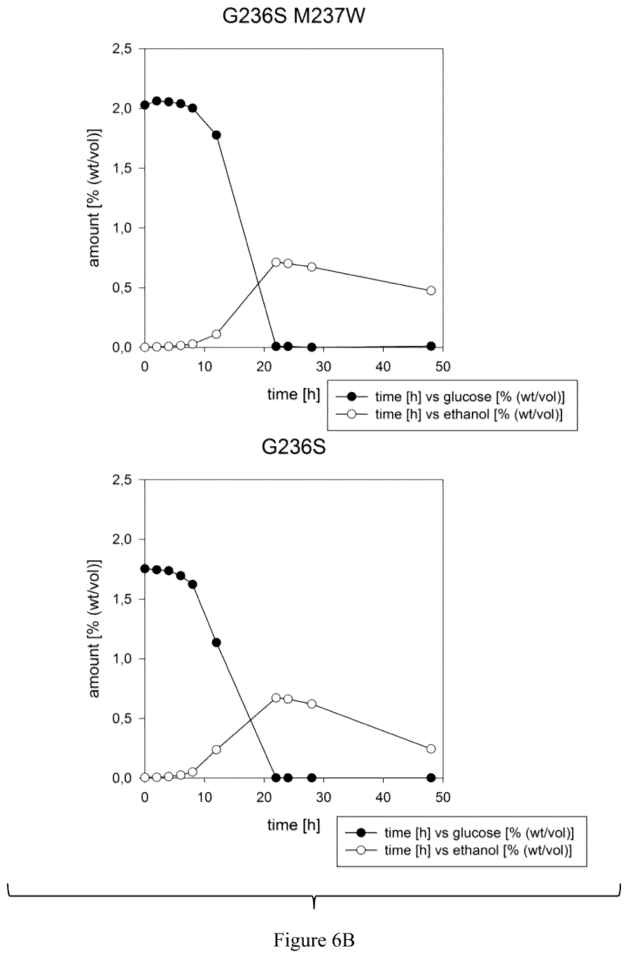

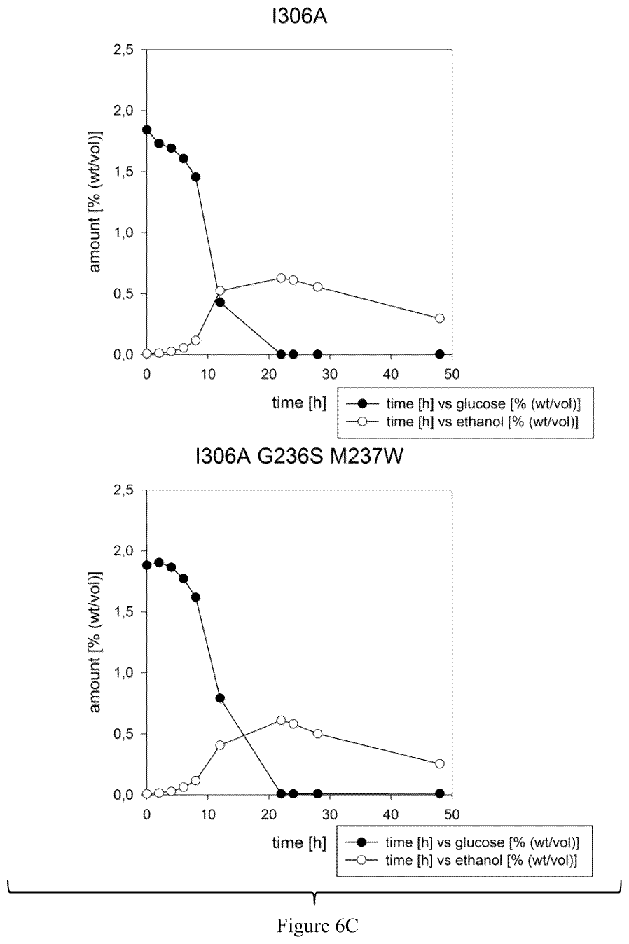

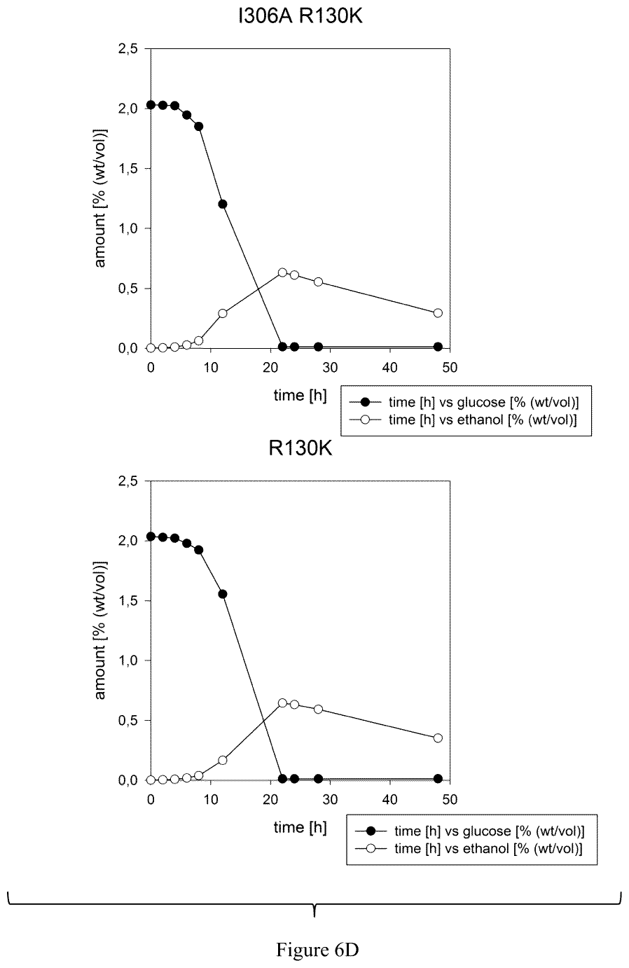

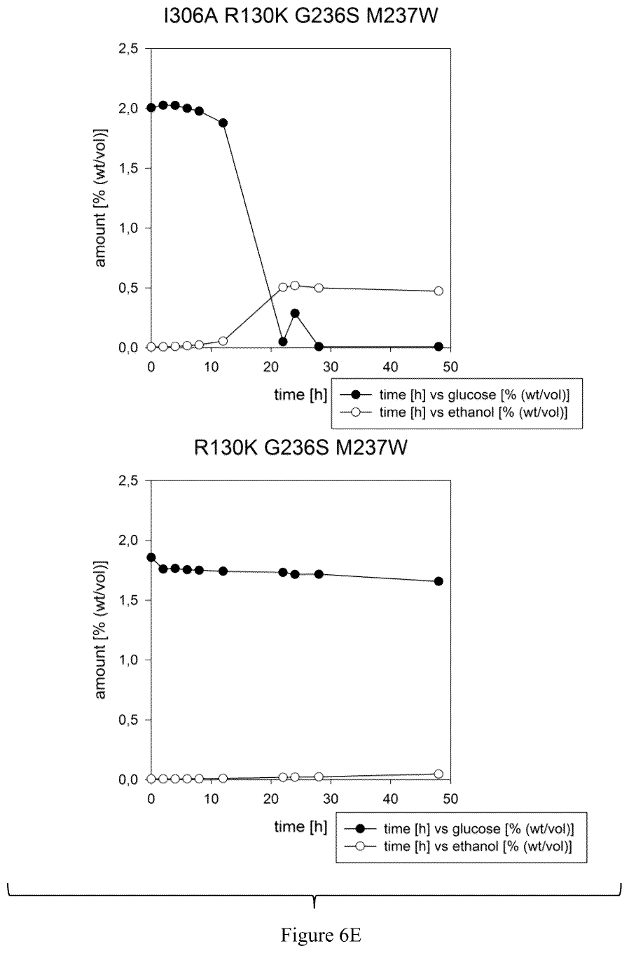

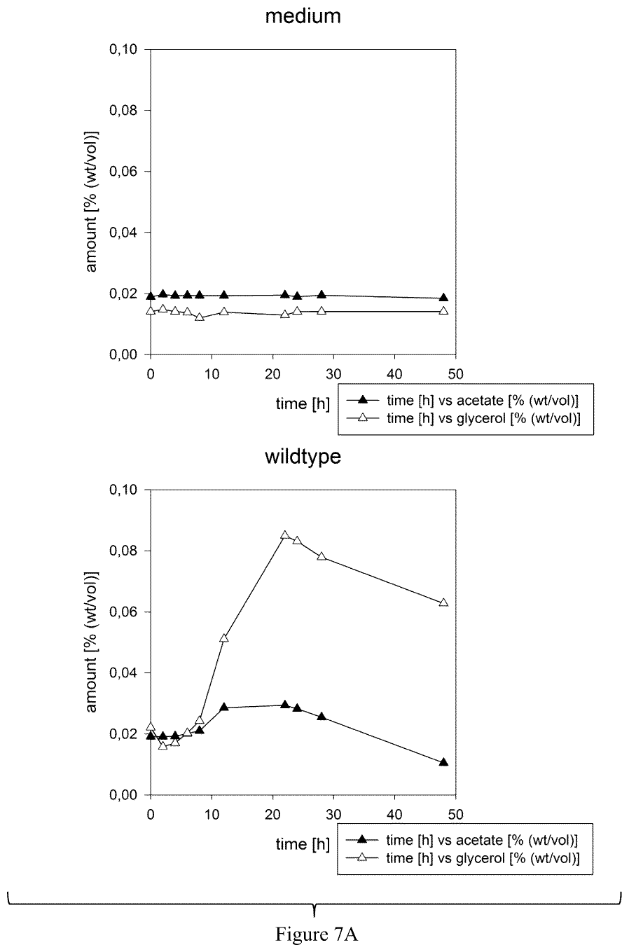

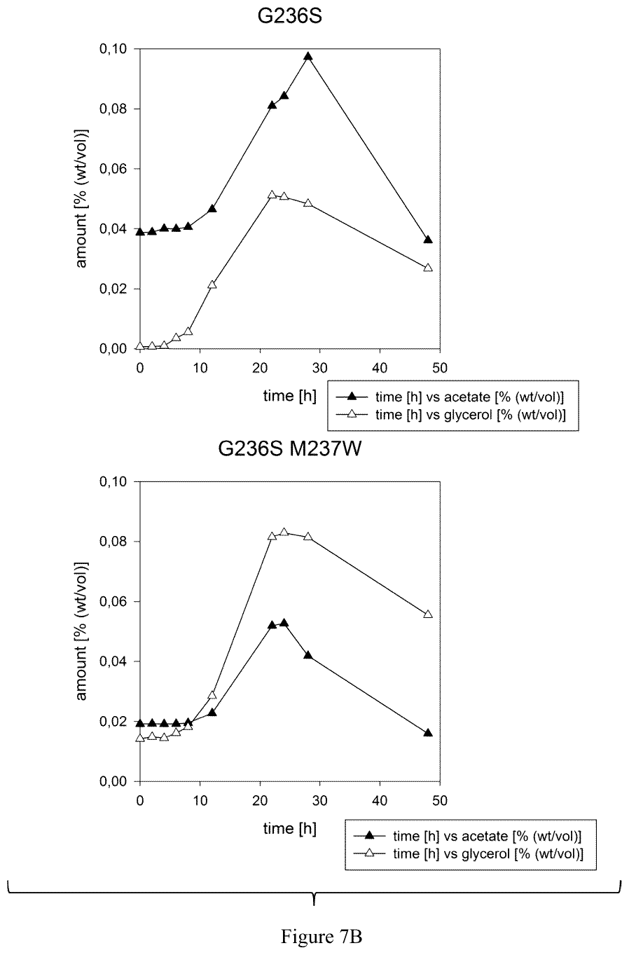

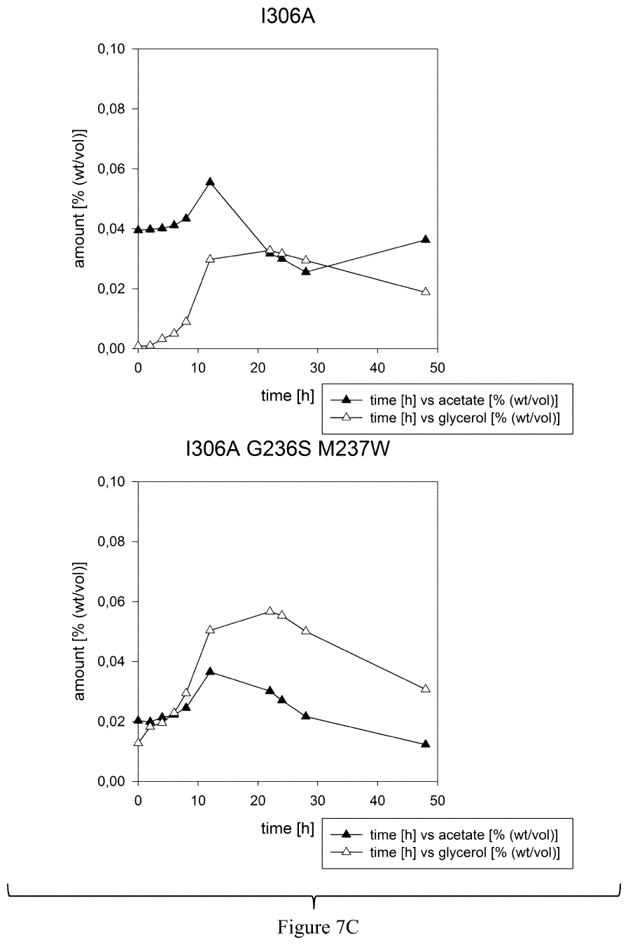

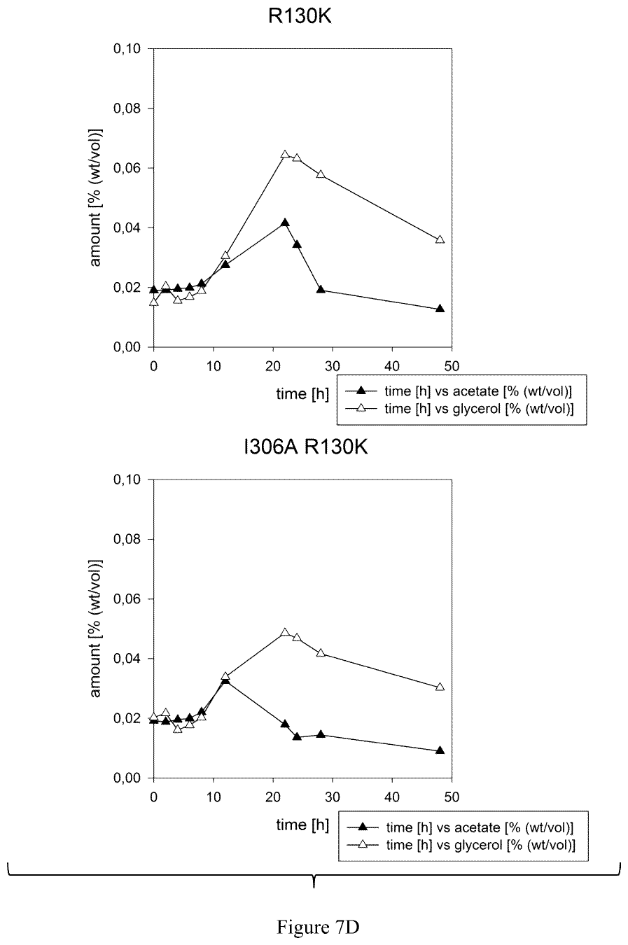

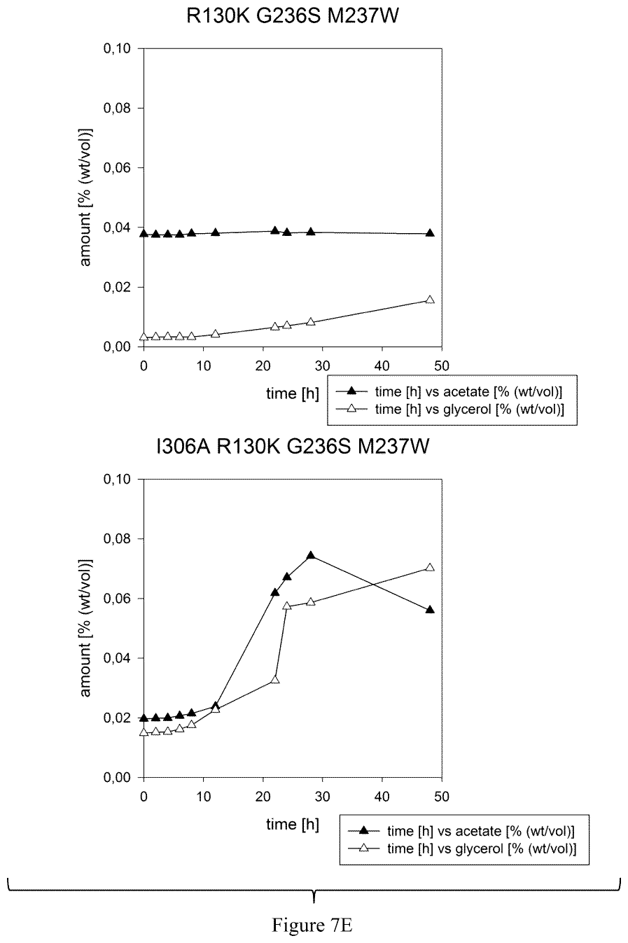

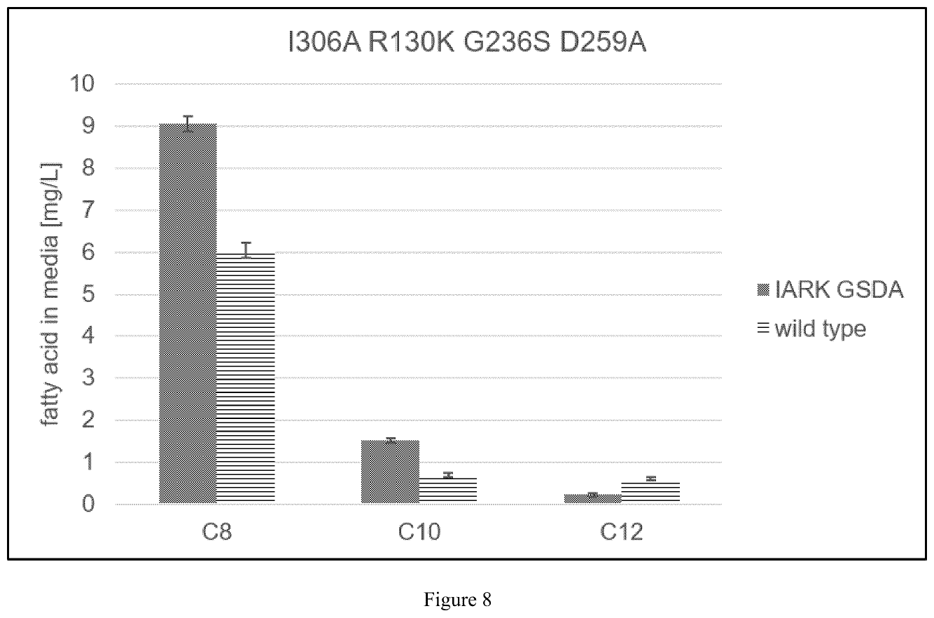

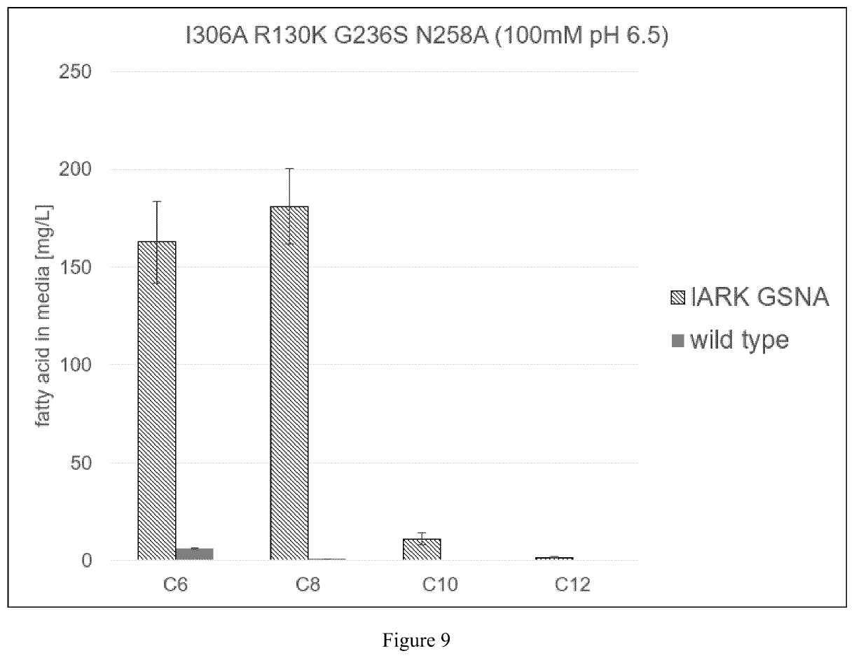

The FAS carries modifications in the KS, AT and MPT domain to produce shorter fatty acids (instead of its native product, typically C₁₆- or C₁₈-CoA) from acetyl-CoA, malonyl-CoA and NADPH. The KS mutations (G236S, M237W and F265Y) were constructed to restrict the loading of substrates beyond a certain length (indicated with a dotted line leading to the KS domain) and thus leading to the formation of shorter products. The AT (with the I306A mutation) was to enhance the loading of acetyl-CoA (indicated with the bold arrow at the AT domain) and/or act as a transferase to cleave off short chain products (dashed arrow from acyl products through the AT domain), a reaction not found in wild type FAS. The MPT mutation R130K was introduced to shift the balance in binding of malonyl and acyl chains in favor of the latter. Both the lowered malonyl loading (indicated by an arrow with a smaller tip at the MPT domain) as well as the easier acyl chain release add to an increase of short fatty acids. S. cerevisiae FAS naturally produces CoA esters, which are hydrolyzed by thioesterases, if they are shorter than a certain length. The free FA are then transported out of the cell into the media, from which they are extracted for their analysis.

FIG. 2. Product spectra of selected mutated strains in YPD.

For the measurements of the product spectra, cultures of S. cerevisiae were grown for 48 h at 30° C., the media extracted and later quantified via GC-FID. Error bars shown here reflect the standard deviation from two independent experiments (beginning from separate transformations into S. cerevisiae). The strain carrying the I306A-R130K-G236S-M11251W mutations and the strain with the I306A-R130K-F265Y mutations (both marked #) only grew to approximately one third of the regular cell density of the rest.

FIG. 3. Growing curve of manipulated Yeast strains.

For selected strains, the cell density was monitored at several time points.

FIG. 4. Binding channel of the KS domain with mutation sites.

Here, the KS domain from the S. cerevisiae FAS (light grey) is shown in cartoon depiction with important residues shown in stick representation (based on PDB code 2VKZ). The active center C291 is located on the left with a bound Cerulenin molecule (dark grey), a known FAS inhibitor mimicking a bound acyl. The binding channel extends to the right, where three mutation sites, G236S, M1250W and F265Y are shown with their initial amino acids.

FIG. 5. Product Spectra at additional time points of selected strains.

Besides the regular product spectra after 48 h, additional measurements were performed for selected strains after 12 h and 24 h.

FIGS. 6A-6E. Glucose and Ethanol concentration over time.

For selected strains, the medium was monitored at several time points during the 48 h cultivation. The amount of remaining glucose and produced ethanol in the fermentation medium was measured with HPLC.

FIGS. 7A-7E. Acetate and Glycerol concentration over time.

For the same selected strains as in FIGS. 7A-7E, also the acetate and glycerol concentrations in the medium were analyzed by HPLC.

FIG. 8. Product spectrum of a further mutated strain in YPD.

The product spectrum of a strain carrying the I306A-R130K-G236S-D259A mutations is shown in comparison to the wild type. In this case, the medium was buffered to pH 6.5 (100 mM K2HPO4/KH2PO4) and the promotor was exchanged for both the wild type and the construct to ADH2. For the measurements of the product spectrum, cultures of S. cerevisiae were grown at 30° C., cell growth was hindered and after 72 h cell density was only 5.0 (OD₆₀₀), the media extracted and later quantified via GC-FID. Error bars shown here reflect the standard deviation from three independent results (beginning from separate clones of S. cerevisiae).

FIG. 9. Product spectrum of a further mutated strain in YPD.

The product spectrum of a strain carrying the I306A-R130K-G236S-N258A mutations is shown in comparison to the wild type. In this case, the medium was buffered to pH 6.5 (100 mM K2HPO4/KH2PO4) and the promotor was exchanged for both the wild type and the construct to ADH2). For the measurements of the product spectrum, cultures of S. cerevisiae were grown at 30° C., cell growth was hindered and after 72 h cell density was only 5.0 (OD₆₀₀), the media extracted and later quantified via GC-FID. Error bars shown here reflect the standard deviation from three independent results (beginning from separate clones of S. cerevisiae).

### EXAMPLES

### Example 1

### 1. Materials and Methods

### 1.1 Description Yeast Strain

The haploid S. cerevisiae strain BY.PK1238_1A_KO, used in this work, has a BY background and the reading frames of FAS1 and FAS2 are each replaced by a kanMX4 cassette, resulting in a clean knock out of FAS I and antibiotic resistance against Geneticin. The exact genotype is Mata; ura3Δ0; his3Δ0; leu2Δ0; TRP1; lys2Δ0; MET15; fas1::uptag-kanMX4-downtag; fas2::uptag-kanMX4-downtag.

### 1.2 Vector Description

The vectors used in this work are centromeric pRS shuttle vectors of types pRS313 and pRS315 (Sikorski & Hieter, 1989) with single copy number and HIS3 and LEU2 auxotrophy marker, respectively. FAS1 or mutations thereof were always provided on pRS315, while FAS2 or mutations thereof were always provided on pRS313, each regulated by its according native promoter (995 bp upstream for FAS1 and 480 bp upstream for FAS2) (Chirala 1992). Terminator sequences were set to 295 bp and 258 bp, respectively, downstream of the open reading frames. Cloning was always done in E. coli using the Infusion HD cloning kit (Clontech, Mountain View, USA).

Wild type FAS1 and FAS2 genes were assembled from several fragments, which were amplified from S. cerevisiae genomic DNA, into pRS vectors using BamHI and SalI restriction sites. Exact chromosomal coordinates including promoter and terminator sequences according to strain S288C are for FAS1 (YKL182w): Chr XI 99676-107121 and for FAS2 (YPL231w): ChrXVI 108172-114573.

### 1.3 Primers

For the introduction of mutations by site-directed mutagenesis, the primers are listed below. The mutation site is indicated in bold typing, while the overlap between the primers is underlined.
For FAS 1 variants:

I306A forward
SEQ ID NO: 7
5′-TTCTTCGCTGGTGTTCGTTGTTACGAAGCATACCCAAACACTTCC-
3′
I306A reverse
SEQ ID NO: 8
5′-ACACCAGCG AAG AATAATACAGTAATTGCTTTTCTTACGGAGA
CG-3′
R130K forward
SEQ ID NO: 9
5′-AGTTGTGTTCTACAAAGGTATGACTATGCAAGTTGCTGTTCC-3′
R130K reverse
SEQ ID NO: 10
5′-CATAGTCATACCTTTGTAGAACACAACTTCAACTAAAGATTCGATAG
AC-3′

For FAS 2 variants:

G236S forward
SEQ ID NO: 11
5′-TCTGGTTCTTCTATGGGTGGTGTTTCTGCCTTACG-3′
G236S reverse
SEQ ID NO: 12
5′-CATAGAAGAACCAGAACAGTTACCAACCTCAGAAACATGTACG-3′
M237W forward
SEQ ID NO: 13
5′-TCTGGTTCTTCTTGGGGTGGTGTTTCTGCCTTACG-3′
M237W reverse
SEQ ID NO: 14
5′-CCAAGAAGAACCAGAACAGTTACCAACCTCAGAAACATGTACG-3′
F265Y forward
SEQ ID NO: 15
5′-ATTTTACAAGAATCA TAT ATCAACACCATGTCCGC-3′
F265Y reverse
SEQ ID NO: 16
5′-TGATTCTTGTAAAATATCATTTTGGACAGGC-3′

### 1.4 Transformation

For yeast transformation, approximately 1 μg of each plasmid DNA was co-transformed following a modified lithium acetate protocol (Schiestl & Gietz, 1989). A 5 mL overnight culture of strain BY.PK1238_1A_KO in YPD (1% yeast extract, 2% peptone, both produced by BD, Difco Laboratories, Sparks, USA; 2% dextrose, purchased from Roth, Karlsruhe, Germany) containing 200 μg/mL Geneticin disulfate, free fatty acids (myristic, palmitic and stearic acid, each 50 μg/mL) and 1% TWEEN®20 grown at 30° C. and 200 rpm was used to inoculate a main culture in the same medium.

After shaking at 30° C. and 200 rpm to OD₆₀₀=0.8, a volume of 5 mL of this culture was harvested by centrifugation (3000 ref, 5 min, 24° C.). The cells were washed by resuspending in 1 mL water and centrifuged again. After resuspension in lithium acetate solution (0.1 M), cells were incubated for 5 min at 24° C. and centrifuged (5000 ref, 15 s, 24° C.), before the transformation mix was added (240 μL PEG 1,500 solution (50%), 76 μL water, 36 μL lithium acetate solution (1.0 M), 5.0 μL single stranded DNA solution from salmon testis (10 mg/mL), 2 μL of each plasmid DNA solution). The cell suspension was mixed well and incubated for 30 min at 30° C. followed by 20 min at 42° C. After pelleting the cells by centrifugation (4000 ref, 15 s, 4° C.), they were washed with 1 mL water, pelleted again (4000 ref, 15 s, 4° C.) and resuspended in 100 μL water. For selection of the yeast transformants, the cell suspension was spread on SCD-ura; -leu agar plates containing 200 μg/mL Geneticin disulfate, free fatty acids (myristic, palmitic and stearic acid, each 50 μg/mL) and 1% TWEEN®20.

### 1.5 Cultures for Product Analysis

For the product analysis, several colonies of the S. cerevisiae strains were picked and united in one pre-culture (5 ml YPD with 200 μg/mL Geneticin disulfate, 50 mg/ml final concentration). After shaking at 200 rpm at 30° C. overnight, the OD₆₀₀ was measured. The main culture (50 ml YPD with 200 μg/mL Geneticin disulfate, 50 mg/ml final concentration) was inoculated to OD_{600=0.1} and shaken for 48 h at 200 rpm and 30° C. Before further processing, the OD₆₀₀ was measured again.

For samples with long FA supplementation, C_{18:1} and TERGITOL™ NP-40 (solution in water, 70%) were added to all cultures to a final concentration of 1 mM or 1% in the case of TERGITOL™

### 1.6 Sample Processing

For FA extraction, a protocol similar to a previously published one (Leber & da Silva, 2014) was used: First cells were spun down at 3,500 ref for 15 min. The supernatant was aliquoted in 10 ml portions and 0.2 mg of the internal standard, heptanoic acid (C₇), was added. After acidification with 1 ml HCl (1 M), 2.5 ml of a mixture of equal amounts of methanol and chloroform were added. The samples were shaken vigorously for 5 min and then centrifuged again at 3 500 ref for 10 min. The chloroform layer was transferred to a new vial and any residual water removed. The liquid was then fully evaporated in a SPEEDVAC™

For methylation a previously published protocol was used (Ichihara & Fukubayashi, 2010).

### 1.7 Determination of Free Fatty Acid by Gas Chromatography (GC)

The resulting fatty acid methyl esters dissolved in hexane (Ichihara & Fukubayashi, 2010), were measured with a Perkin Elmer CLARUS® 400 gaschromatograph (Perkin Elmer, Rodgau, Germany) equipped with an Elite FFAP capillary column (30 m×0.25 mm, film thickness: 0.25 μm; Perkin Elmer, Rodgau, Germany) and a flame ionization detector (Perkin Elmer, Rodgau, Germany). 1 μL of the sample was analyzed after split injection (10 mL/min) and helium as carrier gas. The temperatures of the injector and detector were 200° C. and 250° C., respectively. The following temperature program was applied: 50° C. for 5 min, increase of 10° C./min to 120° C. (hold for 5 min), increase of 15° C./min to 180° C. (hold for 10 min), and increase of 20° C./min to 220° C. for 37 min.

### 1.8 Metabolite Analysis by HPLC

For quantification of glucose, ethanol, glycerol and acetate 450 μL cell-free samples were mixed with 50 μL of 50% (w/v) 5-sulfosalicylic acid, vigorously shaken and centrifuged (4° C., 5 min, 13 000 ref). The supernatant was analyzed with an UHPLC+ system (Dionex UltiMate 3000, Thermo Scientific, Dreieich, Germany) equipped with a HYPERREZ™ XP Carbohydrate H+ 8 μm column. To detect the substrates a refractive index detector (Thermo Shodex RI-101) was used. Separation was carried out at 65° C. with 5 mM sulfuric acid as mobile phase (flow rate of 0.6 ml/min). Five standards (mixtures of D-glucose, ethanol, glycerol and acetate with concentrations of 0.05-2% (w/v)) were analyzed for quantification of the different compounds.

### 1.9 Determination of Cell Density

The cell density in a liquid culture was measured with an ULTROSPEC® 2100 pro spectrophotometer (GE Healthcare, USA) by determination of the optical density at 600 nm (OD₆₀₀).

### 2. Results

For the production of short FA in S. cerevisiae, a Δfas1 Δfas2 strain was created. Two heterozygotic strains with one deletion each, were mated and then sporulated to gain the double knockout strain. The two chains of FAS were transformed into cells on two separate low copy vectors (pRS315 and pRS313 respectively) under control of their natural promoters and terminators (Chirala 1992). The plasmid FAS system was then the only source of de-novo fatty acids.

2.1 Short FA Yield in YPD Supplemented with C₁₈:1

In some strains, growth was severely inhibited in regular YPD media, most likely because the plasmid FAS system did not produce enough long fatty acids necessary for cell growth. As an alternative, strains were also tested in YPD media supplemented with oleic acid (C_{18:1}, 1 mM) where all strains showed similar growth (Table 2). The reestablishment in growth is proof that an insufficient production of long chains prevented cells from growing before.

### 2.2 Vitality Parameters

In our in vivo study, the cells themselves were closely monitored. The cell density was measured for all samples at the end of the growing period and the wet cell pellet weight was noted (Table 2). In addition, for selected samples OD₆₀₀ was recorded at several time points (FIG. 3). For the growth in regular YPD, three groups can be distinguished: Strains that showed regular growth, ones with reduced growth and a group, were only an extremely low or no detectable growth could be seen. When the media was supplemented with C_{18:1} (1 mM) all strains showed similar growth.

In order to test the theory, that reduced growth for the I306A-R130K-G236S-M237W mutant could derive from a strong initial production of C₈ eventually inhibiting further growth, product spectra of selected strains were measured after 12 h and 24 h in addition to the regular measurements after 48 h (FIG. 5). While cell density of the I306A-R130K-G236S-M237W mutant reached its stationary phase already after 24 h instead of a later time (as for the R130K mutant for example, FIG. 3), the FA production follows a similar pattern for both the samples.

TABLE 2
Cell density (OD₆₀₀ and wet pellet weight) after 48 h.

| | Samples | | Samples in | |
|---|---|---|---|---|
| | in regular | | YPD with | |
| | YPD | | C_{18:1} | |
| | | wet cell | | wet cell |
| | OD₆₀₀ | pellet (g) | OD₆₀₀ | pellet (g) |
| wild type | 24.7 | 1.1 | 17.4 | 1.3 |
| G236S | 21.9 | 1.1 | 19.2 | 1.4 |
| G236S M237W | 15.6 | 1.0 | 19.5 | 1.5 |
| I306A | 23.1 | 1.1 | 19.2 | 1.4 |
| I306A G236S M237W | 15.9 | 1.1 | 18.8 | 1.4 |
| R130K G236S M237W | 3.5 | 0.2 | 22.0 | 1.5 |
| I306A R130K G236S M237W | 5.4 | 0.5 | 20.4 | 1.7 |
| R130K | 20.7 | 1.1 | 24.1 | 1.4 |
| I306 R130K | 22.5 | 1.2 | 20.3 | 1.5 |
| I306A G236S | 15.6 | 1.3 | 18.9 | 1.4 |
| R130K G236S | 10.2 | 0.9 | 19.6 | 1.4 |
| I306A R130K G236S | 14.0 | 1.1 | 18.3 | 1.3 |
| G236S F265Y | 0.1 | 0.3 | 17.5 | 1.4 |
| I306A G236S F265Y | 0.3 | 0.3 | 16.7 | 1.3 |
| R130K G236S F265Y | 0.1 | 0.3 | 15.8 | 1.4 |
| I306A R130K G236S F265Y | 0.1 | 0.3 | 17.0 | 1.5 |
| I306A F265Y | 15.6 | 1.2 | 16.9 | 1.3 |
| I306A G236S M237W F265Y | — * | — * | 18.2 | 1.4 |
| I306A F265W | — * | — * | 17.7 | 1.3 |
| I306A G236S F265W | — * | — * | 21.8 | 1.4 |
| I306A G236S M237W F265W | — * | — * | 18.4 | 1.3 |
| I306A R130K F265Y | 4.3 | 0.6 | 21.1 | 1.4 |
| I306A R130K G236S M237W | — * | — * | 20.5 | 1.3 |
| F265Y | | | | |
| I306A R130K F265W | — * | — * | 23.6 | 1.4 |
| I306A R130K G236S F265W | — * | — * | 15.8 | 1.4 |
| I306A R130K G236S M237W | — * | — * | 21.1 | 1.3 |
| F265W | | | | |

Just before further processing, the OD₆₀₀ was measured for selected samples, both when they were grown in regular YPD and in YPD supplemented with C_{18:1} (1 mM). For the growth in regular YPD, samples could be divided into three groups: regular growth (white background), reduced growth (light gray background) and very little/no growth (dark gray background). In YPD supplemented with C_{18:1} all samples showed nearly the same densities. Also, the wet pellet weight was noted. It is, however, prone to errors since residual media that is stuck to the tube can make the results less reliable. For samples marked with an asterisk (*), no main culture was grown, after the preculture already showed no significant growth.

### 2.3 Glucose Consumption, Ethanol Production, FA Production on Ethanol

For cells grown with YPD media the glucose consumption and ethanol synthesis was measured. For all tested strains (FIGS. 6A-6E) glucose is entirely consumed after 20 h, whereas the synthesis of ethanol starts after approx. 10 h, when a low glucose concentration (<1.2% (wt/vol)) is measurable. Corresponding to this, the amount of glycerol and acetate (FIGS. 7A-7E) increases after 10 h of fermentation, which can be compared to the strain with the wild type FAS.

### Example 2

### 1. Materials and Methods

For this example, materials and methods were the same as in Example 1, if not stated otherwise.

### 1.1 Vector Description

See Example 1. For constructs carrying any of the mutations Q193A, Q193E, N258A, N258D, D259A, the ADH2 promotor was used. For comparison, also one wild type construct was cloned with this promotor and used as a reference when constructs with this particular promotor were tested.

### 1.2 Primers

For the introduction of the point mutations in a PCR, the primers are listed below. The PCR products were then cloned into the vector containing the ADH2 promotor via homologous recombination. fw=forward, rv=reverse

FAS2-D259A_rv
SEQ ID NO: 17
TCTTGTAAAATAGCATTTTGGACAGGCTCATCCTTGAAACGGTCCTTAA
AC
FAS2-D259A-fw
SEQ ID NO: 18
CCTGTCCAAAATGCTATTTTACAAGAATCATTTATCAACACCATGTCCG
CTTGGG
FAS2-N258A_rv
SEQ ID NO: 19
TCTTGTAAAATATCAGCTTGGACAGGCTCATCCTTGAAACGGTCCTTAA
AC
FAS2-N258A_fw
SEQ ID NO: 20
CCTGTCCAAGCTGATATTTTACAAGAATCATTTATCAACACCATGTCCG
CTTGGG
FAS2_Q193A_rv
SEQ ID NO: 21
GAATAATGTGATTGGGTCAACCGCAGAAATGATATCATCAGAGATACCA
TAAGTC
FAS2_Q193A_fw
SEQ ID NO: 22
CTGCGGTTGACCCAATCACATTATTCGTTTTGGTCTCTGTTGTGGAAG
FAS2_Q193E_rv
SEQ ID NO: 23
GAATAATGTGATTGGGTCAACCTCAGAAATGATATCATCAGAGATACCA
TAAGTC
FAS2_Q193E_fw
SEQ ID NO: 24
CTGAGGTTGACCCAATCACATTATTCGTTTTGGTCTCTGTTGTGGAAG
FAS2_N258D_rv
SEQ ID NO: 25
TCTTGTAAAATATCATCTTGGACAGGCTCATCCTTGAAACGGTCCTTAA
AC
FAS2_N258D_fw
SEQ ID NO: 26
CCTGTCCAAGATGATATTTTACAAGAATCATTTATCAACACCATGTCCG
CTTGGG

The following primers were designed for homologous recombination of the cut vector and the ADH2 promotor.

pRS315-pADH2
SEQ ID NO: 27
CAGTGAATTGTAATACGACTCACTATAGGGCGAATTGGAGCTGGCAAAA
CGTAGGGGCAAACAAACG
pADH2-Fas2
SEQ ID NO: 28
GCAAAATATGAGCTAATTCTTGCTCAACTTCCGGCTTCATTGTGTATTA
CGATATAGTTAATAG
Fas2-pADH2
SEQ ID NO: 29
GCATACAATCAACTATCAACTATTAACTATATCGTAATACACAATGAAG
CCGGAAGTTGAGCAAGAATTAG
pRS42_hxt7
SEQ ID NO: 30
CACACAGGAAACAGCTATGAC

### 1.3 Cultures for Product Analysis

In contrast to the procedure described in Example 1, the main culture was buffered to pH 6.5 ((100 mM K₂HPO₄/KH₂PO₄). Cell cultivation was 72 hours.

The features disclosed in the foregoing description, in the claims and/or in the accompanying drawings may, both separately and in any combination thereof, be material for realizing the invention in diverse forms thereof.

### REFERENCES

Aritomi K, et al. (2004) Self-cloning Yeast Strains Containing Novel <I>FAS2</I> Mutations Produce a Higher Amount of Ethyl Caproate in Japanese Sake TI. Bioscience, Biotechnology, and Biochemistry 68(1):206-214.
Beld J, Lee D J, & Burkart M D (2015) Fatty acid biosynthesis revisited: structure elucidation and metabolic engineering. Molecular BioSystems 11(1):38-59.
Bunkoczi G, et al. (2009) Structural Basis for Different Specificities of Acyltransferases Associated with the Human Cytosolic and Mitochondrial Fatty Acid Synthases. Chemistry & Biology 16(6):667-675.
Chirala S S (1992) Coordinated regulation and inositol-mediated and fatty acid-mediated repression of fatty acid synthase genes in Saccharomyces cerevisiae. Proceedings of the National Academy of Sciences of the United States of America 89(21):10232-10236.
Choi Y J & Lee S Y (2013) Microbial production of short-chain alkanes. Nature 502(7472):571-574.
Christensen C E, Kragelund B B, von Wettstein-Knowles P, & Henriksen A (2007) Structure of the human β-ketoacyl [ACP] synthase from the mitochondrial type II fatty acid synthase. Protein Science 16(2):261-272.
Grininger M (2014) Perspectives on the evolution, assembly and conformational dynamics of fatty acid synthase type I (FAS I) systems. Current Opinion in Structural Biology 25(0):49-56.
Hitchman T S, et al. (2001) Hexanoate Synthase, a Specialized Type I Fatty Acid Synthase in Aflatoxin B1 Biosynthesis. Bioorganic Chemistry 29(5):293-307.
Ichihara Ki & Fukubayashi Y (2010) Preparation of fatty acid methyl esters for gas-liquid chromatography. Journal of Lipid Research 51(3):635-640.
Jenni S, et al. (2007) Structure of Fungal Fatty Acid Synthase and Implications for Iterative Substrate Shuttling. Science 316(5822):254-261.
Johansson P, et al. (2008) Inhibition of the fungal fatty acid synthase type I multienzyme complex. Proceedings of the National Academy of Sciences 105(35):12803-12808.
Kawaguchi A, Arai K, Seyama Y, Yamakawa T, & Okuda S (1980) Substrate Control of Termination of Fatty Acid Biosynthesis by Fatty Acid Synthetase from Brevibacterium ammoniagenes TI. The Journal of Biochemistry 88(2):303-306.
Knight M J, Bull I D, & Curnow P (2014) The yeast enzyme Eht1 is an octanoyl-CoA:ethanol acyltransferase that also functions as a thioesterase. Yeast 31(12):463-474.
Leber C & Da Silva N A (2014) Engineering of Saccharomyces cerevisiae for the synthesis of short chain fatty acids. Biotechnology and Bioengineering 111(2):347-358.
Leber C, Polson B, Fernandez-Moya R, & Da Silva N A (2015) Overproduction and secretion of free fatty acids through disrupted neutral lipid recycle in Saccharomyces cerevisiae. Metabolic Engineering 28(0):54-62.
Leibundgut M, Jenni S, Frick C, & Ban N (2007) Structural Basis for Substrate Delivery by Acyl Carrier Protein in the Yeast Fatty Acid Synthase. Science 316(5822):288-290.
Liu H, et al. (2012) Production of extracellular fatty acid using engineered Escherichia coli. Microbial Cell Factories 11.
Peralta-Yahya, P. P., Zhang, F., del Cardayre, S. B. & Keasling, J. D. (2012) Microbial engineering for the production of advanced biofuels. Nature 488: 320-328.
Runguphan W & Keasling J D (2014) Metabolic engineering of Saccharomyces cerevisiae for production of fatty acid-derived biofuels and chemicals. Metabolic Engineering 21(0):103-113.
Schiestl R & Gietz R D (1989) High efficiency transformation of intact yeast cells using single stranded nucleic acids as a carrier. Curr Genet 16(5-6):339-346.
Sikorski R S & Hieter P (1989) A System of Shuttle Vectors and Yeast Host Strains Designed for Efficient Manipulation of DNA in Saccharomyces Cerevisiae. Genetics 122(1):19-27.
Tehlivets O, Scheuringer K, & Kohlwein S D (2007) Fatty acid synthesis and elongation in yeast. Regulation of Lipid Metabolism in Yeast 1771(3):255-270.
Zhang L, Joshi A K, Hofmann J, Schweizer E, & Smith S (2005) Cloning, Expression, and Characterization of the Human Mitochondrial β-Ketoacyl Synthase: COMPLEMENTATION OF THE YEAST CEMI KNOCK-OUT STRAIN. Journal of Biological Chemistry 280(13):12422-12429.

1. A protein involved in fatty acid synthesis,
said protein comprising one or more polypeptide chains, wherein said polypeptide chain(s) comprise
(i) one or more subunits comprising the amino acid sequences of
SEQ ID NO: 1 (malonyl/palmitoyl transferase domain, MPT domain);
SEQ ID NO: 2 (acetyl transferase domain, AT domain), and/or
SEQ ID NO: 3 (ketoacyl synthase domain, KS domain);
(ii) at least one amino acid substitution
in the MPT domain at a position corresponding to R130 of the amino acid sequence of SEQ ID NO: 1;
in the AT domain at a position corresponding to I306 of the amino acid sequence of SEQ ID NO: 2;
and/or
in the KS domain at a position corresponding to M237 of the amino acid sequence of SEQ ID NO: 3;
wherein the amino acid sequence comprising the at least one amino acid substitution has at least 80% sequence identity to the respective amino acid sequence of SEQ ID NO: 1 and/or 2, and/or at least 80% sequence identity to the respective amino acid sequence of SEQ ID NO: 3,
provided that when the protein comprises an amino acid substitution G236S in the KS domain it comprises at least one additional amino acid substitution.

2. The protein according to claim 1, comprising at least one further amino acid substitution in the KS domain selected from a position corresponding to Q193, N258 and D259 of the amino acid sequence of SEQ ID NO: 3.

3. The protein according to claim 1, wherein the protein is type I FAS of Saccharomyces cerevisiae.

4. A polypeptide domain comprising
(i) one or more subunits comprising the amino acid sequences of
SEQ ID NO: 1 (malonyl/palmitoyl transferase domain, MPT domain);
SEQ ID NO: 2 (acetyl transferase domain, AT domain), or
SEQ ID NO: 3 (ketoacyl synthase domain, KS domain);
(ii) at least one amino acid substitution
in the MPT domain at a position corresponding to R130 of the amino acid sequence of SEQ ID NO: 1;
in the AT domain at a position corresponding to I306 of the amino acid sequence of SEQ ID NO: 2;
and/or
in the KS domain at a position corresponding to M237 of the amino acid sequence of SEQ ID NO: 3;
wherein the amino acid sequence comprising the at least one amino acid substitution has at least 80% sequence identity to the respective amino acid sequence of SEQ ID NO: 1 and/or 2, and/or at least 80% sequence identity to the respective amino acid sequence of SEQ ID NO: 3,
provided that when the polypeptide domain comprises an amino acid substitution G236S in the KS domain it comprises at least one additional amino acid substitution.

5. The polypeptide domain according to claim 4, comprising at least one further amino acid substitution in the KS domain selected from a position corresponding to Q193, N258 and D259 of the amino acid sequence of SEQ ID NO: 3.

6. The protein according to claim 1, comprising amino acid substitution(s) R130K in the MPT domain (SEQ ID NO. 1), and/or I306A in the AT domain (SEQ ID NO: 2).

7. The protein according to claim 1, comprising amino acid substitution(s) in the acyl binding channel of the KS domain, wherein the amino acid substitutions comprise amino acid substitution(s) G236S, M237W and/or F265Y in the KS domain (SEQ ID NO: 3), and/or further comprising amino acid substitution(s) Q193A, Q193E, N258A, N258D and/or D259A in the KS domain (SEQ ID NO: 3).

8. The protein according to claim 1, selected from the group of
a protein comprising the amino acid substitutions I306A and G236S;
a protein comprising the amino acid substitutions I306A, R130K and F265Y;
a protein comprising the amino acid substitutions I306A, R130K and G236S;
a protein comprising the amino acid substitution R130K;
a protein comprising the amino acid substitutions I306A, R130K, G236S and M237W;
a protein comprising the amino acid substitutions I306A, G236S and M237W;
a protein comprising the amino acid substitutions G236S and M237W;
a protein comprising the amino acid substitutions I306A and R130K;
a protein comprising the amino acid substitutions R130K and G236S;
a protein comprising the amino acid substitutions I306A and F265Y;
a protein comprising the amino acid substitutions I306A, G236S and F265Y;
a protein comprising the amino acid substitutions R130K, G236S and M237W;
a protein comprising the amino acid substitutions G236S and F265Y;
a protein comprising the amino acid substitutions R130K, G236S and F265Y;
a protein comprising the amino acid substitutions I306A, R130K, G236S and F265Y;
a protein comprising the amino acid substitutions I306A, G236S, M237W and F265Y;
a protein comprising the amino acid substitution I306A;
a protein comprising the amino acid substitution M237W;
a protein comprising the amino acid substitution F265Y;
a protein comprising the amino acid substitutions I306A and M237W;
a protein comprising the amino acid substitutions R130K and M237W;
a protein comprising the amino acid substitutions R130K and F265Y;
a protein comprising the amino acid substitutions I306A, R130K and M237W;
a protein comprising the amino acid substitutions I306A, M237W and F265Y;
a protein comprising the amino acid substitutions R130K, M237W and F265Y;
a protein comprising the amino acid substitutions G236S, M237W and F265Y;
a protein comprising the amino acid substitutions M237W and F265Y;
a protein comprising the amino acid substitutions I306A, R130K, M237W and F265Y;
a protein comprising the amino acid substitutions R130K, G236S, M237W and F265Y;
a protein comprising the amino acid substitutions I306A, R130K, G236S, M237W and F265Y,
a protein comprising the amino acid substitutions I306A, R130K, G236S and D259A;
a protein comprising the amino acid substitutions I306A, R130K, G236S, M237W and D259A;
a protein comprising the amino acid substitutions I306A, R130K, G236S and N258A;
a protein comprising the amino acid substitutions I306A, R130K, G236S, M237W and N258A;
a protein comprising the amino acid substitutions I306A, R130K, G236S and N258D;
a protein comprising the amino acid substitutions I306A, R130K, G236S, M237W and N258D;
a protein comprising the amino acid substitutions I306A, R130K, G236S and Q193A;
a protein comprising the amino acid substitutions I306A, R130K, G236S, M237W and Q193A;
a protein comprising the amino acid substitutions I306A, R130K, G236S and Q193E; and
a protein comprising the amino acid substitutions I306A, R130K, G236S, M237W and Q193E;
wherein
R130K refers to R130K in the MPT domain of the amino acid sequence of SEQ ID NO: 1;
I306A refers to I306A in the AT domain of the amino acid sequence of SEQ ID NO: 2;
G236S refers to G236S in the KS domain of the amino acid sequence of SEQ ID NO: 3;
M237W refers to M237W in the KS domain of the amino acid sequence of SEQ ID NO: 3;
F265Y refers to F265Y in the KS domain of the amino acid sequence of SEQ ID NO: 3;
D259A refers to D259A in the KS domain of the amino acid sequence of SEQ ID NO: 3;
N258A refers to N258A in the KS domain of the amino acid sequence of SEQ ID NO: 3;
N258D refers to N258D in the KS domain of the amino acid sequence of SEQ ID NO: 3;
Q193A refers to Q193A in the KS domain of the amino acid sequence of SEQ ID NO: 3; and
Q193E refers to Q193E in the KS domain of the amino acid sequence of SEQ ID NO: 3.

9. The protein according to claim 1, selected from
variant I306A/G236S;
variant I306A/R130K/F265Y;
variant I306A/R130K/G236S;
variant R130K;
variant I306A/R130K/G236S/M237W;
variant I306A/G236S/M237W;
variant G236S/M237W;
variant I306A/R130K;
variant R130K/G236S;
variant I306A/F265Y;
variant I306A/G236S/F265Y;
variant R130K/G236S/M237W;
variant G236S/F265Y;
variant R130K/G236S/F265Y;
variant I306A/R130K/G236S/F265Y;
variant I306A/G236S/M237W/F265Y;
variant I306A;
variant M237W;
variant F265Y;
variant I306A/M237W;
variant R130K/M237W;
variant R130K/F265Y;
variant I306A/R130K/M237W;
variant I306A/M237W/F265Y;
variant R130K/M237W/F265Y;
variant G236S/M237W/F265Y;
variant M237W/F265Y;
variant I306A/R130K/M237W/F265Y;
variant R130K/G236S/M237W/F265Y;
variant I306A/R130K/G236S/M237W/F265Y;
variant I306A/R130K/G236S/D259A;
variant I306A/R130K/G236S/M237W/D259A;
variant I306A/R130K/G236S/N258A;
variant I306A/R130K/G236S/M237W/N258A;
variant I306A/R130K/G236S/N258D;
variant I306A/R130K/G236S/M237W/N258D;
variant I306A/R130K/G236S/Q193A;
variant I306A/R130K/G236S/M237W/Q193A;
variant I306A/R130K/G236S/Q193E; and
variant I306A/R130K/G236S/M237W/Q193E;
wherein
R130K refers to R130K in the MPT domain of the amino acid sequence of SEQ ID NO: 1;
I306A refers to I306A in the AT domain of the amino acid sequence of SEQ ID NO: 2;
G236S refers to G236S in the KS domain of the amino acid sequence of SEQ ID NO: 3;
M237W refers to M237W in the KS domain of the amino acid sequence of SEQ ID NO: 3;
F265Y refers to F265Y in the KS domain of the amino acid sequence of SEQ ID NO: 3;
D259A refers to D259A in the KS domain of the amino acid sequence of SEQ ID NO: 3;
N258A refers to N258A in the KS domain of the amino acid sequence of SEQ ID NO: 3;
N258D refers to N258D in the KS domain of the amino acid sequence of SEQ ID NO: 3;
Q193A refers to Q193A in the KS domain of the amino acid sequence of SEQ ID NO: 3; and
Q193E refers to Q193E in the KS domain of the amino acid sequence of SEQ ID NO: 3.

10. The protein according to claim 1, which causes elevated overall production of short fatty acids, CoA esters of short fatty acids, ethyl esters of short fatty acids, esters of short fatty acids with other metabolites, and/or enzyme bound short fatty acids (C₆ to C₁₂) compared to the wild type protein(s) or the protein(s) without such amino acid substitution(s).

11. The protein according to claim 1, wherein the amino acid substitution(s) selected from
I306A and G236S;
I306A and F265Y;
I306A, G236S and M237W;
I306A, R130K and G236S;
G236S and M237W;
I306A, R130K, G236S and M237W;
R130K;
I306A, G236S and F265Y;
I306A, R130K and F265Y;
I306A and R130K;
R130K and G236S;
R130K, G236S and M237W;
G236S and F265Y;
R130K, G236S and F265Y;
I306A, R130K, G236S and F265Y;
I306A, G236S, M237W and F265Y;
I306A;
M237W;
F265Y;
I306A and M237W;
R130K and M237W;
R130K and F265Y;
I306A, R130K and M237W;
I306A, M237W and F265Y;
R130K, M237W and F265Y;
G236S, M237W and F265Y;
M237W and F265Y;
I306A, R130K, M237W and F265Y;
R130K, G236S, M237W and F265Y;
I306A, R130K, G236S, M237W and F265Y;
I306A, R130K, G236S and N258A;
I306A, R130K, G236S and N258D;
I306A, R130K, G236S, M237W and N258A; and
I306A, R130K, G236S, M237W and N258D
wherein
R130K refers to R130K in the MPT domain of the amino acid sequence of SEQ ID NO: 1;
I306A refers to I306A in the AT domain of the amino acid sequence of SEQ ID NO: 2;
G236S refers to G236S in the KS domain of the amino acid sequence of SEQ ID NO: 3;
M237W refers to M237W in the KS domain of the amino acid sequence of SEQ ID NO: 3;
F265Y refers to F265Y in the KS domain of the amino acid sequence of SEQ ID NO: 3;
N258A refers to N258A in the KS domain of the amino acid sequence of SEQ ID NO: 3;
N258D refers to N258D in the KS domain of the amino acid sequence of SEQ ID NO: 3;
increase(s) the selectivity for the production of C₆ fatty acids, C₆ fatty acid CoA esters, C₆ fatty acid ethyl esters, C₆ fatty acid esters with other metabolites, and/or enzyme bound C₆ fatty acids compared to wild type protein(s) or the protein without such amino acid substitution(s).

12. The protein according to claim 1, wherein the amino acid substitution(s) selected from
I306A, R130K and F265Y;
R130K;
I306A, R130K, G236S and M237W;
R130K and G236S;
I306A and R130K;
G236S and M237W;
I306A, R130K and G236S;
I306A, G236S and M237W;
R130K, G236S and M237W;
I306A and F265Y;
M237W;
I306A, G236S and F265Y;
G236S and F265Y;
I306A and G236S;
R130K, G236S and F265Y;
I306A, R130K, G236S and F265Y;
I306A, G236S, M237W and F265Y;
I306A;
F265Y;
I306A and M237W;
R130K and M237W;
R130K and F265Y;
I306A, R130K and M237W;
I306A, M237W and F265Y;
R130K, M237W and F265Y;
G236S, M237W and F265Y;
M237W and F265Y;
I306A, R130K, M237W and F265Y;
R130K, G236S, M237W and F265Y;
I306A, R130K, G236S, M237W and F265Y;
I306A, R130K, G236S and D259A,
I306A, R130K, G236S and N258A;
I306A, R130K, G236S and N258D;
I306A, R130K, G236S and Q193A;
I306A, R130K, G236S and Q193E;
I306A, R130K, G236S, M237W and D259A;
I306A, R130K, G236S, M237W and N258A;
I306A, R130K, G236S, M237W and N258D;
I306A, R130K, G236S, M237W and Q193A; and
I306A, R130K, G236S, M237W and Q193E
wherein
R130K refers to R130K in the MPT domain of the amino acid sequence of SEQ ID NO: 1;
I306A refers to I306A in the AT domain of the amino acid sequence of SEQ ID NO: 2;
G236S refers to G236S in the KS domain of the amino acid sequence of SEQ ID NO: 3;
M237W refers to M237W in the KS domain of the amino acid sequence of SEQ ID NO: 3;
F265Y refers to F265Y in the KS domain of the amino acid sequence of SEQ ID NO: 3;
D259A refers to D259A in the KS domain of the amino acid sequence of SEQ ID NO: 3;
N258A refers to N258A in the KS domain of the amino acid sequence of SEQ ID NO: 3;
N258D refers to N258D in the KS domain of the amino acid sequence of SEQ ID NO: 3;
Q193A refers to Q193A in the KS domain of the amino acid sequence of SEQ ID NO: 3; and
Q193E refers to Q193E in the KS domain of the amino acid sequence of SEQ ID NO: 3;
increase(s) the selectivity for the production of C₈ fatty acids, C₈ fatty acid CoA esters, C₈ fatty acid ethyl esters, C₈ fatty acid esters with other metabolites, and/or enzyme bound C₈ fatty acids compared to wild type protein(s) or the protein without such amino acid substitution(s).

13. The protein according to claim 1, wherein the amino acid substitution(s) selected from
I306A, R130K, G236S and M237W;
G236S and M237W;
R130K;
I306A, G236S and F265Y;
I306A, R130K and G236S;
R130K and G236S;
I306A and R130K;
I306A, G236S and M237W;
I306A and F265Y;
M237W;
I306A, R130K and F265Y;
R130K, G236S and M237W;
G236S and F265Y;
I306A and G236S;
R130K, G236S and F265Y;
I306A, R130K, G236S and F265Y;
I306A, G236S, M237W and F265Y;
I306A;
F265Y;
I306A and M237W;
R130K and M237W;
R130K and F265Y;
I306A, R130K and M237W;
I306A, M237W and F265Y;
R130K, M237W and F265Y;
G236S, M237W and F265Y;
M237W and F265Y;
I306A, R130K, M237W and F265Y;
R130K, G236S, M237W and F265Y;
I306A, R130K, G236S, M237W and F265Y;
I306A, R130K, G236S and D259A,
I306A, R130K, G236S and N258A;
I306A, R130K, G236S and N258D;
I306A, R130K, G236S and Q193A;
I306A, R130K, G236S and Q193E;
I306A, R130K, G236S, M237W and D259A;
I306A, R130K, G236S, M237W and N258A;
I306A, R130K, G236S, M237W and N258D;
I306A, R130K, G236S, M237W and Q193A; and
I306A, R130K, G236S, M237W and Q193E
wherein
R130K refers to R130K in the MPT domain of the amino acid sequence of SEQ ID NO: 1;
I306A refers to I306A in the AT domain of the amino acid sequence of SEQ ID NO: 2;
G236S refers to G236S in the KS domain of the amino acid sequence of SEQ ID NO: 3;
M237W refers to M237W in the KS domain of the amino acid sequence of SEQ ID NO: 3;
F265Y refers to F265Y in the KS domain of the amino acid sequence of SEQ ID NO: 3;
D259A refers to D259A in the KS domain of the amino acid sequence of SEQ ID NO: 3;
N258A refers to N258A in the KS domain of the amino acid sequence of SEQ ID NO: 3;
N258D refers to N258D in the KS domain of the amino acid sequence of SEQ ID NO: 3;
Q193A refers to Q193A in the KS domain of the amino acid sequence of SEQ ID NO: 3; and
Q193E refers to Q193E in the KS domain of the amino acid sequence of SEQ ID NO: 3;
increase(s) the selectivity for the production of C₁₀ to C₁₂ fatty acids, C₁₀ to C₁₂ fatty acid CoA esters, C₁₀ to C₁₂ fatty acid ethyl esters, C₁₀ to C₁₂ fatty acid esters with other metabolites, and/or enzyme bound C₁₀ to C₁₂ fatty acids compared to wild type protein(s) or the protein without such amino acid substitution(s).

14. A nucleic acid molecule, encoding a polypeptide domain according to claim 4.

15. A host cell, containing a nucleic acid molecule according to claim 14.

16. The host cell according to claim 15,
which has an elevated overall production of short fatty acids, CoA esters of short fatty acids, ethyl esters of short fatty acids, esters of short fatty acids with other metabolites, and/or enzyme bound short fatty acids (C₆ to C₁₂) compared to a cell not containing said nucleic acid molecule,
or which has an increased yield of C₆ fatty acids, C₆ fatty acid CoA esters, C₆ fatty acid ethyl esters, C₆ fatty acid esters with other metabolites, and/or enzyme bound C₆ fatty acids compared to a cell not containing said nucleic acid molecule,
or which has an increased yield of C₈ fatty acids, C₈ fatty acid CoA esters, C₈ fatty acid ethyl esters, C₈ fatty acid esters with other metabolites, and/or enzyme bound C₈ fatty acids compared to a cell not containing said nucleic acid molecule,
or which has an increased yield of C₁₀ to C₁₂ fatty acids, C₁₀ to C₁₂ fatty acid CoA esters, C₁₀ to C₁₂ fatty acid ethyl esters, C₁₀ to C₁₂ fatty acid esters with other metabolites, and/or enzyme bound C₁₀ to C₁₂ fatty acids compared to a cell not containing said nucleic acid molecule.

17. A method for the production of short fatty acids, CoA esters of short fatty acids, short fatty acid ethyl esters, short fatty acid esters with other metabolites, and/or enzyme bound short fatty acids (C₆ to C₁₂), comprising the expression of a nucleic acid molecule according to claim 14.

18. A method for the production of a biofuel, a flavoring compound and/or a fine chemical, the method comprising the expression of a nucleic acid molecule according to claim 14.

19. A method for:
the bulk production of short fatty acids (C₆ to C₁₂),
the specific production of C₆ fatty acids,
the specific production of C₈ fatty acids,
the bulk production of CoA esters of short fatty acids (C₆ to C₁₂),
the specific production of C₆ fatty acid CoA esters,
the specific production of C₈ fatty acid CoA esters,
the bulk production of ethyl esters of short fatty acids (C₆ to C₁₂),
the specific production of C₆ fatty acid ethyl esters,
the specific production of C₈ fatty acid ethyl esters, or
the bulk production of short fatty acids (C₆ to C₁₂) esters with other metabolites,
the specific production of C₆ fatty acid esters with other metabolites,
the specific production of C₈ fatty acid esters with other metabolites,
the bulk production of enzyme bound short fatty acids (C₆ to C₁₂),
the specific production of enzyme bound C₆ fatty acids,
the specific production of enzyme bound C₈ fatty acids,
the production of biofuels,
the production of fine chemicals, where short fatty acids (C₆ to C₁₂) or their derivatives are used as a building block, or
the production of flavoring substances, wherein said method comprises the use of a nucleic acid molecule according to claim 14.

20. The host cell according to claim 15, wherein said host cell is a Saccharomyces cerevisiae.


SEQUENCE LISTING (30 sequences)

SEQ ID NO: 1 - protein, 429 aa, Saccharomyces cerevisiae
SILDIVINNPVNLTIHFGGEKGKRIRENYSAMIFETIVDGKLKTEKIFKEINEHSTSYTFRSEKGLLSATQFTQPALTLMEKAAFEDLKSKGLIPADATFAGHSLGEYAALASLADVMSIESLVEVVFYRGMTMQVAVPRDELGRSNYGMIAINPGRVAASFSQEALQYVVERVGKRTGWLVEIVNYNVENQQYVAAGDLRALDTVTNVLNFIKLQKIDIIELQKSLSLEEVEGHLFEIIDEASKKSAVKPRPLKLERGFACIPLVGISVPFHSTYLMNGVKPFKSFLKKNIIKENVKVARLAGKYIPNLTAKPFQVTKEYFQDVYDLTGSEPIKEIIDNWEKYEQSMKPEVEQELAHILLTELLAYQFASPVRWIETQDVFLKDFNTERVVEIGPSPTLAGMAQRTLKNKYESYDAALSLHREILCYS

SEQ ID NO: 2 - protein, 551 aa, Saccharomyces cerevisiae
MDAYSTRPLTLSHGSLEHVLLVPTASFFIASQLQEQFNKILPEPTEGFAADDEPTTPAELVGKFLGYVSSLVEPSKVGQFDQVLNLCLTEFENCYLEGNDIHALAAKLLQENDTTLVKTKELIKNYITARIMAKRPFDKKSNSALFRAVGEGNAQLVAIFGGQGNTDDYFEELRDLYQTYHVLVGDLIKFSAETLSELIRTTLDAEKVFTQGLNILEWLENPSNTPDKDYLLSIPISCPLIGVIQLAHYVVTAKLLGFTPGELRSYLKGATGHSQGLVTAVAIAETDSWESFFVSVRKAITVLFFIGVRCYEAYPNTSLPPSILEDSLENNEGVPSPMLSISNLTQEQVQDYVNKTNSHLPAGKQVEISLVNGAKNLVVSGPPQSLYGLNLTLRKAKAPSGLDQSRIPFSERKLKFSNRFLPVASPFHSHLLVPASDLINKDLVKNNVSFNAKDIQIPVYDTFDGSDLRVLSGSISERIVDCIIRLPVKWETTTQFKATHILDFGPGGASGLGVLTHRNKDGTGVRVIVAGTLDINPDDDYGFKQEIFDVT

SEQ ID NO: 3 - protein, 650 aa, Saccharomyces cerevisiae
LERVIVVTGFAEVGPWGSARTRWEMEAFGEFSLEGCVEMAWIMGFISYHNGNLKGRPYTGWVDSKTKEPVDDKDVKAKYETSILEHSGIRLIEPELFNGYNPEKKEMIQEVIVEEDLEPFEASKETAEQFKHQHGDKVDIFEIPETGEYSVKLLKGATLYIPKALRFDRLVAGQIPTGWNAKTYGISDDIISQVDPITLFVLVSVVEAFIASGITDPYEMYKYVHVSEVGNCSGSGMGGVSALRGMFKDRFKDEPVQNDILQESFINTMSAWVNMLLISSSGPIKTPVGACATSVESVDIGVETILSGKARICIVGGYDDFQEEGSFEFGNMKATSNTLEEFEHGRTPAEMSRPATTTRNGFMEAQGAGIQIIMQADLALKMGVPIYGIVAMAATATDKIGRSVPAPGKGILTTAREHHSSVKYASPNLNMKYRKRQLVTREAQIKDWVENELEALKLEAEEIPSEDQNEFLLERTREIHNEAESQLRAAQQQWGNDFYKRDPRIAPLRGALATYGLTIDDLGVASFHGTSTKANDKNESATINEMMKHLGRSEGNPVIGVFQKFLTGHPKGAAGAWMMNGALQILNSGIIPGNRNADNVDKILEQFEYVLYPSKTLKTDGVRAVSITSFGFGQKGGQAIVVHPDYLYGA

SEQ ID NO: 4 - DNA, 1287 bp, Saccharomyces cerevisiae
  source 1..1287: mol_type = genomic DNA
tctatcttagacattgtcattaacaacccagttaacttaacaattcacttcggtggtgaaaagggtaagaggatcagagaaaactattctgctatgatctttgagactatcgtggatggaaaattgaagactgaaaaaattttcaaggaaattaatgagcacagtacttcttacacatttagatctgaaaaaggtttattgtctgctactcaatttacacaaccagctttaactttgatggaaaaagctgctttcgaagacttgaaatctaaaggtttgatcccagccgatgctacttttgctggtcactctttaggtgagtatgctgctttggcctctttggctgatgttatgtctatcgaatctttagttgaagttgtgttctacagaggtatgactatgcaagttgctgttccaagagatgagttgggcagatccaactatggtatgattgccattaacccaggtagagtcgctgcatcattctctcaagaagctttgcaatatgttgttgagagagttggtaagagaaccggctggttggttgaaatcgtcaactacaacgttgaaaaccaacaatatgttgcagctggtgatctaagagctttagacaccgttaccaatgttctaaacttcatcaaattacaaaaaattgatattattgaactacaaaagtccttatctttggaagaagttgaaggtcatttgtttgagatcattgacgaagcttccaagaaatctgctgtcaagcctcgcccacttaaattggagagaggttttgcttgtatcccattagttggtatttctgttcctttccattccacctacttgatgaatggtgttaaaccattcaagagtttcttgaagaagaatatcataaaagaaaatgtgaaggttgctagattggccggaaagtacattccaaacttgactgcaaaaccattccaggttactaaggaatatttccaggacgtttatgatttgactggctccgaacctatcaaggaaatcatcgacaactgggaaaagtatgaacaatccatgaagccggaagttgagcaagaattagctcatattttgctaactgaattgttagcttatcaatttgcctctcctgtgagatggattgaaactcaagatgtttttttgaaggattttaacactgaaagggttgttgaaatcggtccttctccaactttggctgggatggctcaaagaaccttgaagaataaatacgaatcttacgatgctgctctgtctttacatagagaaatcttatgctattcg

SEQ ID NO: 5 - protein, 1653 aa, Saccharomyces cerevisiae
ATGGACGCTTACTCCACAAGACCATTAACCCTATCTCACGGTTCTTTAGAGCACGTGCTTCTGGTACCAACCGCTTCATTTTTCATTGCTTCGCAATTACAAGAACAATTTAATAAAATTTTGCCCGAACCCACTGAAGGGTTTGCTGCAGATGACGAGCCTACCACACCTGCTGAACTAGTGGGGAAATTCCTTGGCTACGTATCTTCTCTAGTCGAACCTTCCAAGGTCGGTCAATTCGATCAGGTCTTGAACCTTTGCTTAACAGAATTTGAAAACTGTTATTTAGAAGGCAATGACATTCACGCCTTGGCTGCTAAACTATTACAGGAAAACGACACAACTTTAGTGAAGACTAAAGAACTAATTAAAAATTATATTACCGCCAGAATAATGGCTAAGAGACCATTTGACAAAAAATCCAACTCTGCTCTTTTTAGGGCCGTCGGCGAGGGTAACGCACAATTGGTAGCCATTTTCGGTGGTCAAGGTAACACCGACGACTACTTTGAAGAATTGCGTGATCTATATCAAACTTATCATGTCTTAGTGGGAGATTTAATCAAGTTCTCCGCTGAAACTTTAAGTGAACTGATTAGAACTACTTTAGATGCTGAAAAAGTCTTTACTCAAGGTTTAAACATATTGGAATGGTTGGAGAACCCTTCAAATACCCCAGACAAGGACTATTTACTTTCCATTCCAATTTCATGCCCCTTAATTGGTGTCATTCAATTGGCTCACTACGTAGTTACTGCCAAGCTTTTGGGTTTCACTCCAGGTGAGTTAAGATCTTACTTAAAAGGTGCTACAGGTCACTCTCAAGGTTTGGTTACTGCTGTCGCCATAGCTGAGACGGATTCCTGGGAATCCTTCTTCGTCTCCGTAAGAAAAGCAATTACTGTATTATTCTTCATCGGTGTTCGTTGTTACGAAGCATACCCAAACACTTCCCTACCACCATCCATCTTGGAAGATTCCTTGGAAAACAATGAAGGTGTTCCATCTCCAATGTTGTCCATTTCCAATCTAACTCAAGAACAAGTTCAAGACTATGTAAATAAGACTAACTCTCATTTGCCAGCTGGTAAACAAGTTGAAATTTCTCTAGTCAATGGTGCGAAGAATCTAGTCGTATCGGGCCCACCACAATCATTATATGGTTTAAACTTGACTTTAAGAAAGGCCAAGGCCCCATCTGGACTGGATCAATCAAGAATCCCATTCAGCGAAAGAAAATTGAAGTTCTCCAATAGGTTCTTACCTGTTGCATCACCATTCCATTCCCATCTATTGGTTCCAGCTTCAGATTTGATTAACAAAGACTTAGTCAAAAACAATGTCAGCTTTAACGCTAAAGATATTCAAATCCCCGTTTACGACACTTTTGATGGTTCAGATCTAAGAGTCCTTTCAGGTTCCATTTCCGAGAGAATCGTCGACTGCATCATTAGATTACCTGTCAAATGGGAAACTACTACACAATTCAAAGCCACCCACATATTAGACTTTGGTCCAGGTGGAGCTTCCGGTTTAGGTGTTTTAACCCATCGTAATAAAGATGGTACTGGTGTTCGTGTTATCGTTGCCGGTACTCTCGACATTAACCCAGATGATGATTACGGATTCAAGCAAGAAATCTTTGATGTTACT

SEQ ID NO: 6 - DNA, 1950 bp, Saccharomyces cerevisiae
  source 1..1950: mol_type = genomic DNA
ttggaaagagttattgtagttaccggttttgctgaagtcggcccatggggttcggccagaacaagatgggaaatggaagcttttggtgaattttcgttggaaggttgcgttgaaatggcctggattatgggcttcatttcataccataacggtaatttgaagggtcgtccatacactggttgggttgattccaaaacaaaagaaccagttgatgacaaggacgttaaggccaagtatgaaacatcaatcctagaacacagtggtatcagattgatcgaaccagagttattcaatggttacaacccagaaaagaaggaaatgattcaagaagtcattgtcgaagaagacttggaaccatttgaggcttcgaaggaaactgccgaacaatttaaacaccaacatggtgacaaagtggatatcttcgaaatcccagaaacaggagagtactctgttaagttactaaagggtgccactttatacattccaaaggctttgagatttgaccgtttggttgcaggtcaaattccaactggttggaatgctaagacttatggtatctctgatgatatcatttctcaggttgacccaatcacattattcgttttggtctctgttgtggaagcatttattgcatctggtatcaccgacccatacgaaatgtacaaatacgtacatgtttctgaggttggtaactgttctggttctggtatgggtggtgtttctgccttacgtggtatgtttaaggaccgtttcaaggatgagcctgtccaaaatgatattttacaagaatcatttatcaacaccatgtccgcttgggttaatatgttgttgatttcctcatctggtccaatcaagacacctgttggtgcctgtgccacatccgtggaatctgttgacattggtgtagaaaccatcttgtctggtaaggctagaatctgtattgtcggtggttacgatgatttccaagaagaaggctcctttgagttcggtaacatgaaggccacttccaacactttggaagaatttgaacatggtcgtaccccagcggaaatgtccagacctgccaccactacccgtaacggttttatggaagctcaaggtgctggtattcaaatcatcatgcaagctgatttagctttgaagatgggtgtgccaatttacggtattgttgccatggctgctaccgccaccgataagattggtagatctgtgccagctccaggtaagggtattttaaccactgctcgtgaacaccactccagtgttaagtatgcttcaccaaacttgaacatgaagtacagaaagcgccaattggttactcgtgaagctcagattaaagattgggtagaaaacgaattggaagctttgaagttggaggccgaagaaattccaagcgaagaccaaaacgagttcttacttgaacgtaccagagaaatccacaacgaagctgaaagtcaattgagagctgcacaacaacaatggggtaacgacttctacaagagggacccacgtattgctccattgagaggagcactggctacttacggtttaactattgatgacttgggtgtcgcttcattccacggtacatccacaaaggctaatgacaagaacgaatctgccacaattaatgaaatgatgaagcatttgggtagatctgaaggtaatcccgtcattggtgttttccaaaagttcttgactggtcatccaaagggtgctgctggtgcatggatgatgaatggtgctttgcaaattctaaacagtggtattattccaggtaaccgtaacgctgataacgtggataagatcttggagcaatttgaatacgtcttgtacccatccaagactttaaagaccgacggtgtcagagccgtgtccatcacttctttcggttttggtcaaaagggtggtcaagctattgtggttcatccagactacttatacggtgct

SEQ ID NO: 7 - DNA, 45 bp, synthetic construct
  misc_feature 1..45: note = Primer
  source 1..45: mol_type = other DNA
ttcttcgctggtgttcgttgttacgaagcatacccaaacacttcc

SEQ ID NO: 8 - DNA, 45 bp, synthetic construct
  misc_feature 1..45: note = Primer
  source 1..45: mol_type = other DNA
acaccagcgaagaataatacagtaattgcttttcttacggagacg

SEQ ID NO: 9 - DNA, 42 bp, synthetic construct
  misc_feature 1..42: note = Primer
  source 1..42: mol_type = other DNA
agttgtgttctacaaaggtatgactatgcaagttgctgttcc

SEQ ID NO: 10 - DNA, 49 bp, synthetic construct
  misc_feature 1..49: note = Primer
  source 1..49: mol_type = other DNA
catagtcatacctttgtagaacacaacttcaactaaagattcgatagac

SEQ ID NO: 11 - DNA, 35 bp, synthetic construct
  misc_feature 1..35: note = Primer
  source 1..35: mol_type = other DNA
tctggttcttctatgggtggtgtttctgccttacg

SEQ ID NO: 12 - DNA, 43 bp, synthetic construct
  misc_feature 1..43: note = Primer
  source 1..43: mol_type = other DNA
catagaagaaccagaacagttaccaacctcagaaacatgtacg

SEQ ID NO: 13 - DNA, 35 bp, synthetic construct
  misc_feature 1..35: note = Primer
  source 1..35: mol_type = other DNA
tctggttcttcttggggtggtgtttctgccttacg

SEQ ID NO: 14 - DNA, 43 bp, synthetic construct
  misc_feature 1..43: note = Primer
  source 1..43: mol_type = other DNA
ccaagaagaaccagaacagttaccaacctcagaaacatgtacg

SEQ ID NO: 15 - DNA, 35 bp, synthetic construct
  misc_feature 1..35: note = Primer
  source 1..35: mol_type = other DNA
attttacaagaatcatatatcaacaccatgtccgc

SEQ ID NO: 16 - DNA, 31 bp, synthetic construct
  misc_feature 1..31: note = Primer
  source 1..31: mol_type = other DNA
tgattcttgtaaaatatcattttggacaggc

SEQ ID NO: 17 - DNA, 51 bp, synthetic construct
  misc_feature 1..51: note = Primer
  source 1..51: mol_type = other DNA
tcttgtaaaatagcattttggacaggctcatccttgaaacggtccttaaac

SEQ ID NO: 18 - DNA, 55 bp, synthetic construct
  misc_feature 1..55: note = Primer
  source 1..55: mol_type = other DNA
cctgtccaaaatgctattttacaagaatcatttatcaacaccatgtccgcttggg

SEQ ID NO: 19 - DNA, 51 bp, synthetic construct
  misc_feature 1..51: note = Primer
  source 1..51: mol_type = other DNA
tcttgtaaaatatcagcttggacaggctcatccttgaaacggtccttaaac

SEQ ID NO: 20 - DNA, 55 bp, synthetic construct
  misc_feature 1..55: note = Primer
  source 1..55: mol_type = other DNA
cctgtccaagctgatattttacaagaatcatttatcaacaccatgtccgcttggg

SEQ ID NO: 21 - DNA, 55 bp, synthetic construct
  misc_feature 1..55: note = Primer
  source 1..55: mol_type = other DNA
gaataatgtgattgggtcaaccgcagaaatgatatcatcagagataccataagtc

SEQ ID NO: 22 - DNA, 48 bp, synthetic construct
  misc_feature 1..48: note = Primer
  source 1..48: mol_type = other DNA
ctgcggttgacccaatcacattattcgttttggtctctgttgtggaag

SEQ ID NO: 23 - DNA, 55 bp, synthetic construct
  misc_feature 1..55: note = Primer
  source 1..55: mol_type = other DNA
gaataatgtgattgggtcaacctcagaaatgatatcatcagagataccataagtc

SEQ ID NO: 24 - DNA, 48 bp, synthetic construct
  misc_feature 1..48: note = Primer
  source 1..48: mol_type = other DNA
ctgaggttgacccaatcacattattcgttttggtctctgttgtggaag

SEQ ID NO: 25 - DNA, 51 bp, synthetic construct
  misc_feature 1..51: note = Primer
  source 1..51: mol_type = other DNA
tcttgtaaaatatcatcttggacaggctcatccttgaaacggtccttaaac

SEQ ID NO: 26 - DNA, 55 bp, synthetic construct
  misc_feature 1..55: note = Primer
  source 1..55: mol_type = other DNA
cctgtccaagatgatattttacaagaatcatttatcaacaccatgtccgcttggg

SEQ ID NO: 27 - DNA, 67 bp, synthetic construct
  misc_feature 1..67: note = Primer
  source 1..67: mol_type = other DNA
cagtgaattgtaatacgactcactatagggcgaattggagctggcaaaacgtaggggcaaacaaacg

SEQ ID NO: 28 - DNA, 64 bp, synthetic construct
  misc_feature 1..64: note = Primer
  source 1..64: mol_type = other DNA
gcaaaatatgagctaattcttgctcaacttccggcttcattgtgtattacgatatagttaatag

SEQ ID NO: 29 - DNA, 71 bp, synthetic construct
  misc_feature 1..71: note = Primer
  source 1..71: mol_type = other DNA
gcatacaatcaactatcaactattaactatatcgtaatacacaatgaagccggaagttgagcaagaattag

SEQ ID NO: 30 - DNA, 21 bp, synthetic construct
  misc_feature 1..21: note = Primer
  source 1..21: mol_type = other DNA
cacacaggaaacagctatgac